(19) United States
(12) Patent Application Publication
(10) Pub. No.: US 2025/0035612 A1
(43) Pub. Date: Jan. 30, 2025

(54) NOVEL POTENCY ASSAY FOR ANTIBODY-BASED DRUGS AND USEFUL MEANS THEREFOR

(71) Applicant: NEURIMMUNE AG, Schlieren (CH)

(72) Inventor: Aubin MICHALON, Baden (CH)

(21) Appl. No.: 18/716,022

(22) PCT Filed: Dec. 5, 2022
(86) PCT No.: PCT/EP2022/084451

§ 371 Date: Jun. 3, 2024

(30) Foreign Application Priority Data
Dec. 3, 2021 (EP) 21212178.4

(51) Int. Cl. G01N 33/50; G01N 33/543; G01N 33/566; G01N 33/68

(52) U.S. Cl. CPC G01N 33/5023; G01N 33/543; G01N 33/566; G01N 33/6854; G01N 2333/4709

(57) ABSTRACT

Provided is a novel method for determining the potency of an antibody. Furthermore, methods and kits are provided for the production, quality control, and batch release of a pharmaceutical composition comprising an antibody-based drug. In addition, cyclic compounds based on peptides comprising an epitope from an amyloidogenic protein involved in systemic amyloidosis are described, which are useful in antibody potency and antibody binding assays in general as well as in screening and obtaining an antibody of interest.

### SEQUENCE LISTING

The instant application contains a Sequence Listing which has been submitted electronically in XML format and is hereby incorporated by reference in its entirety. Said XML copy, created on Aug. 23, 2024, is named “51705-011002_Sequence_Listing_8_23_24” and is 143,694 bytes in size.

### FIELD OF THE INVENTION

The present invention generally relates to a novel method of characterizing therapeutically useful antibodies and equivalent binding molecules for which antibody Fc-mediated activities play a critical role in the mechanism of action, which method is suitable as a potency assay, particularly useful for batch release of a pharmaceutical composition comprising the antibody or like binding molecule, specifically when conducting clinical trials, applying for marketing authorization and for quality control of the approved drug. In a further aspect, the present invention relates to cyclic compounds comprising peptides containing an epitope of a systemic amyloidogenic protein, which can be use in such potency assay.

### BACKGROUND OF THE INVENTION

Monoclonal antibody drugs have been maturing from a research target to an improved technology, from clinical research to commercialization over the past three decades. In recent years, the number of monoclonal antibody drugs approved for marketing has rapidly increased. with the landmark 100th monoclonal antibody product being approved by the United States Food and Drug Administration (FDA) in 2021. In 2019, nine of twenty top-selling drugs were monoclonal antibody drugs (Mullard, Nature Reviews Drug Discovery 20, 491-495 (2021), DOI: 10.1038/d41573-021-00079-7).

One promising application for therapeutic antibodies is the treatment of amyloidoses, which occur due to toxic amyloid aggregations. Neurodegenerative diseases including Alzheimer's, Parkinson's and Huntington's disease represent a highly prevalent class of fatal localized amyloidoses in which amyloid deposits form in the nervous system where they induce death of specific neuronal cell types. In systemic amyloidoses, such as immunoglobulin light chain, transthyretin and dialysis-related amyloidosis, several organs are affected as the amyloidogenic protein is distributed in different sites of the body as it travels from the site of synthesis. Antibodies and antibody fragments have been already proven to be effective anti-amyloid molecules. For example, aducanumab shows dose-dependent clearance of amyloid deposits in Alzheimer's patients and has recently been approved by the FDA for use in treatment of Alzheimer's disease.

The therapeutic utility of an antibody, in particular as a drug effective for the treatment of amyloidoses, depends not only on the ability of the antibody to bind the aggregate, but also on antibody Fc-mediated activities, which play a critical role in the mechanism of action. Binding of antibodies to Fc receptors on cell surfaces triggers a number of important and diverse biological responses including engulfment and destruction of antibody-coated particles (called antibody-dependent cellular phagocytosis, or ADCP), clearance of immune complexes, lysis of antibody-coated target cells by killer cells (called antibody dependent cell-mediated cytotoxicity, or ADCC), release of inflammatory mediators, placental transfer and control of immunoglobulin production.

One important mechanism of action (MoA) for antibodies targeting aggregated proteins, like amyloid beta (AB), is ADCP. Antibody binding to target proteins results in presentation of multivalent Fc domains that can bind to high and low affinity Fcy receptors on patrolling immune cells, such as macrophages, and recruit them to specific areas. The clustering of Fc receptors results in membrane deformation around the target antigen, activation of intracellular signal transduction, and changes in actin cytoskeletal dynamics that ultimately lead to target antigen engulfment by phagocytic cells.

Thus, antibodies and corresponding binding molecules are promising drugs for the prevention and treatment of protein aggregate diseases. However, when producing a pharmaceutical composition, it is not sufficient to formulate the drug substance into the drug product, it is also essential that the resulting drug product is approved by the regulatory body in the country in which the pharmaceutical composition is to be used. In the United States, the responsible regulatory body is the FDA (http://www.fda.gov/), and in Europe, it is for instance the European Agency for the Evaluation of Medicinal Products (EMEA) (http://www.emea.eu.int/).

The approval process is thoroughly regulated, and the drug developers are required to submit a substantial amount of information regarding the drug product candidate to the regulatory authorities to obtain approval. This may include information about the potency of the drug product candidate and corresponding assays to determine the potency. Such a potency assay serves to characterize the product, to monitor lot-to-lot consistency and to assure stability of the product.

The potency of antibodies for which Fc binding to Fc receptor plays a critical role for the mechanism of action are traditionally measured by use of biological assays in which the effect assessed is dependent on Fc-Fc receptor binding. Such assays may include ADCC, ADCP, or induction or inhibition of T cell activation requiring antibody cross-linking. However, the assays developed so far are often laborious, require expensive instruments, (e.g., flow cytometers), and are quite complex. For example, an ADCP assay is usually a two-step process, wherein the antibody needs to bind the target and the macrophages need to recognize and bind to the antibody bound to the target leading to phagocytosis. International application WO 2017/157961 A1 describes such method for assaying ADCP by measuring the uptake of aggregated proteins illustrated with Abeta.

Therefore, the assay to characterize the product, to monitor lot-to-lot consistency and to assure stability of the product is of clinical importance and should be relatively easy to handle as well as sufficiently sensitive to detect differences which may impact mechanism of action and function of the product.

### SUMMARY OF THE INVENTION

The present invention generally relates to a novel method of characterizing therapeutically useful antibodies and equivalent binding molecules for which antibody Fc-mediated activities play a critical role in the mechanism of action, which method is suitable as a potency assay, particularly useful for batch release of a pharmaceutical composition comprising the antibody or like binding molecule, specifically when conducting clinical trials, applying for marketing authorization and for quality control of the approved drug. The present invention further relates to a cyclic compound comprising a peptide or protein fragment which comprises an epitope of an antibody or equivalent binding molecule and to the use of such cyclic compound in a method of determining the potency of the antibody or binding molecule as well as in drug discovery and diagnostic field in general.

More particularly, the present invention relates to a novel method for determining the phagocytosis-related potency of a target antigen binding molecule comprising an Fc domain as well as to the use of this method in the production and quality control of a pharmaceutical composition comprising such molecule, wherein in a preferred embodiment, the target antigen is an amyloidogenic protein, preferably in an aggregated, misfolded, and non-physiological form. As illustrated in Examples 3 and 4 and the corresponding Figures, a stable and sensitive reporter gene assay has been developed which is suitable to determine the potency of an antibody and which has the capacity to detect potency loss related to Fc domain alterations. As further illustrated in Example 6 and the corresponding Figures, the performance of the reporter gene assay has been even improved by using a cyclic peptide as target antigen, which comprises an epitope of the amyloidogenic protein.

In accordance with the present invention, several experiments have been performed to apply the ADCP assay of WO 2017/157961 A1 illustrated with Abeta to a systemic amyloidogenic protein, transthyretin (TTR). In a first set of experiments an in vitro assay including human-derived macrophages, fluorescently labeled L55P-TTR protein, and an aggregated TTR selective antibody was developed; see Example 1. However, some variability in phagocytic activity between macrophages obtained from different blood donors was observed. Accordingly, to eliminate this source of variability, the in vitro phagocytosis assay was re-developed using the human monocytic THP1 cell line instead of fresh PBMCs; see Example 2, but again some variability was observed between replicates.

The present invention provides an improved assay that is particularly suitable for determining the potency of antibodies and Fc domain containing binding molecules which bind amyloidogenic TTR or other amyloidogenic proteins that are involved in systemic amyloidosis. Thus, in another set of experiments performed within the scope of the present invention different types of cellular assays have been evaluated with varying success. Eventually, as illustrated in Examples 3 and 4, it turned out that an assay making use of mammalian cells, especially Jurkat cells genetically engineered to express a human Fc receptor, in particular Fc receptor FcγRI (CD64) as effector cells gave very reliable results, especially for systemic amyloidogenic proteins such as TTR as target antigen, and that this set up is particularly suitable in a potency assay for target antigens that are present as aggregates.

In another set of experiments, a cyclic peptide comprising a TTR epitope (cyclic TTR peptide) has been used as target antigen instead of a TTR aggregate. Surprisingly, this assay showed a remarkable improvement of sensitivity and reliability. Without intending to be bound by theory, the extraordinary performance of the assay with a cyclic peptide could be due to a very stable conformation adopted by the cyclic peptide since it is constrained by having the two extremities connected together, and thus, mimics the stability of a protein aggregate. However, as shown in Example 6, even if such theoretical considerations are taken into account, the assay with the cyclic peptide as the target antigen is an order of magnitude more precise and sensitive than the assay with the target antigen present as protein aggregate. Again, without intending to be bound by theory this could be due to the smaller size of the peptide compared to the protein and the resulting higher epitope density which translates into both higher binding capacity and higher avidity effect. Moreover, the epitope in the cyclic peptide might be better accessible compared to the full-length protein. Nevertheless, since the cyclic TTR peptide including the linker amino acids with a total of 31 amino acids is only four times smaller than the full-length TTR protein, the size of the cyclic peptide cannot a priory account for the observed effect. A further reason could be the better control of the conformation for a synthetic peptide compared to a recombinant protein. In particular, more than 95% of the peptide is cyclized, whereas it is unknown what fraction of the misfolded-aggregated TTR protein adopts the amyloid conformation. Because mis. WT-TTR is a heterogenous mix of conformations, a significant fraction of the protein may form amorphous aggregates instead of amyloid. In summary, although with the results of the experiments described in appended Examples 5 to 8 good explanatory approaches and theories can now be developed in retrospect to envisage further cyclic peptides with suitable epitopes for the detection and identification of potent antibodies against amyloidogenic, in particular systemic amyloidogenic proteins, this was not foreseeable without knowledge of the present results and teaching of the present invention. In this context, and again without wishing to be bound to any theory, it is noteworthy that it has recently been shown by cryo-EM studies that the amyloid structures of systemic amyloidogenic proteins such as ATTR and AL amyloidosis caused by misfolding of immunoglobulin light chains (LCs) are on the one hand similar, but on the other hand substantially different from those of local amyloidogenic proteins such as tau; see FIG. 5 in Schmidt et al., Nat. Commun. 10 (2019), 5008, https://doi.org/10.1038/s41467-019-13038. Therefore, it is reasonable to assume that the present results for cyclic peptides derived from TTR can also be applied to other systemic amyloidogenic proteins.

Accordingly, in a further aspect, the present invention relates specifically to the provision of cyclic compounds comprising peptides containing an epitope of a systemic amyloidogenic protein, the epitope preferably being accessible to binding by an antibody only in the misfolded and/or aggregated form of the protein, as in the case of a neoepitope, and/or the epitope being at least not present in the physiologically active form of the protein, e.g. in the case of an epitope accessible in the monomer of the TTR protein, which is hidden in the physiologically active tetramer and is no longer accessible to antibody binding. As illustrated in Example 6, the cyclic compound of the present invention is particularly useful in the potency assay of the present invention.

The outstanding performance of the cyclic compound as target antigen is shown in the ELISA assays described in Example 5. EC₅₀ values for antibody binding to the cyclic peptide were compared to EC₅₀ values for antibody binding to the protein aggregate and to a linear peptide comprising the same epitope as the cyclic peptide. Again, the cyclic peptide performed best, in that it showed the highest binding affinity to the antibody, i.e., the lowest EC₅₀ value. As mentioned above, the higher binding affinity between the antibody and the cyclic peptide in comparison to the protein aggregate could be due to the higher epitope density, which results in better apparent binding affinity, due to the higher binding capacity and higher avidity effect, due to the better accessibility of the epitope in the cyclic peptide as compared to the full-length protein, and/or due to the better control of the conformation for a synthetic peptide as compared to a recombinant protein. Accordingly, in one embodiment, the cyclic compound and cyclic peptide of the present invention, respectively, provide for a higher binding affinity with an antibody than with the target protein it is derived from and higher than a corresponding linear peptide in an ELISA assay such as described in appended Example 5, preferably at least 2-fold, more preferably at least 3-, 4-, or 5-fold and most preferably at least 6-, 7- 8-, 9- or 10-fold higher compared to the full-length target protein and/or linear peptide.

The superiority of the cyclic peptide over the linear peptide of similar sequence might be explained, without being bound by theory, by the extreme flexibility of a linear peptide which therefore can adopt a virtually infinite number of conformations in contrast to a cyclic peptide which is constrained by having the two extremities connected together and has therefore much less flexibility and adopts more stable conformations. In different terms, a cyclic peptide has a lower entropy than the same amino acid sequence in a linear form.

Accordingly, the provision of the cyclic compound in accordance with the present invention represents an important contribution to the art given its outstanding utility as a suitable target to study the binding between a target antigen and a corresponding target antigen binding molecule, for example in assays which require a high sensitivity. Nevertheless, the present invention also relates to the linear form of the cyclic compound and cyclic peptide, respectively, for example for use a precursor for preparing the cyclic compound or as a control in the experiments.

As further described in Example 3, the effector cells employed in the assay of the present invention contain a reporter gene under the control of a response element that is responsive to activation by the Fc receptor. Since the reporter gene activity can be measured via standard photometers, no expensive and complex equipment is required.

In summary, a method has been developed for determining the potency of a molecule that binds to a target antigen and comprises an Fc domain, wherein the method comprises the following steps:
(a) contacting a target antigen with the binding molecule under conditions allowing the formation of a binding molecule-target antigen complex;
(b) contacting the binding molecule-target antigen complex with a population of effector cells that express an Fc receptor and harbor a reporter gene under the control of a response element that is responsive to activation by the Fc receptor under conditions allowing for binding of the Fc receptor to the Fc domain of the binding molecule wherein binding of the Fc domain to the Fc receptor results in intracellular signaling and mediates a quantifiable reporter gene activity; and
(c) detecting a signal induced by the reporter gene activity,
wherein at least one MoA of the Fc domain of the binding molecule is mediated through the binding of the Fc domain to an Fc receptor and the reporter gene activity is indicative for the potency of the binding molecule.

Such an assay can be applied in a method of producing a pharmaceutical composition comprising a target antigen binding molecule, wherein first after production, the potency of said binding molecule is analyzed. Based on the result, it is assessed whether the binding molecule may be used in a pharmaceutical composition or not. In particular, only binding molecules that are regarded as potent according to the assay are selected for further use and formulated as a pharmaceutical composition with a pharmaceutically acceptable carrier.

The potency assay of the present invention can also be used in a method for analyzing and selecting a batch of a pharmaceutical composition of a target antigen binding molecule, wherein a sample of the batch to be analyzed and a control sample are subjected to said potency assay and the reporter gene activity of the sample is compared to that of the control. The batch for which the sample shows greater, equal or not substantively less reporter gene activity compared to the control is finally chosen for further use. Thus, the method of the present invention can be used for verifying lot-to-lot consistency.

The present invention further relates to a kit which is preferably designed to carry out the method of the present invention, in particular to assay the potency of a binding molecule comprising an Fc domain to induce ADCP, wherein the kit comprises at least
(i) a population of effector cells genetically engineered to express an Fc receptor and harboring a gene encoding a reporter under control of a response element that is responsive to activation by the Fc receptor;
(ii) a corresponding substrate for the reporter; and optionally
(iii) the target antigen;
(iv) a microtiter plate, preferably a 96- or 384-well plate including a lid;
(v) recommendations for buffers, diluents, substrates and/or solutions as well as instructions for use, in particular instructions how to perform the assay of the present invention;
(vi) washing, blocking and assay/sample dilution buffer; and/or
(vii) a positive control target antigen binding molecule, preferably an antibody.

In one embodiment, the kit of the present invention comprises at least
(i) a population of effector cells genetically engineered to express an Fc receptor and harboring a gene encoding a reporter under control of a response element that is responsive to activation by the Fc receptor;
(ii) a corresponding substrate for the reporter; and
(iii) the target antigen; wherein the kit optionally further comprises
(iv) a microtiter plate, preferably a 96- or 384-well plate including a lid;
(v) recommendations for buffers, diluents, substrates and/or solutions as well as instructions for use, in particular instructions how to perform the assay of the present invention;
(vi) washing, blocking and assay/sample dilution buffer; and/or
(vii) a positive control target antigen binding molecule, preferably an antibody.

In a preferred embodiment, the kit of the present invention comprises, instead or in addition to the target antigen, a cyclic compound which comprises a peptide comprising an epitope from an amyloidogenic protein involved in systemic amyloidosis, and/or comprises a precursor of the cyclic compound, wherein the compound is in linear form, which could also serve a control similar as shown for the TTR peptide in the Examples.

The method, i.e., potency assay of the present invention has been illustrated with the amyloidogenic protein TTR and aggregates thereof and cyclic peptides comprising a TTR epitope, respectively, as the target antigen and an anti-TTR antibody, such as, e.g., NI-301.37F1, which is disclosed in international application WO 2015/092077 A1, and which has been described to be capable of activating the immune system for the elimination of TTR fibrils in an animal model; see international application WO 2020/094883 A1. TTR in its physiological form is a tetramer protein that develops amyloidogenic properties when it dissociates into monomers and forms transthyretin amyloidosis (ATTR), a systemic amyloidosis. Systemic amyloidosis is a protein misfolding disorder caused by extracellular deposition of amyloid leading to organ dysfunction while localized amyloidosis refers to intracellular and/or extracellular amyloid deposits that occur only in the organ or tissue of precursor protein synthesis such as intracellular Tau protein fibrils and extracellular amyloid-β fibrils and plaques in Alzheimer's disease. In principle, the method of the present invention is applicable to any target antigen, in particular any protein that in its pathogenic variant forms a ncocpitope, for example an epitope which is only exposed in the misfolded variant, a conformational epitope on aggregates, fibrils and/or oligomers, an epitope on extracellular variant of an otherwise physiological protein that is located intracellularly, or an epitope specific for exogenous pathogens such as fungi, bacteria, and viruses. In addition, the method of the present invention in principle can be performed with any kind of antigen including aggregates, fibrils, oligomers, (misfolded) monomers as well as protein fragments and peptides that contain and display the epitope(s) of the target antigen binding molecule to be tested, preferably wherein the peptide is provided in cyclic form. Similarly, the cyclic compound of the present invention can in principle comprise any peptide or protein fragment which is capable of forming a cyclic compound, in particular a peptide or protein fragment that comprises a neoepitope as mentioned above. In a particularly preferred embodiment, the (neo) epitope is hidden in the target antigen's naturally folded conformation but accessible to antibody binding following unfolding and aggregation, e.g., like the linear epitope WEPFA of antibody NI-301.37F1, located in position 41 45 of mature TTR protein.

Nevertheless, in accordance with the present Examples, the method of the present invention is particularly suited and thus preferred for determining the potency of antibodies targeted against an amyloidogenic protein, preferably against an aggregate of the misfolded and non-physiological form of the protein such as transthyretin and its amyloidogenic form, and against fragments and peptides of the protein, preferably in cyclic form, which comprise an epitope from the amyloidogenic protein, preferably from an epitope that is exposed in the misfolded and non-physiological form of the protein, such as transthyretin.

The method of the present invention is particularly useful for measuring antibody potency to activate ADCP.

In addition, the cyclic compound of the present invention, and the linear precursor thereof, are especially useful in methods for identifying and optionally obtaining an antibody which binds to an amyloidogenic protein involved in systemic amyloidosis, the method typically comprising the steps of:
(a) providing, optionally producing one or more potentially amyloidogenic protein binding antibodies or a source thereof;
(b) subjecting the one or more of potentially amyloidogenic protein binding antibodies or source thereof to a binding assay comprising the cyclic compound of the present invention; and
(c) identifying and optionally obtaining an antibody (subject antibody) that has been determined to bind to the cyclic compound.

This method can be combined with the potency assay of the present invention, and/or any other suitable method for further determining the diagnostic or preferably therapeutic utility of the subject antibody.

Hence, a further embodiment of the present invention consists in a method of producing a pharmaceutical composition comprising an antibody which binds to an amyloidogenic protein, the method comprising at least the steps of:
(a) providing, optionally producing one or more potentially amyloidogenic protein binding antibodies or a source thereof;
(b) subjecting said one or more potentially amyloidogenic protein binding antibodies or a source thereof to a binding assay comprising the cyclic compound of the present invention;
(c) identifying and optionally obtaining an antibody (subject antibody) that binds to the cyclic compound; and
(d) formulating the antibody identified and optionally obtained in step (c) or a derivative thereof with a pharmaceutically acceptable carrier.

The source of antibodies is not limited and comprises natural as well as synthetic antibodies obtained, for example from immunized laboratory animal such as a rodent, preferably mouse, most preferably Ig humanized mouse; human blood or a fraction thereof preferably comprising memory B cells; recombinant antibody libraries such as phage, yeast, and ribosome systems or mammalian cell systems such as CHO and HEK; see also the “Detailed description of the invention” for further sources of antibodies and other target binding molecules.

The binding assay used in the methods mentioned above preferably comprise ELISA such as performed in Examples 5 and 7.

In a preferred embodiment of the methods of the present invention for identification and obtaining subject antibodies and their further use in being formulated in a pharmaceutical composition and drug development, respectively, the antibody identified and optionally obtained in step (c) competes with a reference antibody for binding the amyloidogenic protein, preferably wherein the subject antibody has a lower EC₅₀ for the amyloidogenic protein than the reference antibody.

Unless defined otherwise in the present application, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. The materials, methods, and examples are illustrative only and not intended to be limiting.

Further embodiments of the present invention will be apparent from the description, Examples and claims that follow. The person skilled in the art will appreciate that every characterization of a generic feature of a general embodiment in the following can and preferably are intended to be combined with the characterization of one or more of the other features of such general embodiment. In addition, unless specifically indicated otherwise, embodiments described herein for antibodies, including the Examples, though being preferred embodiments, are meant as illustration and hereby are extrapolated to any target binding molecule.

### BRIEF DESCRIPTION OF THE DRAWINGS

FIG. 1: NI-301.37F1 3 in vitro phagocytosis assay using human-derived macrophages. NI-301.37F1_3 triggers TTR phagocytosis in a concentration- and FcR-dependent manner. Throughout the description and drawings “NI-301.37F1” may also be referred to as “37F1”. Antibody concentration-dependent TTR uptake was mediated specifically by NI-301.37F1_3, required binding to Fc receptors and low antibody concentration as determined with a standard fluorescence plate reader (A). Quantification of double-positive cells (cells positive for both TTR and NI-301.37F1_3) by FACS showed that the frequency of double-positive cells increased from a background level of 3% to 6% in presence of 10 nM NI-301.37F1_3, and increased further to 16% in presence of 80 nM NI-301.37F1_3 (B). Quantification of cells double positive for TTR and antibody internalized in acidic vesicles showed that the frequency of double-positive cells increased from a basal level of 3.5% to 5.5% in presence of 10 nM NI-301.37F1_3, and increased further to 8.8% in presence of 80 nM NI-301.37F1 3, wherein antibody-dependent phagocytosis of TTR was triggered specifically by NI-301.37F1 3 and not by the isotype control antibody, which did not induce phagocytosis above background level at 10 and 80 nM (C).

FIG. 2: In vitro phagocytosis assay using THP1 cells. NI-301.37F1_3 triggered mis. TTR-488 phagocytosis by THP1 cells in a concentration dependent manner. Quantification of intracellular mis. TTR-488 fluorescence in THP1 cells incubated with 1× or 0.7× NI-301.37F1_3 dilution series (average±SD of triplicates) (A). Both NI-301.37F1 W1 non-GMP DP and NI-301.37F1_W1 GMP DS triggered mis. TTR-488 phagocytosis by THP1 cells in the same concentration range. Quantification of intracellular mis. TTR-488 fluorescence in THP1 cells incubated with NI-301.37F1_W1 non-GMP DP and NI-301.37F1_W1 GMP DS dilution series (average LSD of triplicates) (B)

FIG. 3: Comparison of antibody NI-301.37F1 batch 3 (37F1_3) (A) and NI-301.37F1 batch W1 (37F1_W1) (B) binding to mis. WT-TTR batch 5 and mis. WT-TTR batch 6 using ELISA showed that 37F1_3 and 37F1_W1 binding to mis. WT-TTR_b6 was virtually identical to mis. WT-TTR_b5.

FIG. 4: Evaluation of the ADCP assay of the present invention using mis. WT-TTR as target antigen for its capacity to detect changes in antibody activity by comparison of NI-301.37F1 batch W1 reference sample (NI-301.37F1_W1 RS) and half-concentrated test sample (NI-301.37F1_W1 50%) showed that the assay had the capacity to detect a 50% loss of antibody activity. Mean±SD of triplicates.

FIG. 5: Evaluation of the ADCP assay of the present invention using mis. WT-TTR as target antigen for its capacity to detect changes in antibody activity by comparison of NI-301.37F1_W1 reference sample (NI-301.37F1_W1 RS) with samples with lower (NI-301.37F1_W1 65%, plate 1) (A) and higher (NI-301.37F1_W1 135%, plate 2) (B) concentrations showed that the assay, using a horizontal plate layout had the capacity to detect a 35% loss of antibody activity and a 35% increase in antibody activity, respectively.

FIG. 6: Evaluation of the ADCP assay of the present invention using mis. WT-TTR as target antigen for its capacity to detect changes in antibody activity by comparison of NI-301.37F1_W1 RS, 65% and 135% using a vertical assay layout showed that the assay using the vertical format had the capacity to detect changes in antibody activity by ±35%. Mean±SD of triplicates.

FIG. 7: Binding of stressed NI-301.37F1_W1 samples ((A) reference sample, PBCA pH 3.4, Tris pH 10, H₂O₂; (B) reference sample, Form® buffer pH 5.8, PBS pH 7.4) to mis. WT-TTR was analyzed by ELISA and results showed that stressed NI-301.37F1_W1 samples presented binding affinities for mis. WT-TTR which were highly comparable to the reference NI-301.37F1_W1 sample and characterized by EC₅₀'s in the sub-nanomolar range. (C) Tabular overview of the results.

FIG. 8: Binding of stressed NI-301.37F1_W1 samples to mis. WT-TTR was analyzed by BLI and the tabular overview showed that stressed NI-301.37F1_W1 samples presented binding affinities for mis. WT-TTR which were comparable to the reference NI-301.37F1_W1 sample and characterized by KDs in the low nanomolar range.

FIG. 9: Evaluation of the ADCP assay of the present invention for its capacity to detect potency loss by comparison of NI-301.37F1 W1 RS with NI-301.37F1 W1 samples stressed in PBCA buffer and Tris buffer (A) and with NI-301.37F1_W1 samples stressed in formulation buffer and H₂O₂ buffer (B) showed that the assay had the capacity to detect potency loss related to Fc domain alterations.

FIG. 10: Improved sensitivity of ELISA assay. Comparison of the binding specificity of antibody NI-301.37F1 to (A) peptides TTR34-54cyc, TTR40-49, Biotin. TTR40-49, and mis-WT-TTR, and to (B) peptides TTR34-54cyc, Biotin. TTR34-54cyc, TTR40-49, Biotin. TTR40-49, and mis-WT-TTR using ELISA assays showed specific binding of NI-301.37F1 to mis. WT-TTR, and showed that NI-301.37F1 binding to the cyclic TTR34-54cyc peptide is about 10-fold stronger than binding to mis. WT-TTR. The curve for peptide TTR40-49 is congruent with the one of the Biotin. TTR40-49.

FIG. 11: Improved ADCP assay by use of a cyclic peptide compound as the target antigen. Measuring the potency of antibody NI-301.37F1 in the ADCP assay with a cyclic TTR peptide (TTR.34-54cyc) demonstrates the ability of antibody NI-301.37F1 RS to activate phagocytosis in a dose-response, i.e., in a dose-dependent manner, characterized by an ECso of 19.8 ng/ml.

FIG. 12: ADCP assay using TTR34-54cyc as target antigen for detecting changes in antibody activity by comparison of NI-301.37F1 reference sample (NI-301.37F1 RS) with samples with lower (NI-301.37F1 50% (A) and 70% (B)) concentration, and higher (NI-301.37F1 130% (C) and 150% (D)) concentration showed that the assay had the capacity to detect a 50% loss of antibody activity and a 50% increase in antibody activity, respectively.

### DETAILED DESCRIPTION OF THE INVENTION

The present invention relates to a novel method for determining the potency, in particular the potency to activate antibody-dependent cell-mediated phagocytosis (ADCP), of a target antigen binding molecule comprising an Fc domain as well as to the use of this method in the production and quality control of a pharmaceutical composition comprising such molecule and in the validation of batches of said composition. Furthermore, the present invention relates to a kit which is preferably designed for and can be used in the method of the present invention. In a further aspect, the present invention relates to a cyclic compound which comprises an epitope of a protein recognized by an antibody or equivalent binding molecule, and which can be used as target antigen in the method according to the present invention.

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2; Second edition published 2006, ISBN 0-19-852917-1 978-O-19852917-0.

The term “protein” as used throughout the description includes fragments and peptides of the (full-length) protein, which contain and expose the epitope of the target antigen binding molecule to be tested, for example an antibody.

Reference to the “cyclic peptide” herein can refer to a fully proteinaceous compound, e.g., wherein the linker is 2, 3, 4, 5, 6, 7 or 8 amino acids, or wherein no linker is present. For example, it is possible that the native protein sequence, i.e., amino acid stretch comprising the epitope of the antibody allows cyclization, for example due to the presence of two cysteines in appropriate distance, without addition of extra amino acids. It is understood that properties described for the cyclic peptide determined in the examples can be incorporated in other compounds, e.g., cyclic compounds comprising non-amino acid linker molecules. “Cyclic peptide” and “cyclic compound” can be used interchangeably when the cyclic compound is composed of amino acids.

The term “linker” as used herein means a chemical moiety that can be covalently linked directly or indirectly to the protein fragment or peptide as defined herein. The linker ends can for example be joined to produce a cyclic compound. The linker can be present at a location at the N- and C-termini. Alternatively, the linker may at an internal position at “some distance” from the termini. The linker can comprise one or more functionalizable moieties such as one or more cysteine (C) residues. The linker can be also linked via the functionalizable moieties to other proteins or components. The cyclic compound comprising the linker is of longer length than the peptide or protein fragment itself.

The term “functionalizable moiety” as used herein refers to a chemical entity with a “functional group” which as used herein refers to a group of atoms or a single atom that will react with another group of atoms or a single atom (so called “complementary functional group”) to form a chemical interaction between the two groups or atoms. In the case of cysteine (C), the functional group can be —SH which can be reacted to form a disulfide bond. The reaction with another group of atoms can be covalent or a strong non-covalent bond, for example as in the case as biotin-streptavidin bonds, which can have dissociation constant (Kd) of about 1e-14. A strong non-covalent bond as used herein means an interaction with a Kd of at least 1 e-9, at least 1e-10, at least 1 e-11, at least 1 e-12, at least 1 e-13 or at least 1 e-14.

Potency tests are performed as part of product conformance testing, comparability studies and stability testing. These tests are used to measure product attributes associated with product quality and manufacturing controls, and are performed to assure identity, purity, strength (potency), and stability of products used during all phases of clinical study. Similarly, potency measurements are used to demonstrate that only product lots, i.e., batches that meet defined specifications or acceptance criteria are administered during all phases of clinical investigation and following market approval. Potency is defined as “the specific ability or capacity of the product, as indicated by appropriate laboratory tests or by adequately controlled clinical data obtained through the administration of the product in the manner intended, to effect a given result”. Ideally, the potency assay will represent the product's mechanism of action (i.e., relevant therapeutic activity or intended biological effect); see Guidance for Industry-Potency Tests for Cellular and Gene Therapy Products, U.S. Department of Health and Human Services, Food and Drug Administration, Center for Biologics Evaluation and Research, January 2011. In terms of the assay of the present invention, “potency” of a target antigen binding molecule, specifically an antibody as a drug product is thus a measure of its activity in the ADCP assay relative to the activity of a reference standard (of the drug product) for which the activity and level of activity, respectively, in the ADCP assay has been assessed or is known. Accordingly, a higher potency of the antibody/drug product in comparison to the reference means that the antibody/drug product shows a higher binding activity in the ADCP assay, i.e., a lower EC₅₀ value, and a lower potency of the antibody/drug product in comparison to the reference means that the antibody/drug product shows a lower binding activity in the ADCP assay, i.e., a higher EC₅₀ value. For example, NI-301.37F1 150% mimics an antibody with a higher potency and showed 0.7 times higher EC₅₀ value than the reference sample NI-301.37F1 RS (100%). In contrast antibody NI-301.37F1 50% mimics an antibody with a lower potency (loss of activity) and showed 2 times higher EC₅₀ value than the reference sample NI-301.37F1 RS (100%); see Example 6. Accordingly, a target antigen binding molecule (e.g., an antibody) that exhibits an increase in potency is one that is determined to have, for example, a lower EC₅₀ value relative to a reference sample of at least 1%, e.g., at least 5%, such as at least 10%, or greater (e.g., at least 20%, 30%, 40%, 50%, 60%, 70%, 80%, 90%, or more), for example, as determined in the ADCP assay described herein. Alternatively, a target antigen binding molecule (e.g., an antibody) that exhibits a decrease in potency is one that is determined to have, for example, a higher EC₅₀ value relative to a reference sample of at least 1%, e.g., at least 5%, such as at least 10%, or greater (e.g., at least 20%, 30%, 40%, 50%, 60%, 70%, 80%, 90%, or more), for example, as determined in the ADCP assay described herein. However, according to GLP and GMP, the acceptable variability (i.e., imprecision) for potency measurements is +/−20% which are thus the preferred limits for the tested target antigen binding molecule.

As mentioned above, determining the potency of a drug is an important step in the development including evaluation of new therapeutics for the treatment of diseases. In the context of the present invention, such a method is used for the development, evaluation and batch release of antibody-based drugs and other target antigen binding molecule which make use of the effector function of the Fc domain for the treatment of diseases related to the target protein, especially protein aggregation disorders such as systemic and localized amyloidosis.

The physiological functions of proteins are highly dependent on their correct three-dimensional conformation. Disturbances in the proper folding of newly synthesized or pre-existing proteins as well as in pathways responsible for refolding (molecular chaperones) or degradation of misfolded proteins (ubiquitin-proteasome and autophagy systems) may lead to intra- and/or extracellular protein aggregation. These precipitates of misfolded proteins form either ordered (e.g., amyloid fibrils) or disordered (e.g., inclusion bodies) protein aggregates that dissociate only in the presence of high concentrations of detergents or denaturing buffers (Schröder, Acta Neuropathol 125 (2013), 1-2).

Amyloid diseases are characterized by the deposition of cross-β-sheet amyloid fibrils consisting of misfolded and/or misassembled proteins. The amyloid fibrils that are the pathological hallmark of these disorders can be either deposited systemically or localized to specific organs. The development of amyloidosis is often linked to ageing and is associated with a decreased quality of life and substantial suffering for both patients and their families. Alzheimer's disease is an example of a localized cerebral amyloidosis, and type 2 diabetes mellitus is an example of localized extracerebral amyloidosis; both diseases are associated with ageing. Systemic forms of amyloid disease, also often linked to ageing, are less common and include the TTR amyloidoses. The origin of amyloidosis is either sporadic, i.e., from the normal protein sequence, or hereditary (familial), i.e., from a protein harboring one or more point mutations. In addition, there are infectious forms of amyloidosis, such as the transmissible spongiform encephalopathies caused by the aggregation of prion protein (Ankarcrona et al., J Intern Med. 280 (2016), 177-202).

The present invention provides a reliable method for determining the potency of antibodies and antibody-based drugs in terms of their capability to activate an Fc domain/receptor mediated effector function such as antibody-dependent cell-mediated phagocytosis (ADCP), wherein the antibody preferably targets an epitope on a pathological protein aggregate or an epitope of a cyclic peptide, which is preferably an epitope which is usually exposed in the pathological protein aggregate.

However, as mentioned before, in principle the method of the present invention is applicable to any kind of target antigen, in particular any protein that in its pathogenic variant forms a neoepitope, and any protein fragment or peptide, preferably in cyclic form, that comprises such neoepitope, respectively; see supra.

Thus, in its broadest aspect, the present invention relates to a method for determining the potency of a target antigen binding molecule which comprises an Fc domain comprising the steps of:
(a) contacting a target antigen with the binding molecule under conditions allowing the formation of a binding molecule-target antigen complex;
(b) contacting the binding molecule-target antigen complex with a population of effector cells that are engineered to express an Fc receptor and harbor a reporter gene under the control of a response element that is responsive to activation by the Fc receptor under conditions allowing for binding of the Fc domain to the Fc receptor, wherein binding of the Fc domain to the Fc receptor results in intracellular signaling and mediates a quantifiable reporter gene activity; and
(c) detecting a signal induced by the reporter gene activity, wherein at least one mechanism of action of the Fc domain of the binding molecule is mediated through the binding of the Fc domain to a Fc receptor and the reporter gene activity is indicative for the potency of the binding molecule.

As mentioned above, the method of the present invention is applicable to any target antigen, in particular any protein that in its pathogenic variant forms a neoepitope, for example an epitope which is only exposed in the misfolded variant, a conformational epitope on aggregates, fibrils and/or oligomers, an epitope on extracellular variant of an otherwise physiological protein that is located intracellularly, or an epitope specific for exogenous pathogens such as fungi, bacteria, and viruses. In addition, the method of the present invention in principle can be performed with any kind of antigen including aggregates, fibrils, oligomers, (misfolded) monomers as well as protein fragments and peptides that contain and display the epitope(s) of the target antigen binding molecule to be tested. Thus, in accordance with the method of the present invention, the target antigen is preferably a protein, more preferably an extracellular protein, even more preferred a protein aggregate and fibril, respectively, or an (misfolded) oligomer, a proto-fibril, or (misfolded) monomer, even more preferred an amyloidogenic protein, preferably an amyloidogenic protein in systemic amyloidosis and most preferred TTR and aggregates thereof.

As mentioned above, the protein also includes corresponding fragments and peptides, which contain the (neo) epitope of the target antigen binding molecule. Accordingly, in another preferred embodiment of the method of the present invention, the target antigen is a protein fragment or peptide which comprises an epitope recognized by the target antigen binding molecule. In other words, the target antigen as used in accordance with the method of the present invention is a protein fragment or peptide which is derived from a protein, more preferably from an extracellular protein, even more preferred from a protein which is capable for forming an aggregate and fibril, respectively, or an (misfolded) oligomer, a proto-fibril, or (misfolded) monomer, even more preferred from an amyloidogenic protein, preferably from an amyloidogenic protein in systemic amyloidosis and most preferred from TTR and aggregates thereof, wherein the protein fragment or peptide comprises the epitope of such protein which is recognized by the target antigen binding molecule.

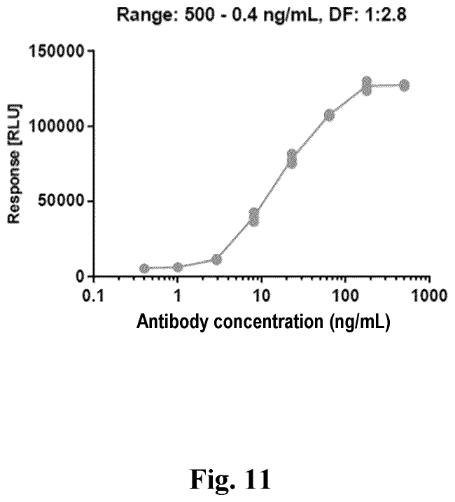

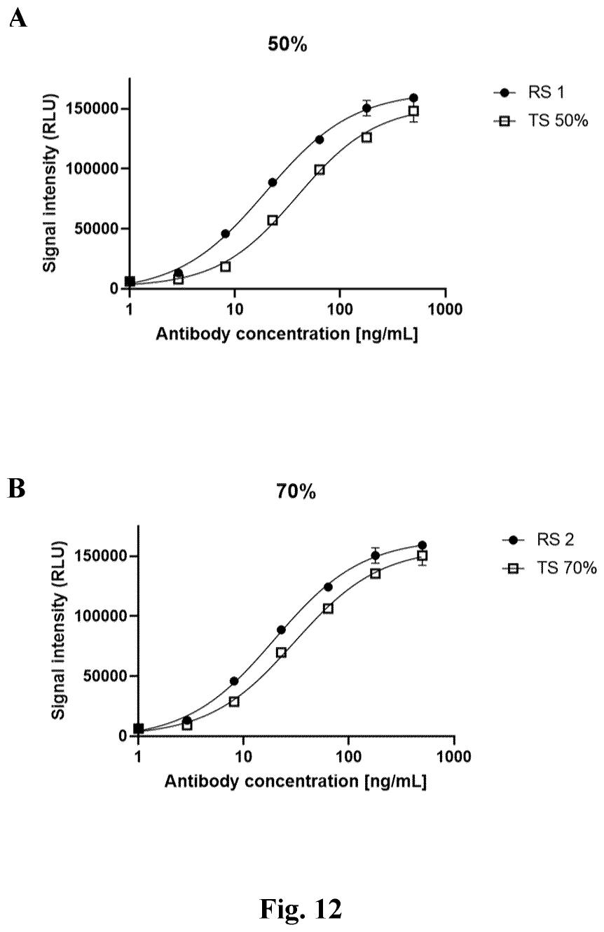

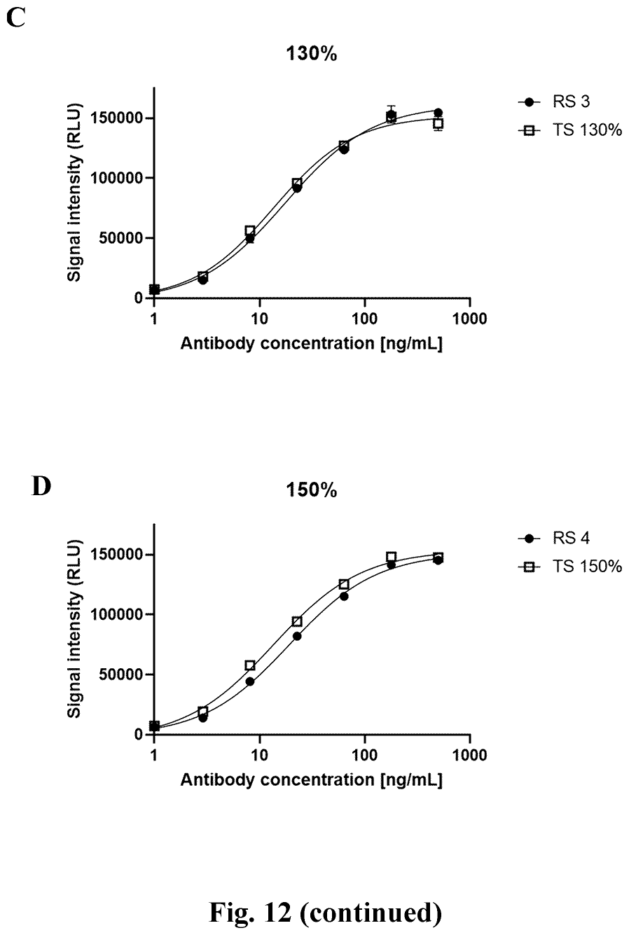

In one embodiment of the method of the present invention, the target antigen comprises or consists of a protein fragment or peptide which contains the (neo) epitope of the target antigen binding molecule. As illustrated in Example 6 and FIGS. 11 and 12, the potency assay of the present invention, i.e., here ADCP assay could be substantially improved by using a cyclic compound comprising the peptide which contains the (neo) epitope of the target antigen binding molecule, here of an anti-TTR antibody. Accordingly, in a preferred embodiment of the method of the present invention, the protein fragment or peptide is cyclized and forms a cyclic compound, respectively; see also supra. The cyclic compound is characterized as described in the preceding section “Summary of the invention” hereinbefore and further below in the sections referring to the cyclic compound per se.

The cyclic compound provided herein and as used in accordance with the present invention can either comprise or consist of a protein fragment or peptide which consists of the epitope recognized by the target antigen binding molecule, or which comprises the epitope recognized by the target antigen binding molecule, meaning that additional amino acids or other chemical entities used for example for cyclization of the peptide or protein fragment as described further below can be present in the protein fragment or peptide which forms the cyclic compound.

The additional amino acids can be amino acids which are naturally located adjacent to the epitope sequence, i.e., amino acids that are flanking the epitope sequence, and which are present in the protein sequence the protein fragment or peptide is derived from, i.e., the protein fragment or peptide which forms the cyclic compound comprises an epitope of the target antigen binding molecule and further amino acids that are adjacent to the epitope and that are flanking the epitope, respectively. The number of those adjacent/flanking amino acids can vary and can be, for example, between 1, 2, or 3 amino acid and 50 amino acids, preferably between 1, 2, or 3 amino acids and 40 amino acids, more preferably between 1, 2, or 3 and 30 amino acids, more preferably between 1, 2, or 3 and 20 amino acids, more preferably between 10 and 20 amino acids, wherein the amino acids are either distributed equally N-terminal and C-terminal to the epitope sequence or unequally, with for example 7 additional amino acids N-terminal and 9 amino acids C-terminal to the epitope.

In addition, or alternatively, the protein fragment or peptide comprises in one embodiment a linker, i.e., the protein fragment or peptide can either comprise the epitope recognized by the target antigen binding molecule without any adjacent amino acids, and a linker, or can comprise the epitope and the adjacent amino acids as defined above, and a linker. In a preferred embodiment, the protein fragment or peptide which forms the cyclic compound as used in accordance with the present invention comprises the epitope which is recognized by the target antigen binding molecule as well as amino acids adjacent to the epitope, and a linker. Preferably, the linker is covalently coupled directly or indirectly to the N-terminus residue of the protein fragment or peptide and to the C-terminal residue of the protein fragment or peptide.

Methods for cyclization of peptides are generally known in the art. For example, cyclization can be performed by chemical crosslinking using inter alia chemical scaffolds. Crosslinking requires functional groups and just few protein chemical targets account for the vast majority of crosslinking techniques, e.g., primary amines (—NH₂), wherein this group exists at the N-terminus of each polypeptide chain and in the side chain of lysine residues; carboxyls (—COOH), wherein this group exists at the C-terminus of each polypeptide chain and in the side chains of aspartic acid and glutamic acid; and sulfhydryls (—SH), wherein this group exists in the side chain of cysteine.

Scaffold-based cyclization is one of the most frequently used methods because it can be applied to chemically or biologically synthesized peptides. In general, scaffold compounds such as organohalides (most frequently organobromides) selectively react with the sulfhydryl group of cysteine. Non-sulfhydryl groups, such as the primary amine of lysine or N-terminal amino group in a peptide, can also be used for cyclization for example with N-hydroxysuccinimide (NHS)-containing chemicals. Especially designed unnatural amino acids can also be used for cyclization in peptides via a bio-orthogonal reaction. For example, if an azide-containing amino acid such as azidohomoalanine or azidophenylalanine exists in a peptide, a copper-mediated click reaction with an alkyne-bearing scaffold can lead to cyclization.

Furthermore, cysteines can be joined together between their side chains via disulfide bonds (—S—S—) or amide cyclization can be performed without any scaffold (head-to-tail, or backbone cyclization).

For example, a peptide with “C” residues at its N- and C-termini, e.g., the cyclic TTR compound used in Examples 5 to 8, GCGGGRKAADDTWEPFASGKTSESGEGGGCG (SEQ ID NO: 17), can be reacted by S—S-cyclization to produce a cyclic peptide. The cyclic compound can be synthesized as a linear molecule with the linker covalently attached at or near the N-terminus or C-terminus of the peptide comprising the TTR peptide, or related epitopes mentioned herein prior to cyclization and provided as a precursor which is also subject of the present invention. Alternatively, part of the linker is covalently attached at or near the N-terminus and part is covalently attached at or near the C-terminus prior to cyclization. In either case, the linear compound is cyclized for example by S—S bond cyclization. Accordingly, the compounds may be cyclized by covalently bonding 1) at or near the N-terminus and the C-terminus of the peptide+linker to form a peptide bond (e.g., cyclizing the backbone), 2) at or near the N-terminus or the C-terminus with a side chain in the peptide+linker, or 3) two side chains in the peptide+linker. In this context, “near” is defined as being within 1, 2, or 3 amino acid residues of the N- or C-terminus. Preferably, the linker is coupled to the N-terminus or C-terminus.

As mentioned above, peptides may be cyclized by oxidation of thiol-or mercaptan-containing residues at or near the N-terminus or C-terminus, or internal to the peptide, including for example cysteine and homocysteine. For example, two cysteine residues flanking the peptide may be oxidized to form a disulphide bond. Oxidative reagents that may employed include, for example, oxygen (air), dimethyl sulphoxide, oxidized glutathione, cystine, copper (II) chloride, potassium ferricyanide, thallium (III) trifluro acetate, or other oxidative reagents such as may be known to those of skill in the art and used with such methods as are known to those of skill in the art. Crosslinking agents are also known in the art and can be chosen for example based on the functional groups to be used for crosslinking, see for example the Crosslinker Selection Tool provided by Thermo Fisher Scientific.

Accordingly, in one embodiment, the linker comprises a functionalizable moiety, e.g., an amino acid with one of the above-mentioned functional groups such as lysine, aspartic acid, glutamic acid, or cysteine, a non-naturally occurring amino acid such as azidohomoalanine or azidophenylalanine, or equivalently functioning molecules such as polyethylene glycol (PEG).

In case the functionalizable moiety is a naturally occurring amino acid, such as lysine, aspartic acid, glutamic acid, serine, threonine, or cysteine, the functionalizable moiety does not necessarily have to be in the linker but can also be present in the epitope or within the adjacent amino acids present in the protein fragment or peptide forming the cyclic peptide. Thus, cyclization of the peptide and the protein fragment, respectively, can also be performed without a linker. Accordingly, in one embodiment, the protein fragment or peptide forms the cyclic compound as used in accordance with the present invention without a linker. The linkage may occur via the side chain of one or more amino acids, such as the sulfhydryl moiety of a cysteine residue, the carboxylic acid moiety of an aspartic acid or glutamic acid residue, the hydroxyl of a serine or threonine residue, or the amine of a lysine or arginine residue.

In a preferred embodiment, the at least one functionalizable moiety is present in the linker, i.e., the linker comprises one or more functionalizable moieties. The linker can comprise or consist of any amino acids including non-natural amino acids, but preferably comprises at least any one of the functionalizable moieties mentioned above, i.e., lysine, aspartic acid, glutamic acid, or cysteine, non-naturally occurring amino acids such as azidohomoalanine or azidophenylalanine, or equivalently functioning molecules such as polyethylene glycol (PEG). In a preferred embodiment, the linker comprises cysteine as functionalizable moiety.

Accordingly, in a preferred embodiment, the linker of any length and sequence can be described with the following sequence X-nX-1FX1-Xn, wherein F is any functionalizable moiety, preferably C (cysteine), and X any amino acid including non-natural amino acids. In a further preferred embodiment, the linker amino acids are selected from alanine (A), or glycine (G), or serine(S), or from alanine (A) and glycine (G), or from glycine (G) and serine(S), but preferably glycine (G).

Even more preferred, the linker amino acids are selected from alanine (A), or glycine (G), or serine(S), or from alanine (A) and glycine (G), or from glycine (G) and serine(S), preferably glycine (G) and the functionalizable moiety is cysteine (C). Accordingly, preferably, the cyclization is performed with scaffold compounds such as organohalides, preferably organobromides, that selectively react with the sulfhydryl group of cysteine, or via a disulfide bridge. Most preferably, cyclization is performed via a disulfide bridge.

In a preferred embodiment, the linker comprises 1 to 40 amino acids, preferably 1 to 35 amino acids, more preferably 1 to 30 amino acids, more preferably 1 to 25 amino acids, more preferably 1 to 20 amino acids, more preferably 1 to 10 amino acids, more preferably 1 to 9 amino acids, and most preferably 1 to 8 amino, in particular 1, 2, 3, 4, 5, 6, 7, or 8 amino acids and/or equivalent functioning molecules, and/or a combination thereof, wherein, when the linker comprises only amino acids, there is preferably within the amino acids at least one amino acid having any of the above-mentioned functional groups, preferably cysteine. The other amino acids comprised in the linker can be chosen from any known amino acids including non-natural amino acids, but are preferably alanine (A) and/or glycine (G), preferably glycine (G).

As mentioned above, the length of the linker can vary and can be for example 9 amino acids, for example GGGGCGGGG (SEQ ID NO: 148), or 8 amino acids, for example GGGCGGGG (SEQ ID NO: 149), GGCGGGGG (SEQ ID NO: 150) or GCGGGGGG (SEQ ID NO: 151), or 7 amino acids, for example GGGGCGG (SEQ ID NO: 152), GGGCGGG (SEQ ID NO: 153), GGCGGGG (SEQ ID NO: 154) or GCGGGGG (SEQ ID NO: 155), 6 amino acids, for example GGGCGG (SEQ ID NO: 156), GGCGGG (SEQ ID NO: 157) or GCGGGG (SEQ ID NO: 158), 5 amino acids, for example GCGGG (SEQ ID NO: 15) or GGGCG (SEQ ID NO: 16), 4 amino acids such as GCGG (SEQ ID NO: 159) or GGCG (SEQ ID NO: 160) or 3 amino acids such as GCG.

Most preferably, the linker in the cyclic compound comprises or consists of GCGGG (SEQ ID NO: 15) or GGGCG (SEQ ID NO: 16).

In a first step of the method of the present invention, the target antigen is provided and contacted with the binding molecule under conditions allowing the formation of a binding molecule-target antigen complex. Different incubation times can be chosen as long as binding of the binding molecule to the target antigen takes place. Thus, incubation conditions can vary, and optimal conditions can be tested. For example, the incubation conditions allowing the binding of the binding molecule to its corresponding antigen might be tested by methods known in the art, for example via ELISA or BLI. Preferably, the incubation time is 30 min and preferably performed at 37° C.

The contacting of the target antigen with the binding molecule can be performed either in solution or by immobilizing the target antigen to a solid support, such as a microplate to which the binding molecule is added.

In one embodiment of the method of the present invention, the target antigen, for example protein aggregate, oligomers, proto-fibrils, fibrils, misfolded monomer or, alternatively a protein fragment or peptide presenting the (neo) epitope of the subject antibody and antibody-based drug, preferably the cyclic compound of the present invention, is contacted with the binding molecule in solution.

In a preferred embodiment of the present invention, the target antigen, for example protein aggregate, oligomers, proto-fibrils, fibrils, misfolded monomer or, alternatively a protein fragment or peptide presenting the (neo) epitope of the subject antibody and antibody-based drug, preferably in form of the cyclic compound, is immobilized on a solid support, preferably on a microtiter plate. In this context, it is understood that the target antigen may be modified, e.g., at or near its C or N terminus for example for the purposes of immobilizing the target antigen on the solid support. In addition, or alternatively, modifications may be made for stabilizing the target antigen, for example for preventing oxidation or otherwise degradation, which are not critical for binding.

The target antigen may be immobilized onto the solid support by common means known in the art and, for example directly coated by hydrophobic interaction without the need for heterologous functional groups such as the biotin-streptavidin system. However, the biotin-streptavidin system can also be used for immobilization.

After the incubation of the binding molecule with the target antigen, a population of engineered effector cells that express an Fc receptor and harbor a reporter gene under the control of a response element that is responsive to the activation by the Fc receptor is added. The binding molecule-target antigen complex is contacted with the effector cells under conditions allowing for binding of the Fc domain of the target antigen binding molecule to the Fc receptors of the effector cells. As mentioned above, different incubation times can be chosen as long as binding of the binding molecule, which is bound to the target antigen, to the effector cells is assured. In a preferred embodiment, the effector cells and the binding molecule-target antigen complex are incubated for about 6 hours at 37° C.

Alternatively, all components, i.e., the target antigen, the binding molecule and the effector cells, can be added simultaneously and co-cultivation leads to binding of the binding molecule to the target antigen and to the Fc receptor on the surface of the effector cells.

The binding of the binding molecule to the Fc receptor results in intracellular signaling which mediates the expression of the reporter gene leading to a quantifiable signal when an appropriate substrate is added. The reporter gene activity is indicative for the potency of the binding molecule meaning that a high reporter gene activity leading to a strong signal is indicative for a high potency of the binding molecule and a low reporter gene activity leading to weak signal is indicative for a low potency of the binding molecule.

Accordingly, there is a strong correlation between the ability of an antibody-based drug product, which is dependent for its mechanism of action on the recruitment of cells expressing the Fc receptor, to bind an Fc receptor, and the therapeutic effect of the drug product when administered to a patient in need thereof.

The potency of a drug product is a measure of the activity in a specific assay relative to the activity of a reference standard of the drug product for which therapeutic efficacy may have been assessed. For binding molecules, such as antibodies, inter alia acting by binding an Fc receptor, a method according to the present invention is suitable for use in determining the potency of the drug product as the binding of the binding molecule to the Fc receptor is a direct indication of a mechanism of action of the binding molecule.

In principle, any reporter gene can be used as long as it confers a detectable signal. For example, any reporter gene can be used that is capable of catalyzing the conversion of a chromogenic, fluorogenic, or chemiluminescent substrate. Such enzymes are known to the person skilled in the art and include for example β-galactosidase, chloramphenicol acetyltransferase, and a luciferase enzyme. In a preferred embodiment of the present invention, a gene is used encoding a bioluminescent protein, preferably a luciferase.

The binding of the Fc domain of the binding molecule to an Fc receptor of the effector cell mediates at least one effector function, i.e., one mechanism of action (MoA) of the Fc domain such as complement-dependent cytotoxicity (CDC), antibody-dependent cellular cytotoxicity (ADCC) and antibody-dependent cell phagocytosis (ADCP).

In one embodiment of the present invention, the MoA is ADCP. It is defined as a highly regulated process by which antibodies eliminate targets via connecting its Fc domain to specific receptors on phagocytic cells and eliciting phagocytosis. In the context of the present invention, ADCP refers to the mechanism(s) by which Fc receptors of phagocytic cells bind to binding molecules, e.g., antibodies that are bound to the target antigen such as aggregated proteins or cyclic compounds comprising the epitope of the proteins of interest and stimulate the phagocytic cells to internalize the protein and the cyclic compound, respectively. However, for the assay of the present invention it is sufficient that the signal to induce ADCP is elicited which leads to reporter gene expression.

The reporter gene used in the method of the present invention is under control of a response element that is responsive to activation by the Fc receptor. Control of gene transcription and translation in response to a stimulus is required to elicit the majority of biological responses such as cellular proliferation, differentiation, survival and immune responses. These non-coding regions of DNA, called response elements, contain specific sequences that are the recognition elements for transcription factors which regulate the efficiency of gene transcription and thus, the amount and type of proteins generated by the cell in response to a stimulus. In a reporter assay, a response element that is responsive to a stimulus is engineered to drive the expression of a reporter gene using standard molecular biology methods. The DNA is then transfected or transduced into a cell, which contains all the machinery to specifically respond to the stimulus, and the level of reporter gene transcription, translation, or activity is measured as a surrogate measure of the biological response.

In one embodiment, the responsive element used in the method of the present invention comprises an NFAT (Nuclear Factor of Activated T cells) response element, AP-1 (Fos/Jun) response element, NF AT/API response element, NFKB response element, FOXO response element, STAT3 response element, STAT5 response element or IRF response element. In some embodiments, the Fc receptor activation responsive elements are arranged as tandem repeats (such as about any of 2, 3, 4, 5, 6, 7, 8, or more tandem repeats). The Fc receptor activation responsive elements may be positioned 5′ or 3′ to the reporter-encoding sequence.

Preferably, the assay of the present invention uses the same ADCP signaling pathway that occurs naturally during phagocytosis. In particular, as with macrophages, the same signaling for ADCP is activated when the binding molecule, which is bound to the target antigen binds to the Fc receptor, meaning that the assay of the present invention reflects the in vivo molecular pathway for Fc receptor-mediated phagocytosis via macrophages. Thus, in a preferred embodiment of the invention, the reporter gene is under control of the nuclear factor of activated T-cells (NFAT) transcription factor.

The effector cells express an Fc receptor. Fc receptors belong to a family of receptors specific for certain amino acids in the constant region of immunoglobulins. Their expression on individual cells depends on the type of receptor. Receptors for almost all immunoglobulin classes have been described. They are referred to as FcγR (for the IgG class), FcaR (for IgA class) and FcER (for IgE class). Thus, in one embodiment of the present invention, the FcR is an FcγR, FcαR, or FcεR family member. Preferably, the effector cells as used in the present invention express an FcγR.

Multiple FcγRs have been identified which differ in their affinity to bind IgG and relative affinity to bind IgG isotypes. Fc receptors for use in the present invention may be full-length Fc receptors or fragments thereof which fragment retains the ability to bind an Fc domain, for instance the extracellular domain. An Fc receptor for use in the present invention may also be a wildtype Fc receptor of any allotype or a mutant variant thereof, the function of which correlates with the function of an Fc receptor, to which the FcR binding molecule binds in vivo. An Fc receptor for use in the present invention may also be a peptide, which is not a naturally occurring Fc receptor (or a fragment or derivate thereof), which peptide is capable of binding the FcR binding region of the Fc part of an antibody and wherein the binding of the FcR binding molecule to the Fc binding peptide correlates with the function of a Fc receptor, to which the FcR binding molecule binds in vivo.

Any Fc receptor can be chosen which is suitable to mediate ADCP, for example FcγRIIa (CD32a), FcγRI (CD64), and FcγRIIIa (CD16a). In a preferred embodiment, the Fc receptor is an FcγR, more preferably FcγRI. Optionally, the effector cells do not express or overexpress FcγRIIa (CD32a) and/or FcγRIIIa (CD16a).

In one embodiment, the effector cells endogenously express the Fc receptor, i.e., the cell comprises an endogenous sequence encoding an Fc receptor, wherein the cell is for example a macrophage, a mast cell, a monocyte, a neutrophil or a dendritic cell.

In another, preferred embodiment, the effector cells have been modified to express an Fc receptor, i.e., the cell is engineered to comprise a heterologous sequence encoding an Fc receptor. In principle, any cell can be used which is suitable to express an Fc receptor. For example, the cell can be a cell selected from the group consisting of 8V-2, THP-1, CHO, 293-T, 3T3, 4T1, 721, 9L, A2780, A172, A20, A253, A431, A-549, ALC, 816, 835, 8CP-1, 8EAS-28, bEnd.3, 8HK-21, 8R293, 8xPC3, C3H-10T1/2, C6, Cal-27, COR-L23, COS-7, CML T1, CMT, CT26, 017, OH82, OU145, OuCaP, EL4, EM2, EM3, EMT6/ARI, FM3, H1299, H69, H854, H855, HCA2, HEK-293, Hela, Hepalele7, HL-60, HMEC, HT-29, HUVEC, Jurkat, J558L, JY, K562, Ku812, KCL.22, KGI, KYOI, MCF-7, R8L, Saos-2, SK8R3, SKOV-3, T2, T-470, T84, U373, U937, Vero, and J774. In a preferred embodiment, the cell is a Jurkat cell.

The method of the present invention determines the potency of a target antigen binding molecule comprising an Fc domain. Such molecule binds to any protein that in its pathogenic variant forms a neoepitope; see supra.

The molecule which potency is assessed with the method of the present invention can be any molecule which is capable of binding to a target antigen. In one embodiment, such a molecule comprises an Fc domain. Preferably, the target antigen binding molecule is an antibody or any fragment, derivative or mimetic thereof which comprises an Fc domain. The target antigen comprises a full-length Fc domain or an FcR-binding fragment of an Fc domain as long as it remains functional.

As used herein, an “antibody” is a glycoprotein comprising at least two heavy (H) chains and two light (L) chains interconnected by disulfide bonds. Each heavy chain comprises a heavy chain variable region (VH) and a heavy chain constant region. The heavy chain constant region comprises three domains, CH1, CH2, and CH3. Each light chain comprises a light chain variable region (VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with a target antigen, e.g., a target protein. The constant regions of the antibodies may mediate the binding of the immunoglobulin to host tissues or factors, including various cells of the immune system (e.g., effector cells) and the first component (Clq) of the classical complement system. The term “antibody” also includes antibody formats that do not contain the entire binding domain of, for example, an IgG antibody, but still bind the target antigen. Such antibody fragments include for example single variable domain antibodies, for example nanobodies which are linked to Fc-domains, in particular chimeric nanobody-heavy chain antibodies which combine advantageous features of nanobodies and Fc domains in about half the size of a conventional antibody (see, e.g., Bannas et al., Front. Immunol. (2017), DOI: 10.3389/fimmu.2017.01603). In general, the term “antibody” encompasses any antibody fragment comprising an Fc domain.

Antibodies may be monoclonal antibodies or polyclonal antibodies. A “monoclonal antibody” refers to a preparation of antibody molecules of single molecular composition and/or obtained from a population of substantially homogenous antibodies. A monoclonal antibody displays a single binding specificity and affinity for a particular epitope. A “polyclonal antibody” refers to a heterogeneous pool of antibodies produced by a number of different B lymphocytes. Different antibodies in the pool recognize and specifically bind different epitopes. An “epitope” refers to a polypeptide sequence that, by itself or as part of a larger sequence, binds to an antibody generated in response to the sequence. A target protein, e.g., TTR may contain linear, discontinuous epitopes, and/or conformational epitopes.

The antibodies can be humanized antibodies. A “humanized antibody” refers to an antibody that retains only the protein-binding CDRs from the parent antibody in association with human framework. In some embodiments, the antibodies are human antibodies. A “human antibody” refers to antibodies having variable and constant regions derived from human germline immunoglobulin sequences or from a human subject. Human antibodies can include amino acid residues not encoded by human germline immunoglobulin sequences (e.g., mutations introduced by random or site-specific mutagenesis in vitro or by somatic mutation in vivo). The term “human antibody,” as used herein, does not include antibodies in which CDR sequences derived from the germline of another mammalian species, such as a mouse have been grafted onto human framework sequences (referred to herein as “humanized antibodies”). Human antibodies can be for example obtained as described in WO 2008/081008 A1. Humanized mice which have become a prominent source for human antibodies against diverse targets which do not or only poorly elicit an immune and memory B cell response. Several transgenic animal platforms are available, for example OmniAb® from Ligand in US, Alloy ATX-GK™ Mouse in US and CAMouse™ from CAMAB in China. For example, the RenMab™ mouse was recently developed which carries the entire human variable region segments of heavy chain and kappa chain. For review of preeminent antibody engineering technologies used in the development of therapeutic antibody drugs, such as humanization of monoclonal antibodies, phage display, the human antibody mouse, single B cell antibody technology, and affinity maturation; see, e.g., Lu et al., J. Biomed. Sci. 27 (2020), doi.org/10.1186/s12929-019-0592-z, and references cited therein.

The antibodies can be chimeric antibodies, for example murine-human, murinized, bispecific or multispecific antibodies or IgGs.

The antibodies can be recombinant antibodies. A “recombinant antibody” refers in general to antibodies that are prepared, expressed, created, and/or isolated by recombinant means. A review on current antibody production systems is given in Frenzel et al., Front Immunol. 4 (2013), 217, DOI: 10.3389/fimmu.2013.00217 and transient expression of human antibodies in mammalian cells is described by Vazquez-Lombardi et al., Nature protocols 13 (2018), 99-117; and Hunter et al., Current Protocols in Protein Science 95 (2019), e77. DOI: 10.1002/cpps. 77. The antibody can be of a specific isotype referring to the immunoglobulin class that is encoded by heavy chain constant region genes, for instance IgG1, IgG2, IgG3, IgG4, IgD, IgA, IgE, or IgM. Each isotype has a unique amino acid sequence and possesses a unique set of isotype epitopes distinguishing them from each other. However, preferably the potency of an IgG, in particular of an IgG1 antibody such as an IgG1, λ antibody or an IgG1, κ antibody is assessed with the method of the present invention.

As indicated above, the term antibody herein, unless otherwise stated or clearly contradicted by context, includes fragments, derivatives, variants (incl. deletion variants) of an antibody that retain the ability to specifically bind to an antigen and to an Fc receptor as well as antibody mimetics.

Further molecules fused to an Fc domain, for example antibody mimetics, can be analyzed with the method of the present invention, which include for example designed ankyrin repeat proteins (DARPins) which are fused with an Fc domain. Further included are for example engineered Fc based antibody domains and fragments, e.g., the dimeric Fc, mFc, CH2 and mCH3 scaffolds into which CDRs are grafted and/or which are engineered in that the loop regions are incorporated at the C-terminal of the CH3 domains of Fc to form new antigen-binding sites (Ying et al., Biochim Biophys Acta. 1844 (2014), 1977-1982, DOI: 10.1016/j.bbapap.2014.04.018 Wozniak-Knopp et al. Protein Eng Des Sel. 23 (2010), 289-297, DOI: 10.1093/protein/gzq005) as well as Fc-fusion proteins which are composed of an immunoglobulin (Ig) Fc domain that is directly linked to another peptide, protein, or protein domain. For therapeutic propose, the first description of CD4-Fc fusion protein showed the inhibitory activity against the formation of syncytia during HIV-1 infection in 1989, which showed the proof-of-concept of use of therapeutic Fc-fusion proteins for treatment of HIV-1 infection (Yang et al. Front Immunol 8 (2018), 1860, DOI: 10.3389/fimmu.2017.01860.

As can be derived from Examples 3 and 4, the assay has been illustrated with the amyloidogenic protein TTR and aggregates thereof, respectively, as the target antigen and an anti-TTR antibody, NI-301.37F1 disclosed in international application WO 2015/092077 A1, but in principle, the method of the present invention can be used for the analysis of any target antigen, in particular any protein that in its pathogenic variant forms a neoepitope, for example an epitope which is only exposed in the misfolded variant, a conformational epitope on aggregates, fibrils and/or oligomers, an epitope on extracellular variant of an otherwise physiological protein that is located intracellularly, or an epitope specific for exogenous pathogens such as fungi, bacteria, and viruses.

Such a protein can in principle be any protein, preferably any protein which aggregation leads to a disease phenotype. Exemplarily proteins include but are not limited to transthyretin (TTR), wherein the TTR is wildtype or mutated TTR, preferably wildtype TTR, α-synuclein (α-syn), tau, prion protein (PrP), amyloid beta (Aβ), β2-microglobulin (β2-m), Immunoglobulin light chain (LC), Immunoglobulin heavy chain (HC), serum amyloid A (SAA), amylin (IAPP), Chromosome 9 open reading frame 72 (C9orf72), TAR DNA-binding protein 43 (TDP-43), superoxide dismutase 1 (SOD1), RNA-binding protein fused in sarcoma (FUS), huntingtin (htt), optineurin (OPTN), neuroserpin, ABri, Adan, ubiquilin, optineurin, leucocyte chemotactic factor 2 (LECT2), gelsolin, apolipoprotein AI (ApoAI), apolipoprotein AII (ApoAII), apolipoprotein AIV (ApoAIV), apolipoprotein CII (ApoCII), apolipoprotein CIII (ApoCIII), fibrinogen, cystatin C, and lysozyme. Further amyloid fibril-forming proteins can be derived from AmyPro, an open-access database providing a collection of amyloid fibril-forming proteins (Varadi et al., Nucleic Acids Research 46 (2018), D387-D392, DOI: 10.1093/nar/gkx950), and/or can be those listed in Table 1 of Benson et al., Amyloid 25 (2018), 215-219.

In a preferred embodiment, the amyloidogenic protein is involved in systemic amyloidosis, and more preferably selected from the following list: transthyretin (TTR), in particular wild type TTR and variant TTR, preferably wild type TTR, immunoglobulin light chain (LC), immunoglobulin heavy chain (LII), serum amyloid A (SAA), leucocyte chemotactic factor 2 (LECT2), gelsolin, apolipoprotein AI (ApoAI), apolipoprotein AII (ApoAII), apolipoprotein AIV (ApoAIV), apolipoprotein CII (ApoCII), apolipoprotein CIII (ApoCIII), fibrinogen, β2 microglobulin, in particular wild type and variant β2 microglobulin, cystatin C, ABriPP, prion protein, and lysozyme; see for example Benson et al., Amyloid 25 (2018), 215-219 and Muchtar et al., Journal of Internal Medicine 289 (2021), 268-292.

As illustrated in Example 6, the assay has been successfully performed with a cyclic peptide compound which comprises the epitope WEPFA of antibody NI-301.37F1 disclosed in international application WO 2015/092077 A1, which is a neoepitope in the sense that is located in position 41-45 of mature TTR protein, which is hidden in the TTR protein's naturally folded conformation but accessible to antibody binding following unfolding and aggregation, as the target antigen and the anti-TTR antibody NI-301.37F1 as the target antigen binding molecule.

Thus, in one preferred embodiment, the target antigen, i.e., the protein fragment or peptide preferably in the form of a cyclic compound, comprises a (neo) epitope, preferably from any protein which aggregation leads to a disease phenotype. Preferably, the (neo) epitope is derived from an amyloidogenic protein involved in systemic amyloidosis or aggregate thereof.

The protein fragment or peptide in the cyclic compound of the present invention and as used in the potency assay comprises at least 4, preferably at least 5, more preferably at least 10, more preferably at least 15, most preferably at least 20, 21, 22, 23, 24, or 25 amino acid residues of an amyloidogenic protein. More specifically, at least the epitope of the target antigen binding molecule, which as known to the person skilled in the art can consist of as few amino acids as four should be present, which may be supplemented with appropriate number of amino acids and/or other linker moieties sufficient and necessary for cyclization.

However, in principle there is no limitation as to length of the peptide as long as it can be cyclized, and it is recognized by the target binding molecule. Accordingly, the cyclic compound of the present invention and as used herein may comprise a protein or fragment or peptide thereof containing between 4 amino acids and all amino acids of the amyloidogenic protein. Preferably, the protein fragment or peptide in the cyclic compound comprises between 4 amino acids and 100 amino acids, more preferably between 4 amino acids and 90 amino acids, more preferably between 4 amino acids and 80 amino acids, more preferably between 4 amino acids and 70 amino acids, more preferably between 4 amino acids and 60 amino acids, more preferably between 4 amino acids and 50 amino acids, more preferably between 4 amino acids and 45 amino acids, more preferably between 4 amino acids and 40 amino acids, more preferably between 4 amino acids and 35 amino acids, more preferably between 4 amino acids and 30 amino acids, more preferably between 4 amino acids and 25 amino acids, or between 4 amino acids and 24 amino acids, or between 4 amino acids and 23 amino acids, or between 4 amino acids and 22 amino acids, or between 4 amino acids and 21 amino acids, or between 4 amino acids and 20 amino acids, preferably between 5 amino acids and 25 amino acids, or between 5 amino acids and 24 amino acids, or between 5 amino acids and 23 amino acids, or between 5 amino acids and 22 amino acids, or between 5 amino acids and 21 amino acids, or between 5 amino acids and 20 amino acids.

The amino acids either represent only the epitope recognized by a target antigen binding molecule or the epitope and adjacent amino acids present in the amyloidogenic protein. In a preferred embodiment, the protein fragment of peptide comprises amino acid residues of an amyloidogenic protein, wherein these amino acid residues comprise the epitope and adjacent amino acids.

The cyclic TTR peptide used in the Examples 5 and 6 consists of the amino acid sequence H-GCGGGRKAADDTWEPFASGKTSESGEGGGCG-OH (TTR34-54cyc; SEQ ID NO: 17) with a total of 31 amino acids and comprises 21 amino acids of the amyloidogenic protein TTR, including the five amino acid epitope WEPFA, and linker sequences of 10 amino acids, five amino acids each the N- and C-termini of the 21 amino acid stretch from TTR. Thus, in a preferred embodiment, the cyclic compound consists of a total of 20 to 40, more preferably 25 to 35 and most preferably of 30±one, two, three or four amino acids or, in case non-amino acid residues are incorporated, for example as a linker, is configured such that its structure resembles a corresponding peptide. In this embodiment, the amino acid sequence derived from the amyloidogenic protein present in the cyclic compound may consist of 10 to 40, preferably of 15 to 25 and most preferably of 20 ±one, two, three or four amino acids and optionally supplemented with a linker, preferably 5 to 20 amino acids in length, more preferably 5 to 15 and most preferably of 10±one, two, three or four amino acids, either distributed on both ends, N- and C-terminus or only at one terminus. It is also conceivable that linker sequences or “filling” sequences are located within amino acid sequence derived from the amyloidogenic protein, e.g., if the epitope of the target binding molecule is a conformational epitope or discontinuous epitope.

As mentioned above, the method is in general applicable to any target antigen, but preferably, the protein fragment or peptide is derived from an amyloidogenic protein and comprises a (neo) epitope of the target antigen binding molecule. The amyloidogenic protein can in principle be any amyloidogenic protein as for example listed in Table 1 of Benson et al., Amyloid 25 (2018), 215-219 and as mentioned above. In a preferred embodiment, the amyloidogenic protein is involved in systemic amyloidosis, and more preferably selected from the following list: transthyretin (TTR), in particular wild type TTR and variant TTR, immunoglobulin light chain (LC), immunoglobulin heavy chain (LH), serum amyloid A (SAA), leucocyte chemotactic factor 2 (LECT2), gelsolin, apolipoprotein AI (ApoAI), apolipoprotein AII (ApoAII), apolipoprotein AIV (ApoAIV), apolipoprotein CII (ApoCII), apolipoprotein CIII (ApoCIII), fibrinogen, B2 microglobulin, in particular wild type and variant β2 microglobulin, cystatin C, ABriPP, prion protein, and lysozyme, and thus, the target antigen comprises a peptide derived from any one of the listed proteins, preferably wherein the peptide comprises at least 4 amino acids from the protein.

In a preferred embodiment, the amyloidogenic protein is TTR and thus, the target antigen comprises a protein fragment of or peptide derived from TTR.

In general, the protein fragment or peptide of TTR can be any fragment or peptide that is derived from the TTR protein. In a preferred embodiment, the TTR fragment or peptide in the cyclic compound used in accordance with the method of the present invention comprises at least 4 amino acids from the TTR protein, wherein the 4 amino acids can for example be any one of those listed in Table 1, below.

TABLE 1
TTR peptides comprising 4 amino acid residues.

| | | | |
|---|---|---|---|
| GPTG | PTGT | TGTG | GTGE |
| (SEQ ID NO: 24) | (SEQ ID NO: 55) | (SEQ ID NO: 86) | (SEQ ID NO: 117) |
| TGES | GESK | ESKC | SKCP |
| (SEQ ID NO: 25) | (SEQ ID NO: 56) | (SEQ ID NO: 87) | (SEQ ID NO: 118) |
| KCPL | CPLM | PLMV | LMVK |
| (SEQ ID NO: 26) | (SEQ ID NO: 57) | (SEQ ID NO: 88) | (SEQ ID NO: 119) |
| MVKV | VKVL | KVLD | VLDA |
| (SEQ ID NO: 27) | (SEQ ID NO: 58) | (SEQ ID NO: 89) | (SEQ ID NO: 120) |
| LDAV | DAVR | AVRG | VRGS |
| (SEQ ID NO: 28) | (SEQ ID NO: 59) | (SEQ ID NO: 90) | (SEQ ID NO: 121) |
| RGSP | GSPA | SPAI | PAIN |
| (SEQ ID NO: 29) | (SEQ ID NO: 60) | (SEQ ID NO: 91) | (SEQ ID NO: 122) |
| AINV | INVA | NVAV | VAVH |
| (SEQ ID NO: 30) | (SEQ ID NO: 61) | (SEQ ID NO: 92) | (SEQ ID NO: 123) |
| AVHV | VHVF | HVFR | VFRK |
| (SEQ ID NO: 31) | (SEQ ID NO: 62) | (SEQ ID NO: 93) | (SEQ ID NO: 124) |
| FRKA | RKAA | KAAD | AADD |
| (SEQ ID NO: 32) | (SEQ ID NO: 63) | (SEQ ID NO: 94) | (SEQ ID NO: 125) |
| ADDT | DDTW | DTWE | TWEP |
| (SEQ ID NO: 33) | (SEQ ID NO: 64) | (SEQ ID NO: 95) | (SEQ ID NO: 126) |
| WEPF | EPFA | PFAS | FASG |
| (SEQ ID NO: 34) | (SEQ ID NO: 65) | (SEQ ID NO: 96) | (SEQ ID NO: 127) |
| ASGK | SGKT | GKTS | KTSE |
| (SEQ ID NO: 35) | (SEQ ID NO: 66) | (SEQ ID NO: 97) | (SEQ ID NO: 128) |
| TSES | SESG | ESGE | SGEL |
| (SEQ ID NO: 36) | (SEQ ID NO: 67) | (SEQ ID NO: 98) | (SEQ ID NO: 129) |
| GELH | ELHG | LHGL | HGLT |
| (SEQ ID NO: 37) | (SEQ ID NO: 68) | (SEQ ID NO: 99) | (SEQ ID NO: 130) |
| GLTT | LTTE | TTEE | TEEE |
| (SEQ ID NO: 38) | (SEQ ID NO: 69) | (SEQ ID NO: 100) | (SEQ ID NO: 131) |
| EEEF | EEFV | EFVE | FVEG |
| (SEQ ID NO: 39) | (SEQ ID NO: 70) | (SEQ ID NO: 101) | (SEQ ID NO: 132) |
| VEGI | EGIY | GIYK | IYKV |
| (SEQ ID NO: 40) | (SEQ ID NO: 71) | (SEQ ID NO: 102) | (SEQ ID NO: 133) |
| YKVE | KVEI | VEID | EIDT |
| (SEQ ID NO: 41) | (SEQ ID NO: 72) | (SEQ ID NO: 103) | (SEQ ID NO: 134) |
| IDTK | DTKS | TKSY | KSYW |
| (SEQ ID NO: 42) | (SEQ ID NO: 73) | (SEQ ID NO: 104) | (SEQ ID NO: 135) |
| SYWK | YWKA | WKAL | KALG |
| (SEQ ID NO: 43) | (SEQ ID NO: 74) | (SEQ ID NO: 105) | (SEQ ID NO: 136) |
| ALGI | LGIS | GISP | ISPF |
| (SEQ ID NO: 44) | (SEQ ID NO: 75) | (SEQ ID NO: 106) | (SEQ ID NO: 137) |
| SPFH | PFHE | FHEH | HEHA |
| (SEQ ID NO: 45) | (SEQ ID NO: 76) | (SEQ ID NO: 107) | (SEQ ID NO: 138) |
| EHAE | HAVE | AEVV | EVVF |
| (SEQ ID NO: 46) | (SEQ ID NO: 77) | (SEQ ID NO: 108) | (SEQ ID NO: 139) |
| VVFT | VFTA | FTAN | TAND |
| (SEQ ID NO: 47) | (SEQ ID NO: 78) | (SEQ ID NO: 109) | (SEQ ID NO: 140) |
| ANDS | NDSG | DSGP | SGPR |
| (SEQ ID NO: 48) | (SEQ ID NO: 79) | (SEQ ID NO: 110) | (SEQ ID NO: 141) |
| GPRR | PRRY | RRYT | RYTI |
| (SEQ ID NO: 49) | (SEQ ID NO: 80) | (SEQ ID NO: 111) | (SEQ ID NO: 142) |
| YTIA | TIAA | IAAL | AALL |
| (SEQ ID NO: 50) | (SEQ ID NO: 81) | (SEQ ID NO: 112) | (SEQ ID NO: 143) |
| ALLS | LLSP | LSPY | SPYS |
| (SEQ ID NO: 51) | (SEQ ID NO: 82) | (SEQ ID NO: 113) | (SEQ ID NO: 144) |
| PYSY | YSYS | SYST | YSTT |
| (SEQ ID NO: 52) | (SEQ ID NO: 83) | (SEQ ID NO: 114) | (SEQ ID NO: 145) |
| STTA | TTAV | TAVV | AVVT |
| (SEQ ID NO: 53) | (SEQ ID NO: 84) | (SEQ ID NO: 115) | (SEQ ID NO: 146) |
| VVTN | VTNP | TNPK | NPKE |
| (SEQ ID NO: 54) | (SEQ ID NO: 85) | (SEQ ID NO: 116) | (SEQ ID NO: 147) |

In a preferred embodiment, the TTR peptide comprises at least 4 amino acid residues and preferably all amino acids of an amino acid sequence which is exposed in the misfolded variant, and on aggregates, fibrils and/or oligomers, respectively, e.g., WEPFA (SEQ ID NO: 1), which is a peptide recognized by antibody NI-301.37F1 or by antibody NI-301.28B3 disclosed in WO 2015/092077 A1; EEFXEGIY (SEQ ID NO: 2), which is a peptide recognized for example by antibody NI-301.59F1 disclosed in WO 2015/092077 A1; ELXGLTXE (SEQ ID NO: 3), which is a peptide recognized for example by antibody NI-301.35G11 disclosed in WO 2015/092077 A1, wherein X can be any amino acid; WEPFASG (SEQ ID NO: 4), which is a peptide recognized for example by antibody NI-301.12D3 disclosed in WO 2015/092077 A1; TTAVVTNPKE (SEQ ID NO: 5), which is a peptide recognized for example by antibody NI-301.18C4 disclosed in WO 2015/092077 A1; KCPLMVK and VFRK (SEQ ID NOs: 6 and 7), which represent peptides comprising a conformational epitope requiring at least the C of the first sequence and the V and F of the second sequence, and which is an epitope recognized by antibody NI-301.44E4 of WO 2015/092077 A1; EHAEVVFTA (SEQ ID NO: 8), which is a peptide recognized for example by antibody 14G8/PRX004 disclosed inter alia in Higaki et al., Amyloid 23 (2016), 86-97; GPRRYTIAA (SEQ ID NO: 9), which is a peptide recognized for example by antibody 18C5 described in WO 2019/071205 A1; VHVFRKAADDTWEPFASGKTSESGELHGLTTEEEFVE (SEQ ID NO: 10), which is a peptide recognized for example by an antibody described in WO 2014/124334 A2 which binds to TTR30-66; ALLSPYSYSTTAV (SEQ ID NO: 11), which is a peptide recognized for example by an antibody described in WO 2014/124334 A2 which binds to TTR109-121; WKALGISPFHE (SEQ ID NO: 12), which is a peptide recognized for example by antibody 371M described in WO 2015/115332 A1; SYSTTAVVTN (SEQ ID NO: 13), which is a peptide recognized for example by antibody 313M (RT24) described in WO 2015/115331 A1; or LLSPYSYSTTAVVTNPKE (SEQ ID NO: 14), which is a peptide recognized for example by an antibody described in WO 2014/124334 A2 which binds to TTR100-127.

Most preferably, the TTR peptide in accordance with the method of the present invention comprises the amino acid sequence WEPFA (SEQ ID NO: 1)

As mentioned above, the cyclic compound used in accordance with the method of the present invention comprises preferably a protein fragment or peptide comprising an epitope of an amyloidogenic protein, preferably a TTR epitope, and most preferably the epitope comprising the amino acid sequence WEPFA (SEQ ID NO: 1) as well as adjacent amino acids and a linker at the peptide N-terminus and C-terminus, wherein the linker can in principle comprise any of the above-described linker sequences, and preferably comprises the amino acid sequence GCGGG (SEQ ID NO: 15) or GGGCG (SEQ ID NO: 16). Thus, a preferred embodiment of the method of the present invention, the cyclic compound comprises or consists of the amino acid sequence H-GCGGGRKAADDTWEPFASGKTSESGEGGGCG-OH (TTR34-54cyc; SEQ ID NO: 17), which has been shown in Examples 5 and 6 as suitable target antigen.

Accordingly, the binding molecule which potency, in particular its potency to induce ADCP, is determined with the method of the present invention can be any binding molecule which binds to said target antigen, preferably any protein in its pathogenic variant which induces a disease phenotype and a corresponding protein fragment or peptide thereof. Exemplarily antibodies include but are not limited to anti-TTR antibodies, anti-α-syn antibodies, anti-tau antibodies, anti-PrP antibodies, anti-AB antibodies, anti-β2-m antibodies, anti-LC antibodies, anti-HC antibodies, anti-SAA antibodies, anti-IAPP antibodies, anti-C9orf72 antibodies, anti-TDP-43 antibodies, anti-SOD1 antibodies, anti-FUS antibodies, anti-htt antibodies, anti-OPTN antibodies, anti-neuroserpin antibodies, anti-ABri antibodies, anti-ADan antibodies, anti-ubiquilin antibodies, anti-optineurin antibodies, anti-LECT2 antibodies, anti-gelsolin antibodies, anti-ApoAI antibodies, anti-ApoAII antibodies, anti-ApoAVI antibodies, anti-ApoCII antibodies, anti-ApoCIII antibodies, anti-fibrinogen antibody, anti-cystatin C antibodies, anti-ABriPP antibodies, anti-prion antibodies, and anti-lysozyme antibodies.

In a preferred embodiment, the binding molecule is a binding molecule binding to targets involved in systemic amyloidosis and thus, the antibody is preferably selected from the group consisting of: anti-TTR antibody, anti-LC antibody, anti-HC antibody, anti-SAA antibody, anti-LECT2 antibody, anti-gelsolin antibody, anti-ApoAI antibody, anti-ApoAII antibody, anti-ApoAVI antibody, anti-ApoCII antibody, anti-ApoCIII antibody, anti-fibrinogen antibody, anti-β2 microglobulin antibody, anti-cystatin C antibody, anti-ABriPP antibody, anti-prion antibody, and anti-lysozyme antibody.

Thus, the assay of the present invention may be used to measure the activity/potency of any suitable binding molecule. Suitable antibodies are known in the art, but in the following exemplarily antibodies are listed.

Anti-TTR antibodies, which are the preferred ones to be analyzed with the method of the present invention, may include those disclosed in WO 2015/092077 A1, in particular antibodies being characterized by binding a human TTR epitope which comprises or consists of the amino acid sequence TTR41-45 (SEQ ID NO: 51 of WO 2015/092077 A1), in particular NI-301.37F1, NI-301.28B3, and NI-301.12D3. Furthermore, PRX004, which is currently in a Phase 1 study in patients with ATTR (ClinicalTrials.gov Identifier: NCT03336580) may be a suitable antibody. Antibody PRX004 corresponds to and is the humanized version of mouse monoclonal antibody 14G8 described in Higaki et al., Amyloid 23 (2016) 86-97 (see WO 2019/071206 A1 at page 91 in Table 4) and which is described in WO 2016/120810 A1 and WO 2018/007922A2 and more specifically in WO 2019/108689 A1. Further suitable antibodies are antibodies which recognize the same epitope as antibody PRX004 i.e., amino acids TTR89-97 or an epitope comprising amino acids TTR101-109, and which are humanized versions of the originally cloned mouse monoclonal antibodies 14G8, 9D5, 5A1, 6C1 described in WO 2016/120810 A1, WO 2018/007924 A2, WO 2018/007924 A2 and WO 2018/007923 A1. Further suitable antibodies are a humanized version of antibody 18C5 as described in WO 2019/071205 A1, antibody 371M having an epitope at positions 79-89 of human TTR described in WO 2015/115332 A1 and antibody 313M (RT24) having an epitope within TTR115-124 positions of human TTR described in WO 2015/115331 A1. These antibodies can also be used as control antibodies in the method of the present invention.

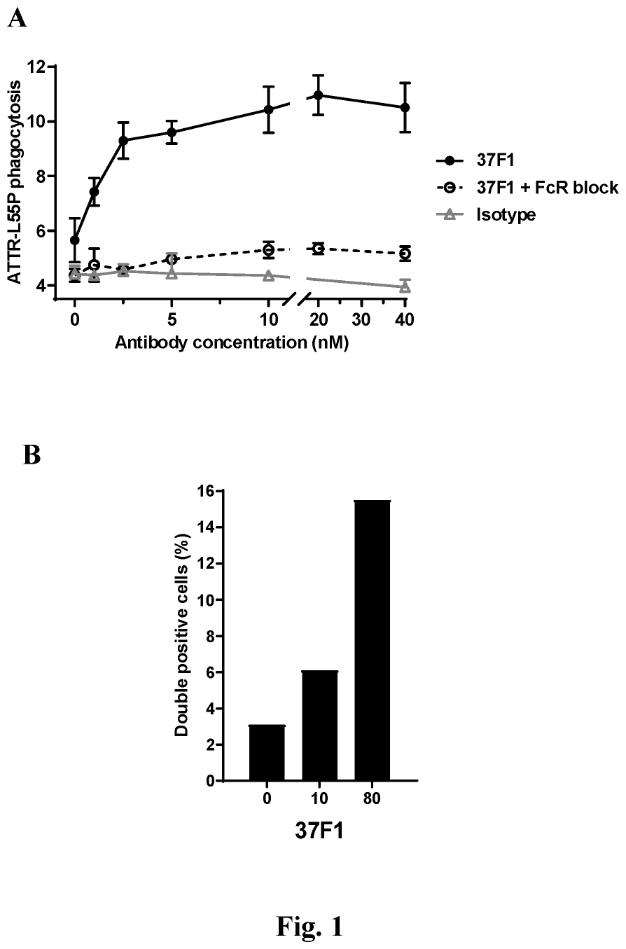

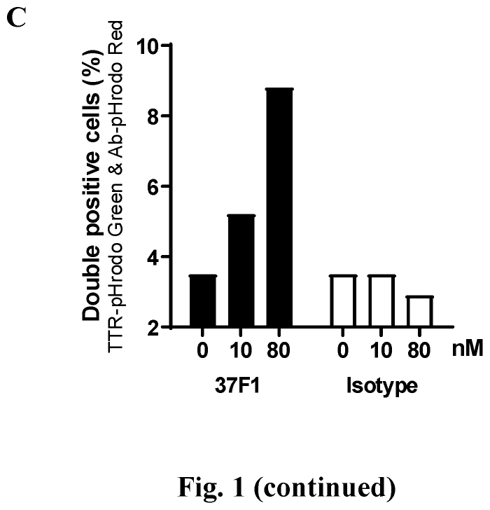

In a preferred embodiment, the binding molecule is the anti-TTR antibody NI-301.37F1 which is characterized by comprising in its variable region, i.e., binding domain the complementarity determining regions (CDRs) of the variable heavy (V_{H}) and variable light (V_{L}) chain having the amino acid sequences depicted in FIG. 1C of WO 2015/092077 A1 and shown in present Table 2, or wherein one or more of the CDRs may differ in their amino acid sequence from those set forth in FIG. 1C of WO 2015/092077 A1 and in present Table 2 by one, two, three or even more amino acids in case of CDR2 and CDR3, and wherein the antibody displays substantially the same or identical characteristics of anti-TTR antibody NI-301.37F1 illustrated in the Examples of WO 2015/092077 A1. The positions of the CDRs are shown in FIG. 1C, are explained in the Figure legend to FIG. 1 in WO 2015/092077 A1 and are underlined in present Table 2. In addition, or alternatively, the framework regions or complete V_{H} and/or V_{L} chain are 80% identical to the framework regions depicted in FIG. 1C or 1M of WO 2015/092077 A1, and shown in present Table 2, preferably 85%, 90%, 95%, 96%, 97%, 98%, 99% or 100% identical to the framework regions and V_{H} and/or V_{L} chain, respectively, depicted in FIG. 1C or 1M, WO 2015/092077 A1 and shown in present Table 2. Furthermore, cloning and expression of antibody NI-301.37F1 has been performed as described in WO 2015/092077 A1 in Examples 1 and 2 at pages 110 to 112 which methods are incorporated herein by reference.

Thus, in accordance with one embodiment of the method of the present invention, the anti-TTR antibody is characterized by the CDRs of the V_{H} and V_{L} chain and by the entire V_{H} and V_{L} chain, respectively depicted in FIGS. 1C and 1M of WO 2015/092077 A1 and shown in present Table 2. Thus, the antibody preferably comprises
(i) a variable heavy (VH) chain comprising the following VH complementary determining regions (CDRs) 1, 2, and 3, and/or a variable light (VL) chain comprising the following VL CDRs 1, 2, and 3:
   (a) VH-CDR1: positions 31-35 of SEQ ID NO: 19 (corresponds to SEQ ID NO: 10 of W (2015/092077 A1) or a variant thereof, wherein the variant comprises one or two amino acid substitutions,
   (b) VH-CDR2: positions 52-67 of SEQ ID NO: 19 (corresponds to SEQ ID NO: 10 of WO 2015/092077 A1) or a variant thereof, wherein the variant comprises one or two amino acid substitutions,
   (c) VH-CDR3: positions 100-109 of SEQ ID NO: 19 (corresponds to SEQ ID NO: 10 of WO 2015/092077 A1) or a variant thereof, wherein the variant comprises one or two amino acid substitutions,
   (d) VL-CDR1: positions 24-34 of SEQ ID NO: 21 (corresponds to SEQ ID NO: 12 of WO 2015/092077 A1) or a variant thereof, wherein the variant comprises one or two amino acid substitutions,
   (e) VL-CDR2: positions 50-56 of SEQ ID NO: 21 (corresponds to SEQ ID NO: 12 of WO 2015/092077 A1) or a variant thereof, wherein the variant comprises one or two amino acid substitutions, and
   (f) VL-CDR3: positions 89-97 of SEQ ID NO: 21 (corresponds to SEQ ID NO: 12 of WO 2015/092077 A1) or a variant thereof, wherein the variant comprises one or two amino acid substitutions; and/or
(ii) a VH chain and/or a VL chain, wherein
   (a) the VH chain comprises the amino acid sequence depicted in SEQ ID NO: 19 or SEQ ID NO: 23 (correspond to SEQ ID NO: 10 and SEQ ID NO: 53 of WO 2015/092077 A1), or a variant thereof, wherein the variant comprises one or more amino acid substitutions; and
   (b) the VL chain comprises the amino acid sequence depicted in SEQ ID NO: 21 (corresponds to SEQ ID NO: 12 of WO 2015/092077 A1), or a variant thereof, wherein the variant comprises one or more amino acid substitutions;
preferably wherein the VH and VL chain amino acid sequence is at least 90% identical to SEQ ID NO: 19 or 23 (corresponds to SEQ ID NO: 10 and SEQ ID NO: 53 of WO 2015/092077 A1), and SEQ ID NO: 21 (corresponds to SEQ ID NO: 12 of WO 2015/092077 A1), respectively.

In accordance with a preferred embodiment of the method of the present invention, the anti-TTR is NI-301.37F1 and comprises in its variable region or binding domain the amino acid sequences of the VH and VL chain of SEQ ID NO: 19 and SEQ ID NO: 21 or SEQ ID NO: 23 and SEQ ID NO: 21.

TABLE 2
Amino acid sequences and nucleotide sequences of the variable heavy (V_{H}) chain
and variable light (V_{L}) chain of antibody NI-301.37F1, wherein the CDRs in the
amino acid sequences are underlined. The regions in between the CDRs represent the
framework regions.

| | |
|---|---|
| Amino acid sequence of | QVQLQESGPGLVKPSETLSLTCSVSGGSIISRSSYWGWIRQPPGK |
| the V_{H} chain of antibody | GLEWIGGIYHSGNTYDNPSLKSRLTMSVDTSKNQFSLNLRSVT |
| NI-301.37F1 | AADTAVYYCARIVPGGDAFDIWGQGTMVTVSS |
| | SEQ ID NO.: 19 |
| Amino acid sequence of | DIQMTQSPSSLSASVGDRVTIACRASQSVGTYLNWYQQKRGKA |
| the V_{L} chain of antibody | PKLLIFAASSLQSGVPSRFSGSGSGTDFTLTISSLQPEDFATYYCQ |
| NI-301.37F1 | QSYSSPPTFGQGTKVEIK |
| | SEQ ID NO.: 21 |
| Amino acid sequence of | QLQLQESGPGLVKPSETLSLTCSVSGGSIISRSSYWGWIRQPPGK |
| the PIMC-V_{H} chain of | GLEWIGGIYHSGNTYDNPSLKSRLTMSVDTSKNQFSLNLRSVT |
| antibody NI-301.37F1 | AADTAVYYCARIVPGGDAFDIWGQGTMVTVSS |
| | SEQ ID NO.: 23 |
| Nucleotide sequence of | CAGGTGCAGCTGCAGGAGTCGGGCCCAGGACTGGTGAAGCC |
| the V_{H} chain of antibody | TTCGGAGACCCTGTCCCTCACCTGCAGTGTCTCTGGTGGCTC |
| NI-301.37F1 | CATCATCAGTAGGAGTTCCTACTGGGGCTGGATCCGCCAGCC |
| | CCCAGGGAAGGGGCTGGAGTGGATTGGGGGTATCTATCATA |
| | GTGGGAACACTTACGACAACCCGTCCCTCAAGAGTCGACTC |
| | ACCATGTCCGTAGACACGTCGAAGAACCAGTTCTCCCTGAAT |
| | CTGAGGTCTGTGACCGCCGCAGACACGGCTGTGTATTACTGT |
| | GCGAGGATAGTGCCGGGGGGTGATGCTTTTGATATCTGGGG |
| | CCAAGGGACAATGGTCACCGTCTCTTCG |
| | SEQ ID NO.: 18 |
| Nucleotide sequence of | GACATCCAGATGACCCAGTCTCCATCCTCCCTGTCTGCATCT |
| the V_{L} chain of antibody | GTAGGAGACAGAGTCACAATCGCTTGCCGGGCCAGTCAGAG |
| NI-301.37F1 | CGTTGGCACCTATTTAAATTGGTATCAGCAGAAAAGAGGGA |
| | AAGCCCCTAAACTCCTCATCTTTGCTGCATCCAGTTTGCAAA |
| | GTGGGGTCCCATCAAGGTTCAGTGGCAGTGGATCTGGGACA |
| | GATTTCACTCTCACCATCAGCAGTCTGCAACCTGAAGACTTT |
| | GCAACTTACTACTGTCAACAGAGTTACAGTTCTCCTCCAACG |
| | TTCGGCCAAGGGACCAAGGTGGAGATCAAA |
| | SEQ ID NO.: 20 |
| Nucleotide sequence of | CAGCTGCAGCTGCAGGAGTCGGGCCCAGGACTGGTGAAGCC |
| the PIMC-V_{H} chain of | TTCGGAGACCCTGTCCCTCACCTGCAGTGTCTCTGGTGGCTC |
| antibody NI-301.37F1 | CATCATCAGTAGGAGTTCCTACTGGGGCTGGATCCGCCAGCC |
| | CCCAGGGAAGGGGCTGGAGTGGATTGGGGGTATCTATCATA |
| | GTGGGAACACTTACGACAACCCGTCCCTCAAGAGTCGACTC |
| | ACCATGTCCGTAGACACGTCGAAGAACCAGTTCTCCCTGAAT |
| | CTGAGGTCTGTGACCGCCGCAGACACGGCTGTGTATTACTGT |
| | GCGAGGATAGTGCCGGGGGGTGATGCTTTTGATATCTGGGG |
| | CCAAGGGACAATGGTCACCGTCTCTTCG |
| | SEQ ID NO.: 22 |

Anti-Aβ antibodies may include Aducanumab (Sevigny et al. Nature 537 (2016), 50-56), Bapineuzumab (see review by Kerchner and Boxer, Expert Opin Biol Ther. 10 (2010), 1121-1130, DOI: 10.1517/14712598.2010.493872 including the primary literature cited therein), Gantenerumab (Bohrmann et al., Journal of Alzheimer's Disease 28 (2012), 49-69), Crenezumab (Guthrie et al., J Alzheimers Dis. 76 (2020), 967-979, DOI: 10.3233/JAD-200134), BAN2401 (Lannfelt et al., Alzheimers Res Ther 6 (2014), 16, DOI: 10.1186/alzrt246; WO 2007/108756 A1), Ponezumab (Burstein et al., Clin Neuropharmacol. 36 (2013), 8-13), and Solanezumab (Honing et al., N Engl J Med 378 (2018), 321-330, DOI: 10.1056/NEJMoa1705971).

Anti-Tau antibodies may include those described in Yanamandra et al., Ann Clin Transl Neurol 2 (2015), 278-288, DOI: 10.1002/acn3.176, WO 2012/049570 A1, and WO 2014/100600 A1), and in particular antibodies BIIB076 (6C5), BIIB092 (Gosuranemab), Bepranemab (UCB0107), C2N-8E12, and RG6100, which are also described in Medina, Int J Mol Sci. 19 (2018), 1160.

Anti-α-syn antibodies may include those described in WO 2012/177972 A1 and WO 2010/069603 A1, and in particular Prasinezumab (PRX002), Cinpanemab (BIIB054), ABBV-0805 and MEDI1341.

Anti-TDP-43, anti-SOD1, and anti-IAPP antibodies may include those described in WO 2013/061163 A2, WO 2012/080518 A1, in particular antibody NI-204.12G7, and WO 2014/041069 A1, in particular antibodies NI-203.26C11 and NI-203.11B12.

Anti-C9orf72 antibodies may include those described in WO 2016/050822 A2 and WO 2019/210054 A1, anti-LC antibodies may include antibodies 11-1F4 and NEOD00 (Muchtar and Gertz, Expert Opinion on Orphan Drugs 5 (2017), 655-663, and anti-PrP antibodies may include antibody PRN100.

Anti-SAA antibodies may include dezamizumab (GSK 2398852) and anti-HTT antibodies may include those as disclosed in WO 2016/016278 A2, in particular antibodies NI-302.35Cl and NI-302.31F11.

Further exemplary antibodies and equivalent binding molecules that bind to target antigens such as aggregated proteins as mentioned above are known in the art or can be identified using standard techniques. The assays of the invention enable rapid and accurate testing of such antibodies to confirm their ability to induce ADCP.

In this context, proteins prone to aggregation such as amyloidogenic proteins, in particular systemic amyloidogenic proteins, especially transthyretin (TTR), preferably being presented as protein aggregate, oligomer, fibril or proto-fibril, protein monomers, especially in misfolded conformation, as well as fragments thereof and synthetic peptides derived therefrom, which contain the epitope of the antibody or like Fc domain containing target antigen binding molecule, preferably a cyclic compound as defined above may be used as target antigen. In a preferred embodiment, the protein fragment or peptide contains an epitope of any one of the antibodies mentioned hereinbefore, most preferably an epitope of any one of the anti-TTR antibodies referred to hereinabove.

In a preferred embodiment of the present invention, anti-TTR antibodies are assessed for their potency to induce ADCP towards the aggregated protein TTR and towards the cyclic compound comprising an epitope of TTR as the preferred target antigens.

In one embodiment, the method of the present invention comprises at least the following steps:
i) spotting the target antigen such as aggregated protein or the cyclic compound to the wells of a microplate, i.e., microplates (96-well plates) were coated with the target antigen, preferably for 1 hour at 37° C. (protein aggregate) or over night at 4° C. (cyclic compound), preferably wherein the protein aggregate was diluted to a concentration of 10 μg/ml in PBS buffer pH 7.4, and wherein the cyclic compound was diluted to 3 μg/ml in PBS buffer pH 7.4;
ii) contacting the target antigen with the target antigen binding molecule under conditions allowing the formation of a binding molecule-target antigen complex, preferably for 30 min at 37° C.;
iii) contacting the complex comprising the binding molecule and the target antigen with effector cells, i.e., effector cells, also called reporter cells, were added to the complex, wherein the effector cells express an Fc receptor und an reporter gene under control of a response element that is responsive to activation by the Fc receptor, preferably wherein the effector cells are engineered cells, more preferably Jurkat cells expressing the FcγRI receptor and the luciferase gene under control of the NFAT transcription factor, and wherein the complex is incubated for 6 hours at 37° C.;
iv) adding a substrate solution, preferably a luminescence substrate solution; and
v) detecting the signal, preferably a luminescence signal with a luminometer.

Coating of the plate with the cyclic compound is preferably performed by immobilization on the plastic surface primarily by hydrophobic interaction but can also be performed by using the biotin-streptavidin system. However, as mentioned above, instead of spotting the target antigen to the wells of a microplate, the contacting of the target antigen with the target antigen binding molecule can also be performed in solution, without the target antigen being spotted to the wells of a solid support, such as a microplate.

In a preferred embodiment, a step of blocking non-specific binding sites is performed before step (ii), preferably wherein blocking was performed for 1 hour at room temperature with a blocking buffer containing 2% bovine serum albumin (BSA) and 0.1% tween-20 in PBS buffer. In one embodiment, the method of the present invention further comprises a step of preparing the target antigen before spotting it onto the microplate or the other solid support. As regards the protein aggregates, methods for preparing protein aggregates are well known in the art and may for example employ aggregation buffer as described in Example 3. The preparation of Ab fibrils is for example described in WO 2017/157961 A1. The preparation of the protein aggregate may further include the purification of the respective protein before subjecting to conditions allowing for aggregation. Purification can be performed via protein chromatography followed by a lectin column to eliminate residual immunoglobulins. Methods for preparing a cyclic compound are also known in the art as explained above. In a preferred embodiment, the peptide is cyclized via a disulfide bridge between cysteine residues in the linker; see above. The cyclic compound is prepared in solution and is not submitted to any specific procedure before use, and is thus in a native, monomeric form.

The method of the present invention may further comprise a step of controlling/verifying the quality of the protein aggregate or the cyclic compound. This can be performed by various methods for example by conventional ELISA and/or Biolayer interferometry (BLI) using an antibody known to bind the aggregated protein or the cyclic compound. Such methods are described in appended Examples 3 and 5.

Different assay set ups have been tested regarding flexibility in terms of plate layouts. In principle, dilution series of the target antigen binding molecule, e.g., antibody can be placed either in horizontal orientation (e.g., Well A1-A12) or vertical orientation (e.g., Well A1-H1). The number of dilution points can be freely selected (e.g., 8-, 12-, 16-point etc.), as well as the directionality (i.e., first well can either have lowest or highest ligand concentration). In single dose assays the position of references and positive controls can be freely selected by the user. However, in the course of experiments performed in accordance with the preset invention performing it surprisingly turned out that while both orientations work sufficiently well a vertical plate layout which allows three samples to be measured in parallel on the same plate and in triplicates provides the most reliable results. Accordingly, in one preferred embodiment the method of the present invention is performed using a vertical plate layout. Furthermore, in case of 96-well microplates the 24 outside wells presented a 24% larger variability than the one observed with the 60 inner wells and thus, preferably the inner wells are used when performing the method of the present invention.

In case an anti-TTR antibody is analyzed with the method of the present invention, the antibody NI-301.37F1 as characterized above can be used as control, either as quality control of the aggregated TTR batches or as positive control for the potency assay.

As can be derived from Examples 3,4 and 6, the method of the present invention has the capacity to detect changes in antibody activity and to detect potency loss of the antibody related to Fc domain alterations. The latter has been tested via subjecting the antibody to stress condition mimicking a loss in antibody potency. Accordingly, the method of the present invention has the capacity to detect changes in binding molecule activity by about at least±35% to ±50%.

The present invention further relates to a method of producing a pharmaceutical composition of the target antigen binding molecule as defined above, i.e., a binding molecule which comprises an Fc domain and which is preferably an antibody or any fragment or derivative thereof or an antibody mimetic.

In a first step, the binding molecule and the drug product, respectively, is provided, preferably produced. Means and methods for the recombinant production of antibodies, corresponding binding molecules, fragments, derivatives and mimics thereof are known in the art. In particular, their recombinant production in a host cell, purification, modification, formulation in a pharmaceutical composition and therapeutic use as well as terms and feature common in the art can be relied upon by the person skilled in art when carrying out the present invention as claimed (see, e.g., Antibodies A Laboratory Manual 2nd edition, 2014 by Cold Spring Harbor Laboratory Press, Cold Spring Harbor, New York, USA; Frenzel et al., Front Immunol. 4 (2013), 217, doi: 10.3389/fimmu.2013.00217; Lalonde and Durocher, Journal of Biotechnology 251 (2017), 128-140, DOI: 10.1016/j.jbiotec.2017.04.028; Tripathi and Shrivastava, Front. Bioeng. Biotechnol. 7 (2019), 420, DOI: 10.3389/fbioe.2019.00420), wherein also antibody purification and storage; engineering antibodies, including use of degenerate oligonucleotides, 5′-RACE, phage display, and mutagenesis, immunoblotting protocols and the latest screening and labeling techniques are described and. The production of DARPins is for example explained in Stumpp et al., Drug Discovery Today 13 (2008), 695-701 as well as in references cited therein and in Hanenberg et al., J Biol Chem 289 (2014), 27080-27089, DOI: 10.1074/jbc.M114.564013. Furthermore, the production of the drug product may be performed in any manner as desired and/or suitable for the drug product in question.

In a next step, the binding molecule is subjected to the method of the present invention. In particular, the binding molecule is subjected to the method for determining the potency of the binding molecule, in particular the potency to induce ADCP. The information derived from the assay is used as part of an assessment of whether the binding molecule may be used as a pharmaceutical composition or not, i.e., whether the drug product comprising the binding molecules fulfills the criteria to be injected into a patient as agreed with the regulatory authorities in a country, where an injection of the drug product may take place. Furthermore, the information is used to identify the binding molecule for use in the pharmaceutical composition.

In a further preferred embodiment of any of the afore-described embodiments of the method of the present invention, the target antigen binding molecule is formulated as a pharmaceutical composition with a pharmaceutically acceptable carrier, in particular that target antigen binding molecule which has been found useful by the method of the present invention. A useful binding molecule is for example such a binding molecule which shows an EC₅₀ value in the (sub)-nanomolar range when assessed with the method of the present invention, or which shows a similar potency as a reference standard, for example at least 80%, preferably at least 90%, preferably at least 95%, preferably at least 98%, preferably at least 99%, more preferably 100% in comparison to the potency of a positive control. Pharmaceutically acceptable carriers and administration routes can be taken from corresponding literature known to the person skilled in the art. The pharmaceutical compositions can be formulated according to methods well known in the art; see for example Remington: The Science and Practice of Pharmacy (2000) by the University of Sciences in Philadelphia, ISBN 0-683-306472, Vaccine Protocols 2^{nd} Edition by Robinson et al., Humana Press, Totowa, New Jersey, USA, 2003; Banga, Therapeutic Peptides and Proteins: Formulation, Processing, and Delivery Systems. 2^{nd} Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected in different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

The present invention also provides a process for preparing a pharmaceutical or diagnostic product comprising a target antigen binding molecule, wherein the potency of the binding molecule is at least 80%, preferably at least 90%, preferably at least 95%, preferably at least 98%, preferably at least 99%, more preferably 100% in comparison to the potency of a positive control to activate ADCP. The process comprises the production of the binding molecule as explained above, wherein a batch of said binding molecule is obtained. Afterwards, the potency of the batch is analyzed with the method of the present invention, in particular the potency of the batch to activate ADCP. The process further comprises the preparation of the pharmaceutical or diagnostic product from the batch, but only if the batch is determined to have a potency which is at least 80%, preferably at least 90%, preferably at least 95%, preferably at least 98%, preferably at least 99%, more preferably 100% in comparison to the potency of a positive control, in particular to activate ADCP.

The control is either a reference standard, an antibody which is known to have the potency to activate ADCP, for example an antibody which has been approved by the regulatory authorities, and/or the batch to be analyzed has been stored and/or was subjected to stress conditions and the control is the value of reporter gene activity of a sample taken from the batch or corresponding batch prior to storage and/or being subjected to said stress conditions.

The present invention also provides a method as described above, wherein said method is part of an application for marketing authorization for selling said drug product as a pharmaceutical composition. The present invention also provides a method for applying for marketing authorization for a drug product comprising the binding molecule, which method comprises describing the method of the present invention for determining the potency of the binding molecule of the drug product.

As mentioned above, the method of the present invention is used as a potency assay for batch release, i.e., the method of the present invention is useful for analyzing different batches from for instance the production of a given target antigen binding molecule.

Any continuing production of drug products will result in the production of different batches of product to be released as pharmaceuticals. A key feature in the production is to ensure that the different batches live up to the same standard. This standard is typically set in cooperation with the regulatory bodies. Typically, each batch will be tested and examined by a number of different assays to ensure that the batch is of sufficient quality to be approved for the market. This can be performed with the method of the present invention.

Thus, the present invention also relates to a method for analyzing and selecting at least one batch of a pharmaceutical composition of a target antigen binding molecule as defined above, wherein the method comprises in a first step the assessment of the potency of a sample of the batch, in particular its potency to activate ADCP, with the method of the present invention. As mentioned above, the reporter gene activity is a measure for the potency of the binding molecule and thus, the reporter gene activity of the sample is compared to the reporter gene activity of a control and the batch is selected, for which the sample shows greater, equal or no substantial less reporter gene activity compared to the control. In one embodiment, the batch is selected, for which the sample shows greater, equal or no less than 80%, preferably 90%, preferably 95%, preferably 98%, preferably 99%, more preferably 100% reporter gene activity compared to the control. The selected batch can be further packed, for example into a kit, and distributed to the costumer.

Thus, the present invention relates to a process for validating a batch of a target binding molecule, i.e., determining the quality of a target antigen (e.g., aggregated protein) binding molecule, for distribution, wherein a sample of the batch is tested for its potency to activate ADCP with the method of the present invention and wherein the batch is validated for distribution only if the potency of the sample of the batch to activate ADCP is at least 80%, preferably at least 90%, preferably at least 95%, preferably at least 98%, preferably at least 99%, more preferably 100% in comparison to the potency of a positive control to activate ADCP.

In a preferred embodiment, the methods are particularly useful for analyzing and selecting a batch of a pharmaceutical composition comprising an anti-TTR antibody and for validating a batch of an anti-TTR antibody for distribution, respectively. The control can be a reference standard and/or in case the batch to be analyzed has been stored and/or was subjected to stress conditions, the control can be the value of reporter gene activity of a sample taken from the batch or corresponding batch prior to storage and/or being subjected to said stress conditions.

The binding of the binding molecule of the drug product to the Fc receptor is compared to the binding of the reference standard to the Fc receptor, and the therapeutic efficacy of the binding molecule of the drug product is assessed from its ability to bind the Fc receptor to the same or substantially the same degree as the reference standard.

As mentioned above, the potency of the sample of the batch should be preferably at least 80%, preferably at least 90%, preferably at least 95%, preferably at least 98%, preferably at least 99%, more preferably 100% in comparison to the potency of the reference standard. However, the particular degree to which the FcR binding profile of the binding molecule of the drug product and the FcR binding profile of the reference standard may differ may be established on a case-to-case basis and may for instance be determined in cooperation with the appropriate regulatory body.

To be able to determine FcR binding in a reliable and consistent manner, the FcR binding of the binding molecule of the drug product and the reference standard should be performed using the same assay, preferably using the assay of the present invention. The determination of the binding of the reference standard will typically be performed first to establish a standard that any following batches of the binding molecule can be compared with. However, the determination of the binding of the reference standard may also be performed at the same time or after the determination of the FcR binding of the binding molecule of the drug product.

The present invention further relates to the use of the target antigen binding molecule as defined above, the cyclic compound as defined above, and/or the effector cell as defined above in the method according to the present invention. In particular, the effector cell is engineered to express a human Fc receptor FcγRI (CD64) and harbors a reporter gene under the control of a response element that is responsive to activation by the Fc receptor.

Furthermore, the present invention relates to a kit, which comprises at least
(i) a population of effector cells genetically engineered to express an Fc receptor and harboring a gene encoding a reporter under control of a response element that is responsive to activation by the Fc receptor;
(ii) a corresponding substrate for the reporter; and optionally
(iii) the target antigen;
(iv) a microtiter plate, preferably a 96- or 384-well plate including a lid;
(v) recommendations for buffers, diluents, substrates and/or solutions as well as instructions for use;
(vi) washing, blocking and assay/sample dilution buffer; and/or
(vii) a positive control target antigen binding molecule, preferably an antibody.

Preferably, this kit is adapted to carry out the method of the present invention, in particular to assay the potency of a binding molecule comprising an Fc domain to induce ADCP. Thus, the instructions for use concern in a preferred embodiment instructions for use of the kit in a method of determining the potency of a target antigen binding molecule comprising an Fc domain, and preferably instruction for use how to perform the assay of the present invention.

In a preferred embodiment, the population of effector cells is a population of Jurkat cells expressing FcγR, preferably FcγRI and a gene encoding a luminescence protein, preferably a luciferase under control of the NFAT transcription factor and wherein the kit comprises a luminescence substrate solution. Furthermore, the target antigen is preferably an aggregated protein, more preferably aggregated TTR, or a cyclic compound comprising the epitope of a target binding molecule, more preferably the epitope of an anti-TTR antibody and an epitope of TTR, respectively, and the binding molecule is an anti-TTR antibody.

The method does not necessarily need to be performed on a microtiter plate, but any solid support to which the target antigen can be spotted or any vial in which the assay components can be incubated in would be suitable.

The present invention further relates to a composition comprising the target antigen binding molecule of the present invention which has been analyzed, validated and selected according to the present invention, wherein the composition further comprises a pharmaceutically acceptable carrier.

In order to verify that the analyzed binding molecule indeed triggers phagocytosis leading to engulfment of the target antigen, for instance the protein aggregate or the cyclic compound, in vitro phagocytosis assays have been performed as described in Examples 1 and 2. These assays show that antibody NI-301.37F1_W1 indeed triggers phagocytosis of the TTR aggregate. Thus, the method of the present invention for assaying the potency of the binding molecule may be combined with an in vitro phagocytosis assay.

Furthermore, the binding of the analyzed binding molecule to its corresponding antigen might be verified by methods known in the art, for example via ELISA or BLI as shown in Examples 3 and 5. Thus, the method of the present invention for assaying the potency of the binding molecule may be combined with a method for determining the binding of the binding molecule to its antigen.

In a particular preferred embodiment of any one of the methods and kits of the present invention described hereinbefore and/or characterized in the claims, (1) the potency, i.e., effector function to be determined is antibody-dependent cell phagocytosis (ADCP), (2) the target antigen is an amyloidogenic protein aggregate involved in systemic amyloidosis, most preferably TTR, or a protein fragment or peptide derived thereof, preferably in cyclic form, which comprises an epitope of an amyloidogenic protein, most preferably TTR, (3) the Fc receptor is a human Fc receptor FcγRI (CD64), the effector cell is a Jurkat cell, preferably wherein the cell does not overexpress FcγRIIa (CD32a) and FcγRIII (CD16), the response element is an NFAT (Nuclear Factor of Activated T cells) response element, the reporter gene encodes a luciferase and the target binding molecule is an IgG1 antibody, such as an IgG1, λ antibody or an IgG1, K antibody.

As mentioned in the “Summary of the invention” and described hereinbefore, in a further aspect the present invention relates to a cyclic compound which comprises a peptide comprising an epitope from an amyloidogenic protein involved in systemic amyloidosis.

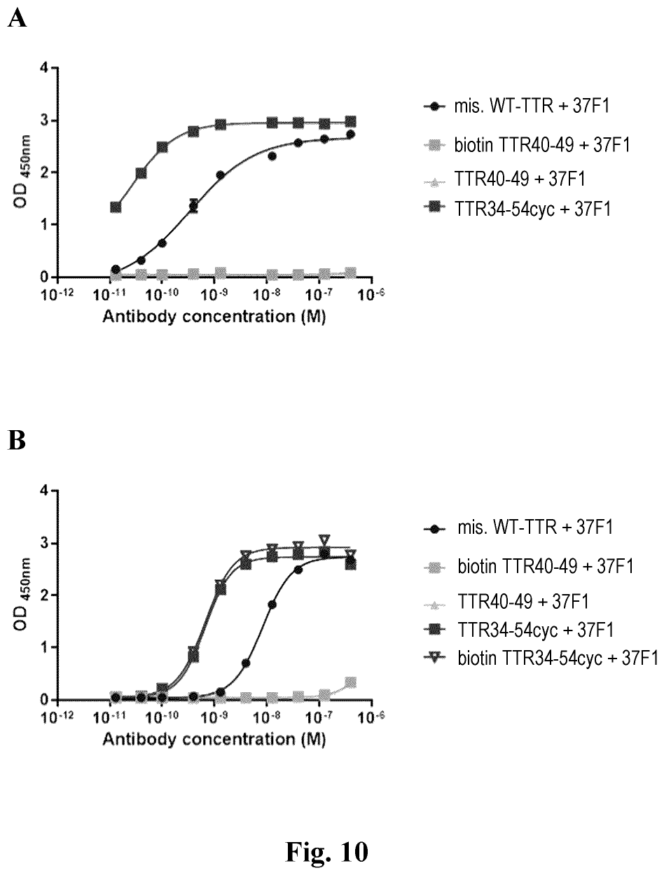

In particular, during the course of the experiments performed in accordance with the present invention, it was surprisingly found that a cyclic peptide comprising an epitope of TTR, in particular the epitope recognized by the anti-TTR antibody NI-301.37F1 (WEPFA (SEQ ID NO: 1), which binds selectively with high affinity to TTR aggregates of either wild-type or variant TTR as described in WO 2015/092077 A1, is an excellent target antigen in ELISA and ADCP assays. As described in Example 5 and illustrated in FIG. 10, the antibody displays highly specific binding to said cyclic compound, wherein the binding affinity of the antibody to the cyclic peptide is even an order of magnitude higher than to its native target antigen, i.e., misfolded TTR against which the antibody had been originally screened and identified. This remarkable effect was unexpected and advantageous since such cyclic could not only substitute full-length amyloidogenic proteins and the preparation of aggregates/fibrils thereof, which is prone to variability and more time consuming than preparation of a cyclic peptide, but, as illustrated in Examples 5 and 6, and shown in FIGS. 10 to 12, the cyclic compound represents an excellent target antigen in binding assays like ELISA and functional assays such ADCP which require high sensitivity and reproducibility.

Originally, the cyclic TTR peptide was designed to resolve crystal structures of a Fab fragment of antibody NI-301.37F1 in complex with its TTR antigen to gain information about the three-dimensional structure of the antibody-antigen complex to understand its mechanism of action. It was a coincidence that the cyclic TTR peptide was used to replace full-length recombinant TTR protein in an ELISA assay for the determination of antibody NI-301.37F1, i.e., the IgG antibody, and surprisingly revealed that the ELISA assay became much more sensitive and reliable compared to the use of recombinant TTR protein; see Example 5 and FIG. 10. Subsequent experiments even more surprisingly demonstrate that use of the cyclic TTR peptide substantially improves the sensitivity and reliability of potency assay of the present invention.

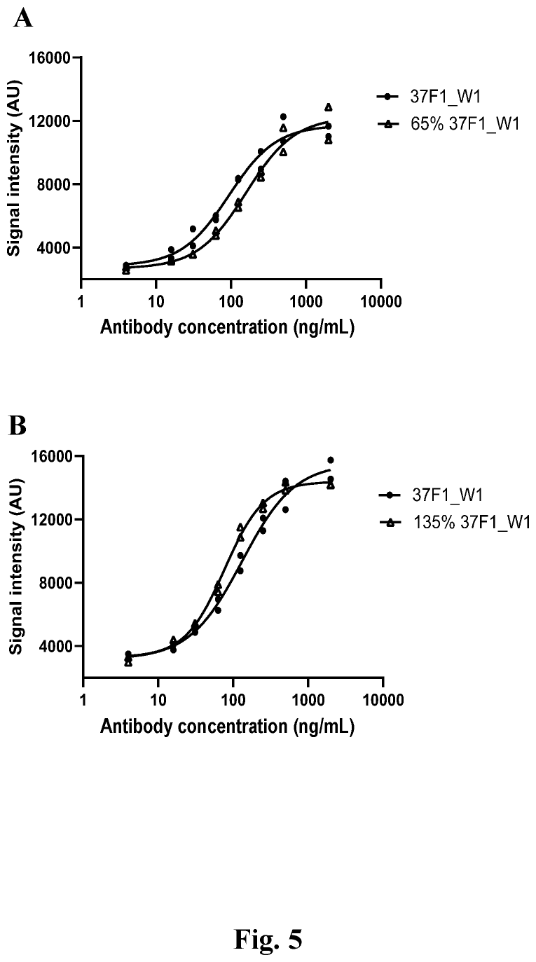

In the following, analysis of cryo electron microscopy (cryo-EM) structures revealed that the two ends of the unresolved loop at in contact with each other and suggests that on this basis the epitope and peptide sequence for the cyclic peptide and cyclic compound, respectively, could have been selected as well. Accordingly, the use of cryo-EM structures, in addition or alternatively to peptide design in Fab-peptide antigen crystallographic structures may be used to choose the appropriate epitope and amino acid sequence containing the same for the design of the cyclic peptide of the present invention, which displays as advantageous properties as the cyclic TTTR peptide that has been experimentally proven to be so effective and a reliable tool in ELISA and ADCP assay. As mentioned hereinbefore, it is noteworthy that it has been shown by cryo-EM studies that the amyloid structures of systemic amyloidogenic proteins such as ATTR and AL amyloidosis caused by misfolding of immunoglobulin light chains (LCs) are on the one hand similar, but on the other hand substantially differ from those of local amyloidogenic proteins such as tau; see FIG. 5 in Schmidt et al., Nat. Commun. 10 (2019), 5008, https://doi.org/10.1038/s41467-019-13038. Therefore, it is prudent to expect that the present results for cyclic peptides derived from TTR can also be applied to at least other systemic amyloidogenic proteins.

Methods for generating crystal structures of an antibody and its Fab fragment, respectively, to a peptide and its complex with peptide antigen are well known to the person skilled in the art; see, e.g., Amit et al., Science 233 (1986), 747-753. The same applies to cryo electron microscopy; see, e.g., Schmidt et al. (2019), supra.

This finding now opens the opportunity to generate further cyclic compounds comprising epitopes of other amyloidogenic proteins. For example, once an epitope has been selected and fragment or peptide sequence of the amyloidogenic protein has been selected, the program PEP-FOLD, which can predict peptide structures from amino acid sequences and when applied to the TTR cyclic peptide seems to reasonably predict the presentation of the epitope, could be used to design further cyclic peptides, such as those described herein, that mimic for example the antibody binding epitopes of amyloidogenic proteins.

Accordingly, the findings made in the experiments performed within the scope of the present invention allow the generation of cyclic compounds of epitopes from any kind of amyloidogenic proteins, such as those described herein, in particular when derived from a systemic amyloidogenic protein.

Accordingly, in further aspect, the present invention relates to a cyclic compound as described hereinbefore and linear precursor thereof comprising a peptide containing an epitope of a systemic amyloidogenic protein, the epitope preferably being accessible to binding by an antibody only in the misfolded and/or aggregated form of the protein, as in the case of a neoepitope, and/or the epitope being at least not present in the physiologically active form of the protein, e.g. in the case of an epitope accessible in the monomer of the TTR protein, which is hidden in the physiologically active tetramer and is no longer accessible to antibody binding. As illustrated in Example 6, the cyclic compound of the present invention is particularly useful in the potency assay of the present invention.

Most preferably, the cyclic compound of the present invention comprises the amino acid sequence WEPFA (SEQ ID NO: 1).

In one embodiment, the cyclic compound and precursor thereof, respectively, or the protein fragment or peptide within the cyclic compound of the present invention is further derivatized or modified. For example, proteins and/or other agents may be coupled to the cyclic compound, which may for example act as a probe in in vitro studies. For this purpose, any functionalizable moiety capable of reacting (e.g., making a covalent or non-covalent but strong bond) may be used. Those proteins and/or other agents can be for example a carrier protein such as bovine serum albumin (BSA) used for immunoblots or immunohistochemical assays.

The present invention further relates to a composition comprising the cyclic compound of the present invention or a linear precursor thereof. The composition can have further excipients, such as buffers, stabilizing agents, and/or diluents.

As indicated in Example 5, the antigen binding molecule, here the anti-TTR antibody, showed a strong binding affinity to the cyclic peptide in ELISA assays. Accordingly, the cyclic compound is a suitable target antigen in assays that are used to detect and quantify antigen binding molecules, such as antibodies.

The present invention thus further relates to the use of the cyclic compound of the present invention or of the composition of the present invention in any kind of assay which concerns the analysis of the interaction between a target antigen binding molecule and a target antigen, for example the detection, which can also include the quantification of a target antigen binding molecule. In a preferred embodiment, such an assay is an ELISA assay. In a further preferred embodiment, the present invention relates to the use of the cyclic compound of the present invention or of the composition of the present invention for determining the potency of an antigen binding molecular, such as an antibody or any other binding molecule comprising an Fc domain, preferably of an antibody as defined hereinbefore. The determination of the potency is preferably performed with the assay of the present invention.

Furthermore, the cyclic compound of the present invention or a composition comprising the same can be used for the detection of autoantibodies against amyloidogenic proteins or fragments, oligomers or aggregates thereof. The cyclic compound of the present invention is in particular suitable for the detection of autoantibodies against TTR and identify antibodies equivalent to, for example NI-301.37F1. Likewise, the cyclic peptide of the present invention can be used for screening of antibodies against amyloidogenic proteins and in particular of anti-TTR antibodies in general for example by phage display.

In addition, the cyclic peptide of the present invention can be used for studying the pharmacokinetic profile, i.e., the half-life of the antibody in the plasma of in vivo non-human animal trials as well as clinical trials in humans, for example with antibody NI-307.37F11 (NI006), or NNC6019-0001 (PRX004). Furthermore, the cyclic compound can be used during the course of for example antibody treatment to measure the plasma concentration of the antibody and support the dosing for keeping a sustained level of the antibody. The cyclic peptide can also be used to identify antibodies equivalent to known antibodies and in particular, equivalent to the mentioned anti-TTR antibodies, in particular antibody NI-307.37F11, for example by competition assays which are commonly known in the art. Thus, all the uses are also part of the present invention.

Furthermore, the present invention concerns a kit comprising at least the cyclic compound of the present invention or a linear precursor thereof, optionally with reagents and instructions for use. The kit is preferably useful for detecting the interaction between a target antigen binding molecule and a target antigen, for example the detection, which can also include the quantification of a target antigen binding molecule, and most preferably for determining the potency of an antigen binding molecule which comprises an Fc domain, such as an antibody. In a preferred embodiment, determination of the potency is preferably performed with the assay of the present invention. In a further preferred embodiment, the antigen binding molecule is an antigen binding molecule as defined hereinbefore, preferably an antigen binding molecule comprising an Fc domain, such as an antibody, and most preferably an anti-TTR antibody. Accordingly, the kit can be used for the purposes listed above.

In one embodiment, the kit of the present invention comprising the cyclic compound further comprises
(i) a population of effector cells engineered to express a human Fc receptor FcγR and harbor a reporter gene under the control of a response element that is responsive to activation by the Fc receptor
(ii) a corresponding substrate for the reporter; and optionally
(iii) a solid support, preferably a microtiter plate, preferably a 96-well plate including a lid;
(iv) washing, blocking and assay/sample dilution buffer, and/or
(v) a monomer control of the target antigen and/or a positive control anti-target antigen antibody.

In a preferred embodiment, the population of effector cells is a population of Jurkat cells expressing FcγR, preferably FcγRI and a gene encoding a luminescence protein, preferably a luciferase under control of the NFAT transcription factor and wherein the kit comprises a luminescence substrate solution.

In addition, the cyclic compound of the present invention, and the linear precursor thereof, are especially useful in methods for identifying and optionally obtaining an antibody and equivalent binding molecules such as of the type described hereinbefore, which binds to an amyloidogenic protein involved in systemic amyloidosis, the method typically comprising the steps of:
(a) providing, optionally producing one or more potentially amyloidogenic protein binding antibodies or a source thereof;
(b) subjecting the one or more of potentially amyloidogenic protein binding antibodies or source thereof to a binding assay comprising the cyclic compound of the present invention; and
(c) identifying and optionally obtaining an antibody (subject antibody) that has been determined to bind to the cyclic compound.

This method can be combined with the potency assay of the present invention, and/or any other suitable method for further determining the diagnostic or preferably therapeutic utility of the subject antibody. As mentioned, the subject antibody may also be a different kind of antigen binding molecule.

Hence, a further embodiment of the present invention consists in a method of producing a pharmaceutical composition comprising an antibody which binds to a systemic amyloidogenic protein, the method comprising at least the steps of:
(a) providing, optionally producing one or more potentially amyloidogenic protein binding antibodies or a source thereof;
(b) subjecting said one or more potentially amyloidogenic protein binding antibodies or a source thereof to a binding assay comprising the cyclic compound of the present invention;
(c) identifying and optionally obtaining an antibody (subject antibody) that binds to the cyclic compound; and
(d) formulating the antibody identified and optionally obtained in step (c) or a derivative thereof with a pharmaceutically acceptable carrier.

The source of antibodies is not limited and comprises natural as well as synthetic antibodies obtained, for example from immunized laboratory animal such as a rodent, preferably mouse, most preferably Ig humanized mouse; human blood or a fraction thereof preferably comprising memory B cells; recombinant antibody libraries such as phage, yeast, and ribosome systems or mammalian cell systems such as CHO and HEK; see also the “Detailed description of the invention” for further sources of antibodies and other target binding molecules. In one embodiment, nanobodies, also known as VHHs, which originated from the serum of Camelidae may be screened with the cyclic compound of the present invention; see, e.g., Lyu et al., Anal. Chem. 94 (2022), 7970-7980; Muyldermans, The FEBS Journal 288 (2021) 2084-210. In this context, an IgG antibody of binding fragment thereof known to bind the amyloidogenic protein may be used a as reference antibody or source for identification and preparation of the nanobody, respectively. Likewise, synthetic alternatives to antibodies which may be designed computational modeling can be screened, for example modular peptide binders such as designed armadillo repeat proteins (dArmRPs); see, e.g., Gisdon et al., Biological Chemistry 403 (2022), 535-543.

The binding assay used in the methods mentioned above preferably comprise ELISA such as performed in Examples 5 and 7.

In a preferred embodiment of the methods of the present invention for identification and obtaining subject antibodies and their further use in being formulated in a pharmaceutical composition and drug development, respectively, the antibody identified and optionally obtained in step (c) competes with a reference antibody for binding the amyloidogenic protein, preferably wherein the subject antibody has a lower ECso for the amyloidogenic protein than the reference antibody. Preparation and formulation of the subject antibody and like target binding molecule obtained by the method of the present invention can be performed as described for the target antigen binding molecule, supra.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific example which are provided herein for purposes of illustration only and is not intended to limit the scope of the invention.

### EXAMPLES

### Example 1: In Vitro Phagocytosis Assay Using Human-Derived Macrophages

Phagocytosis of misfolded TTR triggered by antibody NI-301.37F1_3 was determined in an in vitro assay including human-derived macrophages, fluorescently labeled L55P-TTR protein, and the ATTR selective NI-301.37F1 3 antibody.

Human-derived macrophages were differentiated in vitro from fresh human monocytes. In brief, a fresh blood donation was received, PBMCs were prepared and the monocytes were extracted by negative depletion on a magnetic column (Miltenyi, Monocyte isolation kit II). Monocytes were then differentiated into M2 macrophages by cultivating them for a minimum of 10 days in macrophage-serum free medium (M-SFM, Life technologies) supplemented with 100 ng/ml macrophage colony-stimulating factor (M-CSF, Miltenyi). Between 10 and 15 days after differentiation initiation, macrophages were detached with trypsin and distributed into 96-or 24-well plates at a density of 500,000 cells/ml. The phagocytosis experiment was performed on the following day

The L55P-TTR mutant (Wako, Osaka, Japan) was selected for the in vitro phagocytosis experiment because this mutation strongly destabilizes TTR tetramer and leads to the generation of misfolded TTR proteins under physiological conditions. The L55P-TTR protein was coupled with a fluorescent dye to allow direct detection of TTR in macrophages (Atto 488 Protein labeling kit from Sigma, or pHrodo Green labeling kit from ThermoFischer).

The antibodies NI-301.37F1_3 and isotype control were coupled with a fluorescent dye (Atto 550 Protein labeling kit from Sigma, or pHrodo Red labeling kit from ThermoFischer) following standard procedure to allow direct detection in macrophages.

For the phagocytosis assay, macrophages were pre-incubated for 30 min with fucoidan (Sigma) to prevent unspecific phagocytosis mediated by scavenger receptors, and Fc-receptor inhibitor (Miltenyi) as negative control condition. L.55P-TTR-488 and NI-301.37F1_3-550 or isotype-550 were co-incubated for at least 15 min at room temperature before addition to the macrophages. Phagocytosis was performed in triplicates, with incubation for 2 hours at 37° C. in presence of fucoidan at 0.5 mg/ml, L55P-TTR-488 at 7 μg/ml, NI-301.37F1 3-550 or isotype-550 antibodies at concentrations from 0 to 80 nM, and FcR block at 1:100 dilution. The reaction was stopped by washing cells twice with PBS and keeping the plate on ice until measurement. For FACS analysis, macrophages were washed with PBS/EDTA, trypsinized, detached and stored on ice until quantification.

A standard fluorescence plate reader was used to quantify the total level of L55P-TTR-488 incorporated by macrophages. Similar experiments were quantified by FACS, to count the number of macrophages having incorporated both L55P-TTR and NI-301.37F1 3. The experiment was also repeated with macrophages coated on coverslips, which, after washing, fixation, and mounting, were used for confocal microscopy.

Antibody-mediated TTR uptake was concentration-dependent and required low antibody concentration (FIG. 1A). The uptake was strongly increased by NI-301.37F1_3 already at 1 nM concentration and reached saturation at 10 nM under the assay conditions used. Phagocytosis was mediated by Fc receptors as indicated by the complete inhibition in presence of 1% FCR block and required specific antibody-target interaction as indicated by the absence of TTR uptake in presence of isotype control antibody. A parallel experiment was analyzed by FACS to quantify specifically cells that are positive for both TTR and NI-301.37F1_3. The frequency of double-positive cells increased from a background level of 3% to 6% in presence of 10 nM NI-301.37F1_3, and increased further to 16% in presence of 80 nM NI-301.37F1_3 (FIG. 1B).

To further refine the analysis, the experiment was repeated using L55P-TTR and NI-301.37F1_3 proteins labelled with the pH sensitive fluorescent dyes pHrodo Green and pHrodo Red, respectively. These dyes present a strong increase in fluorescence at acidic pH, allowing to specify the analysis to those cells which have internalized TTR and NI-301.37F1_3 in phagolysosomal vesicles. In agreement with previous experiments, the frequency of double-positive cells increased from a basal level of 3.5% to 5.5% in presence of 10 nM NI-301.37F1_3, and increased further to 8.8% in presence of 80 nM NI-301.37F1_3 (FIG. 1C). This antibody-dependent phagocytosis of TTR was triggered specifically by NI-301.37F1_3 and not by the isotype control antibody, which did not induce phagocytosis above background level at 10 and 80 nM. Examination of macrophages by confocal microscopy supported these results, with active macrophages presenting a large number of vesicles positive for both TTR and NI-301.37F1_3. In summary, the results indicate that NI-301.37F1_3 induces in a concentration-dependent manner the phagocytosis of L55P-TTR by human macrophages. Phagocytosis was mediated by Fc receptors and required specific interaction of the antibody with its target protein. Upon phagocytosis, the antibody-target complexes were targeted to acidic compartments, most likely the phagolysosomal system for target degradation.

### Example 2: In Vitro Phagocytosis Assay Using THP1 Cells

The phagocytosis assay shown in Example 1 in principle showed that NI-301.37F1 3 has the capacity to activate ATTR phagocytosis, but this approach suffered from the variability in phagocytic activity between macrophages obtained from different blood donors. To eliminate this source of variability, the in vitro phagocytosis assay was redeveloped using the human monocytic THP1 cell line instead of fresh PBMCs. Once established, the ATTR phagocytosis assay using THP1 cells was evaluated for its capacity to detect a loss in antibody potency mimicked by a 30% reduction in antibody concentration.

THP1 cells (Sigma; 88081201) were cultivated in spinner flasks, using cell culture medium RPMI 1640 (ATCC, Manassas, Virginia, USA; ATCC1640 30-2001) supplemented with 20% fetal bovine serum (FBS), 1× Penicillin/Streptavidin and 0.05 mM 2-mercaptoethanol during cell growth. Cells were kept at a density of between 10⁵ to 10⁶ cells/ml for an optimal dividing rate. For the phagocytosis assay, THP1 cells were distributed in 96-well plates at a density of 200,000 to 400,000 cells/ml and differentiated using phorbol 12-myristate 13-acetate (PMA; Sigma) at 25 ng/ml for 48 hours, followed by PMA plus human interferon gamma (IFNY; Sigma) at 20 ng/ml for another 48 hours.

The monomeric F87M/L110M-TTR mutant (AlexoTech AB, Umea, Sweden; T-509-10) was selected because this double mutation prevents formation of TTR dimers and tetramers, therefore facilitates protein labeling and formation of ATTR aggregates. The F87M/L110M-TTR protein was coupled with Atto-488 fluorescent dye following kit instructions (Sigma, 38371), then aggregated at 1 mg/ml in aggregation buffer (50 mM acetate-HCl, 100 mM KCl, 1 mM EDTA, pH 3.0) for 4 hours at 37° C., resulting in the production of fluorescently labeled misfolded TTR aggregates (mis.TTR-488).

For the phagocytosis assay, antibody dilution series were prepared in Life Cell Imaging Solution (L.CIS, ThermoFischer A14291DJ) supplemented with fluorescently labelled misfolded TTR at 150 μg/ml, and preincubated for 2 hours at RT. In parallel, cell culture medium was replaced with LCIS supplemented with fucoidan at a final concentration of 0.1 mg/ml, and preincubated for 30 min at 37° C. The phagocytosis assay was started by adding 100 μL of mis. TTR-488/antibody solution to THP1 cells, followed by 90 min incubation at 37° C. The assay was stopped by washing cells with ice-cold PBS, and the wells filled with LCIS supplemented with background suppressor in the final wash step. Intracellular mis. TTR-488 fluorescence was measured using a plate reader with excitation set at 498±5 nm, emission 520±5 nm, 100 ms duration, bottom reading, using all reading sites per well. The phagocytosis assay was conducted using NI-301.37F1_3 at concentrations ranging from 0.02 to 5 nM as reference (1x_NI301A), and a similar dilution series prepared with NI-301.37F1_3 at 0.7× the reference concentration (0.7x_NI-301.37F1_3). This second condition was used to evaluate if the assay had the capacity to detect a potential loss of antibody activity, which was mimicked in this experiment by a 30% reduction in NI-301.37F1_3 concentration.

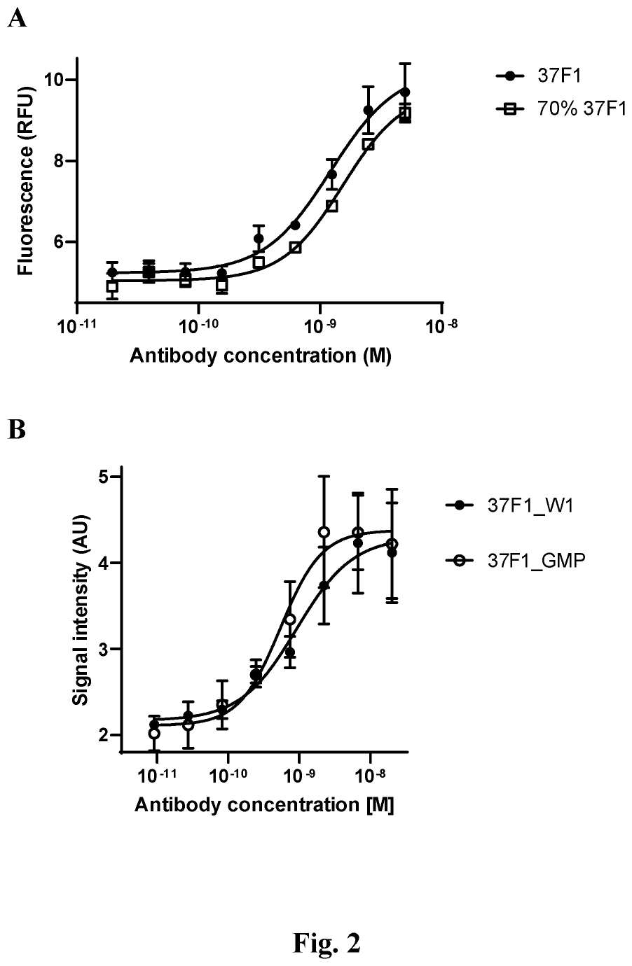

The results indicated that both 1x and 0.7x_NI-301.37F1_3 triggered phagocytosis of mis. TTR-488 by THP1 cells in a concentration-dependent manner. 1x_NI-301.37F1_3 dose-response was characterized by an ECso of 1.2 nM, and 0.7x_NI-301.37F1_3 dose-response by an ECso of 1.5 nM (FIG. 2A, average LSD of triplicates). The 25% lower potency observed with 0.7x_NI-301.37F1_3 was in good agreement with the 30% lower antibody concentration in this sample. Average+SD of triplicates.

The in vitro phagocytosis assay was further evaluated using NI-301.37F1_W1 non-GMP drug product and NI-301.37F1_W1 GMP drug substance. NI-301.37F1_3 and NI-301.37F1_W1 antibodies have the same sequence and differ only in their production and purification methods.

Both compounds were prepared as dilution series ranging from 0.09 to 20 nM. The results indicated that both NI-301.37F1 W1 non-GMP DP and NI-301.37F1_W1 GMP DS triggered phagocytosis of mis. TTR-488 by THP1 cells in a concentration dependent manner. NI-301.37F1 W1 non-GMP DP dose-response was characterized by an EC₅₀ of 0.92 nM, and NI-301.37F1_W1 GMP DS dose-response by an EC₅₀ of 0.54 nM (FIG. 2B). In this assay, however, triplicates presented a large variability which precluded calculation of the confidence intervals for the EC₅₀s.

### Example 3: ADCP Assay Using FcγRI Reporter Cell Line for Measuring the Potency of an Antigen Binding Molecule to Activate Phagocytosis of a Target Protein

An ADCP assay for determining the potency of a protein aggregate binding molecule was developed. The assay uses a reporter cell line expressing the human Fcγ receptor 1 (FcγR1) and has exemplary been evaluated for its capacity to measure the potency of antibody NI-301.37F1_W1 to activate phagocytosis of misfolded wildtype TTR (mis. WT-TTR) in vitro. Antibody NI-301.37F1_W1 and NI-301.37F1_3 both refer to antibody NI-301.37F1 described in international application WO 2015/092077 A1 and only differ in their recombinant production and method of purification.

Preparation and Characterization of Mis. WT-TTR

Wild-type TTR protein purified from human plasma was obtained from Bio-Rad Laboratories, Inc. (California, USA; 7600-0604) and submitted to a custom purification through protein A/G chromatography followed by a lectin column to eliminate residual immunoglobulins. Plasma-purified WT-TTR was provided as a solution at a concentration of 1 mg/ml in PBS buffer. Misfolded WT-TTR aggregates (mis. WT-TTR) were prepared in vitro by diluting WT-TTR stock solutions to a concentration of 200 μg/ml in aggregation buffer (50 mM acetate-HICI, 100 mM KCl, 1 mM EDTA, pH 3.0) followed by incubation for 4 hours at 37° C. with shaking at 1000 rpm. mis. WT-TTR was aliquoted and stored until use at −20° C. The quality of mis. WT-TTR was confirmed by ELISA and Biolayer interferometry (BLI).

For ELISA, 96-well microplates were coated for 1 hour at 37° C. with mis. WT-TTR diluted to a concentration of 10 μg/ml in PBS buffer pH7.4. Non-specific binding sites were blocked for 1 hour at room temperature (RT) with a blocking buffer containing 2% bovine serum albumin (BSA) and 0.1% tween-20 in PBS buffer. NI-301.37F1_3 antibody (Neurimmune AG, Zurich, Switzerland; NI-301.37F1) was diluted in duplicates to the indicated concentrations in PBS and incubated overnight at 4° C. Binding was determined using an anti-human IgG antibody conjugated with horseradish peroxidase (HRP), followed by measurement of HRP activity in a standard colorimetric assay (ThermoFisher Scientific Inc., Waltham, Massachusetts, USA). Data were analyzed with the Prism software from GraphPad. EC50 values were estimated using non-linear regression of individual data points using log (agonist) versus response model with variable slope. Data fitting was performed with the least square regression method.

BLI was performed on an Octet RED96 machine (Molecular Devices, LLC, San José, California, USA) equipped with anti-human capture sensors. Binding kinetics were measured at 25° C. in 1x Kinetic buffer (assay buffer). NI-301.37F1_3 or NI-301.37F1_W1 antibodies were diluted at 5 g/ml in assay buffer and loaded on sensors for 300 s. Mis. WT-TTR aggregates were diluted in assay buffer at 6 different concentrations, and a buffer-only condition was run in parallel on the 7th and 8th sensors, the latter being used as reference. Association and dissociation were measured for 600 s each. Data were analyzed in Data Analysis 8.2 using reference subtraction (buffer-only condition). A simple 1:1 binding model was used for kinetic analysis.

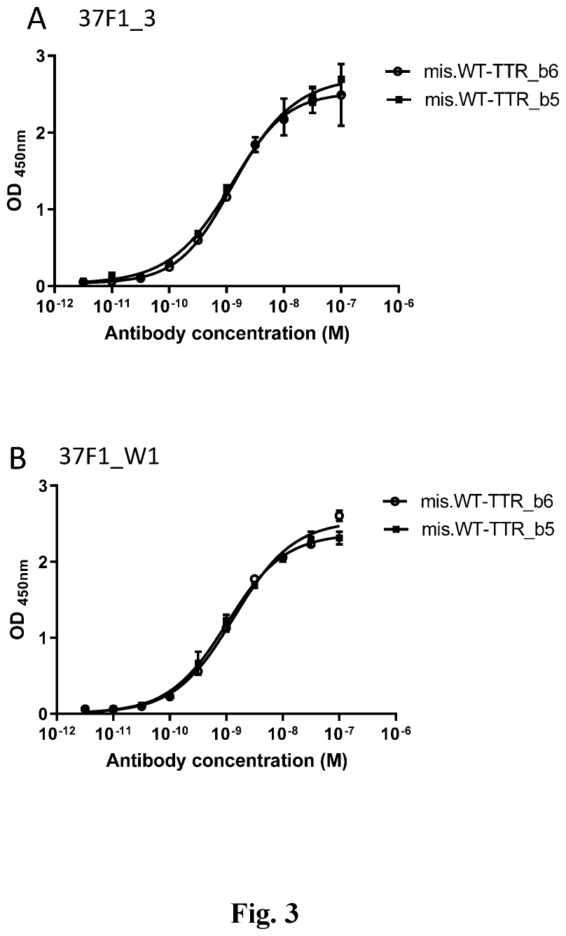

Mis. WT-TTR batch 6 (mis.WT-TTR_b6) was quality-controlled by comparing it to the previous batch of mis. WT-TTR (mis. WT-TTR_b5). The analysis was conducted by measuring NI-301.37F1_3 and NI-301.37F1_W1 binding using ELISA and BLI. The ELISA results showed that NI-301.37F1_3 (FIG. 3A) and NI-301.37F1_W1 (FIG. 3B) binding to mis. WT-TTR_b6 was virtually identical to mis. WT-TTR_b5. NI-301.37F1_3 binding EC₅₀'s for mis. WT-TTR_b5 and b6 were 1.3 and 1.2 nM, respectively. NI-301.37F1_W1 binding EC₅₀'s for mis. WT-TTR_b5 and b6 were 1.0 and 1.4 nM, respectively. Mis. WT-TTR_b6 was also compared to b5 using BLI. NI-301.37F1_3 and NI-301.37F1 W1 bound to mis. WT-TTR_b6 with dissociation constants (KDs) of 0.93 nM and 0.66 nM, respectively. These values were very close to NI-301.37F1_3 binding affinity to mis. WT-TTR_b5, which was measured in a previous experiment with a KD of 0.66 nM. On this basis, mis. WT-TTR_b6 was deemed similar to mis. WT-TTR_b5 and appropriate for use in the ADCP reporter assay.

### ADCP Reporter Assay

To measure the potency of a NI-301.37F1_W1 reference sample (NI-301.37F1_W1 RS) and a half-concentrated test sample (NI-301.37F1_W1 50%) to activate phagocytosis of mis. WT-TTR a commercially available FcγRI ADCP reporter bioassay (Promega, Madison, Wisconsin, USA; early access (not yet validated), CS1781C08) has been applied. This bioluminescent cell-based assay relies on a genetically engineered Jurkat T cell line that expresses the human FcγRI together with a luciferase reporter driven by an NFAT-response element. FcγRI activation by the antibody-target complex leads to activation of NFAT pathway signaling and luciferase expression that is detected using a bioluminescent luciferase substrate. In brief, 96-well plates were coated for 1 hour at 37° C. with mis. WT-TTR diluted to a concentration of 10 μg/ml in PBS buffer pH 7.4. Non-specific binding sites were blocked for 1 hour at room temperature (RT) with a blocking buffer containing 2% bovine serum albumin (BSA) and 0.1% tween-20 in PBS buffer. NI-301.37F1_W1 antibody was diluted in triplicates to the indicated concentrations in PBS and incubated 30 min at 37° C. prior addition of the FcγRI reporter cells at a density of 77,000 cells/well. The assay was incubated for 6 hours at 37° C. before addition of the luminescent substrate.

NI-301.37F1 W1 RS vs. NI-301.37F1 W1 50%

In a first experiment, NI-301.37F1 W1 RS was tested using a 10-point concentration range from 2 to 10,000 ng/ml in triplicates. NI-301.37F1 W1 50% was prepared using the same dilution series but starting from a 2-time lower concentration (i.e., 5,000 ng/ml) to mimic loss of potency.

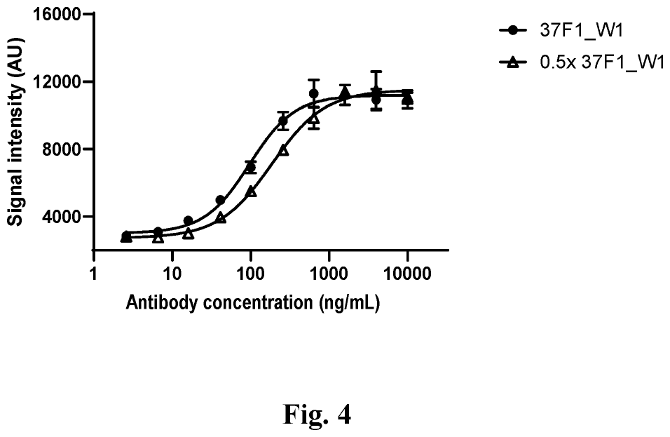

NI-301.37F1_W1 RS presented a dose-response characterized by an EC50 of 97 ng/ml (95% confidence interval (CI) 79.4-116.7). In contrast, NI-301.37F1_W1 50% presented a dose-response characterized by an EC₅₀ of 187 ng/ml (156.2-223.6) (FIG. 4). The EC₅₀ increase by a factor 1.9 was in good agreement with the 2-time lower concentration in sample NI-301.37F1_W1 50%. EC₅₀'s for NI-301.37F1_W1 RS and NI-301.37F1_W1 50% were statistically different (F (1, 52): 26.60, p<0.0001). This result indicated that the FcγR1 ADCP assay had the capacity to detect a 50% loss of antibody activity. The data also indicated that NI-301.37F1_W1 RS dose-response presented 4 data points at the plateau phase. This is unnecessary and triggered an adjustment of the antibody concentration range for following experiments.

A second set of experiments was conducted to: 1) adjust NI-301.37F1 W1 RS concentration range, 2) compare NI-301.37F1_W1 RS to samples with 35% lower and 35% higher concentrations (NI-301.37F1_W1 65% and 135%, respectively), 3) compare horizontal and vertical plate layouts, and 4) test plate uniformity.

NI-301.37F1 W1 RS Vs. NI-301.37F1 W1 65% and 135% Using Horizontal Layout

NI-301.37F1_W1 RS dose-response was adjusted to an 8-point concentration range from 4 to 2000 ng/ml (i.e., 2000, 500, 250, 125, 63, 31, 16, 4 ng/ml) and tested in duplicates using a horizontal plate layout. NI-301.37F1_W1 RS in plate 1 presented a dose-response characterized by an EC₅₀ of 93.7 ng/ml (95% CI 67.4-128.5). In contrast, NI-301.37F1_W1 65% presented a dose-response characterized by an EC₅₀ of 159 ng/ml (118.3-219.4) (FIG. 5A). EC₅₀'s for NI-301.37F1_W1 RS and NI-301.37F1_W1 65% were statistically different (F(1, 24): 7.14, p=0.013). This result indicated that the FcγR1 ADCP assay had the capacity to detect a 35% loss of antibody activity. However, the EC₅₀ increase by a factor 1.7 was a bit far from the expected value of 1.35 for NI-301.37F1_W1 65%.

NI-301.37F1_W1 RS in plate 2 presented a dose-response characterized by an EC₅₀ of 131.5 ng/ml (95% CI 103.2-170.1). In contrast, NI-301.37F1_W1 135% presented a dose-response characterized by an EC₅₀ of 76.7 ng/ml (68.1-86.2) (FIG. 5B). EC₅₀'s for NI-301.37F1_W1 RS and NI-301.37F1_W1 135% were statistically different (F(1, 24): 20.97, p=0.0001). This result indicated that the FcγRI ADCP assay had the capacity to detect a 35% increase in antibody activity. The EC₅₀ decrease by a factor 0.6 was in good agreement with the expected value of 0.65 for NI-301.37F1_W1 135%.

The comparison of dose-responses for NI-301.37F1_W1 RS in plates 1 and 2 revealed a certain difference in maximum signal intensity, which occurred in spite of these plates being run in parallel, on the same day and by the same analyst. This difference illustrated the possible benefit of using a vertical plate layout which would allow 3 samples to be measured in parallel on the same plate and in triplicates.

### NI-301.37F1 W1 RS Vs. 65% and 135% Using Vertical Layout

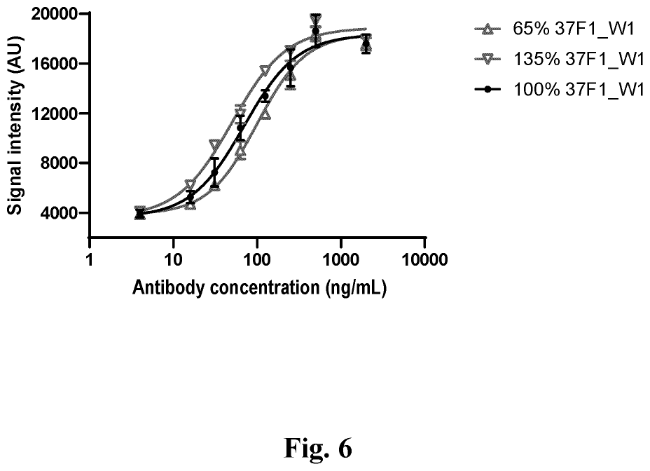

The vertical assay layout was evaluated using the same concentration range as above and triplicate samples. NI-301.37F1_W1 RS in the vertical assay layout presented a dose-response characterized by an EC₅₀ of 70.0 ng/ml (95% CI 53.2-91.5), NI-301.37F1_W1 65% an EC₅₀ of 99.5 ng/ml (82.7-119.1), and NI-301.37F1 W1 135% an EC₅₀ of 50.3 ng/ml (41.1-60.8) (FIG. 6). The EC₅₀ increase by a factor 1.4 for NI-301.37F1_W1 65%, and the EC₅₀ decrease by a factor 0.7 for NI-301.37F1_W1 135% were in good agreement with the respective sample concentrations. NI-301.37F1_W1 RS and NI-301.37F1_W1 65% presented statistically different EC₅₀ values (F (1, 40): 5.199, p-0.028), as well as NI-301.37F1_W1 RS and NI-301.37F1_W1 135% (F (1, 40): 4.358, p-0.043). This indicated that the FcγRI ADCP assay using the vertical format had the capacity to detect changes in antibody activity by ±35%.

### Plate Uniformity Test

A plate uniformity evaluation was conducted using NI-301.37F1_W1 at 12 ng/ml in all 96 wells of the plate. The 24 outside wells presented a signal intensity which was on average 5% lower than the one measured with the 60 inside wells. This small difference was statistically significant. In addition, all wells yielded a sufficiently reliable result, though the 24 outside wells presented a larger variability than the one observed with the 60 inner wells.

### Example 4: Evaluation of FcγR1 ADCP Assay Using Stressed NI-301.37F1_W1 Samples

In order to further evaluate the ADCP assay depicted in Example 3, antibody NI-301.37F1_W1 has been subjected to stress conditions known to potentially cause a loss in antibody potency.

Stressed NI-301.37F1_W1 samples were prepared by dialyzing NI-301.37F1_W1 at 25 mg/ml into five different buffers, listed thereafter. The dialysis was performed overnight at 4° C. and followed by an incubation at 40° C. for 19 hours. The stressed samples were then dialyzed back to formulation buffer overnight at 4° C. prior to aliquoting and storage at −20° C. The buffers used to prepare stressed samples were:
acidic buffer: 20 mM Phosphate buffer-Citric acid (PBCA) buffer, pH3.4
formulation buffer: 20 mM Histidine-HCl, 7% sucrose, 0.02% PS80, pH5.8 (Formº buffer)
physiological buffer: PBS, pH7.4
basic buffer: 20 mM Tris-HCl, pH10.0
oxidative buffer: 1% H₂O₂ in PBS

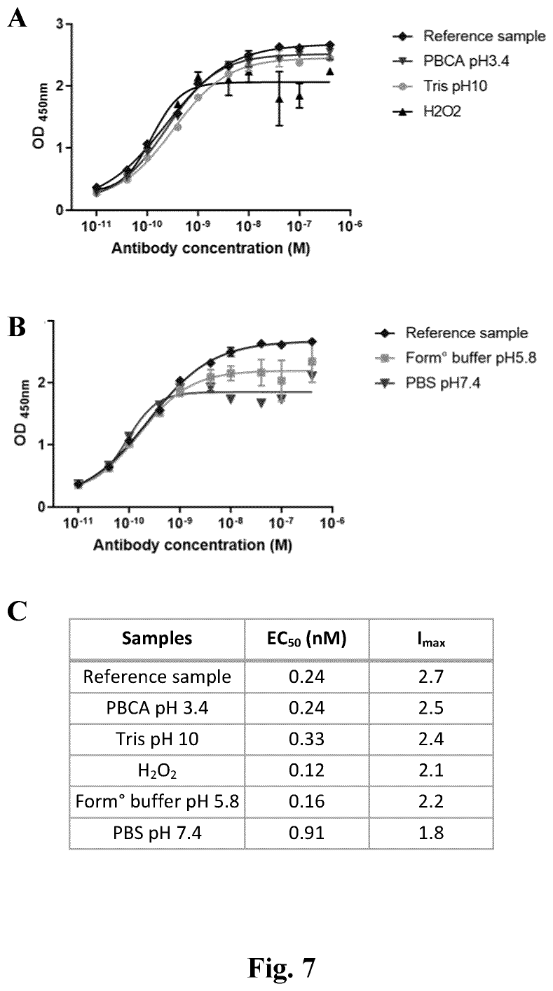

Stressed NI-301.37F1_W1 samples were characterized by measuring their binding affinity to mis. WT-TTR using ELISA and BLI as described above. In addition, stressed NI-301.37F1_W1 samples were characterized using SDS-PAGE under reducing and non-reducing conditions and silver stain according to standard techniques to identify possible aggregation or degradation products. In the ELISA, stressed NI-301.37F1_W1 samples presented binding affinities for mis. WT-TTR which were highly comparable to the reference NI-301.37F1_W1 sample and characterized by EC₅₀'s in the sub-nanomolar range (FIG. 7). The samples stressed in PBS buffer, 1% hydrogen peroxide, and to a lesser extent in formulation buffer, presented lower maximum signal intensity than the reference sample.

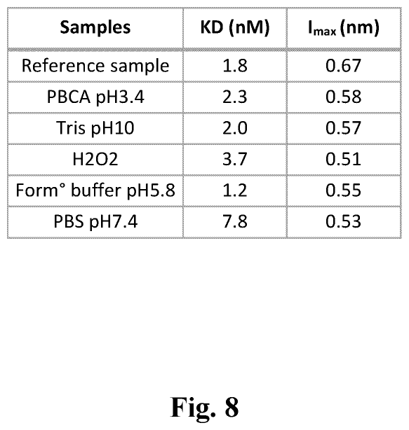

Similar results were obtained using BLI and a summary of the binding results obtained by BLI is depicted in FIG. 8. Stressed NI-301.37F1_W1 samples presented binding affinities for mis. WT-TTR which were comparable to the reference NI-301.37F1_W1 sample and characterized by KDs in the low nanomolar range. As for ELISA, the samples stressed in PBS buffer or 1% hydrogen peroxide presented lower maximum signal intensity than the reference sample. Using SDS-PAGE and silver stain, samples stressed in formulation, phosphate and tris buffers presented patterns under reducing and non-reducing conditions that were like the reference sample. The NI-301.37F1_W1 sample stressed in PBCA buffer (pH 3.4) presented cleaved forms which were visible under reducing and non-reducing conditions, and the NI-301.37F1_W1 sample stressed in 1% H₂O₂ presented under non-reducing conditions a pattern clearly different from the reference sample.

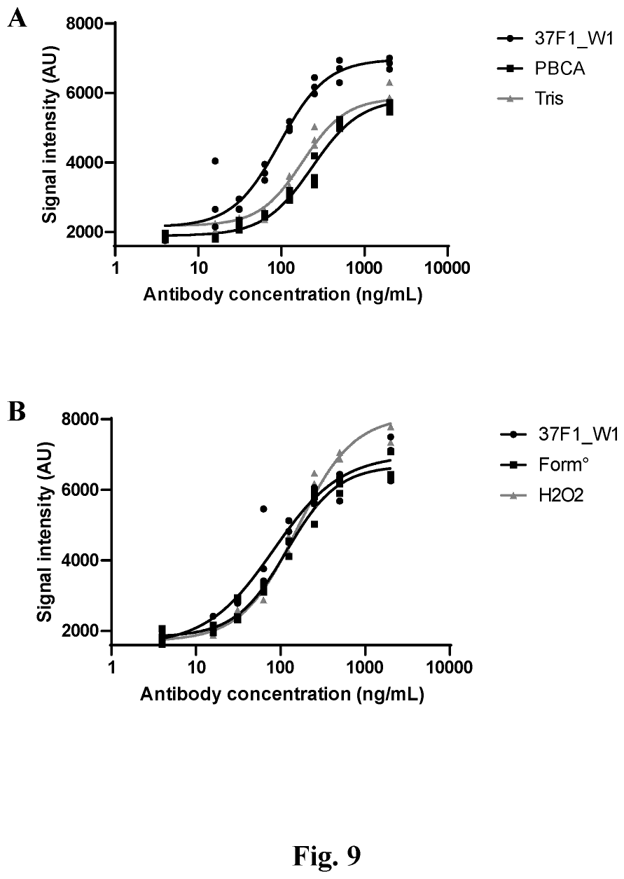

The FcγRI ADCP assay was performed as described in Example 3 using the stressed NI-301.37F1_W1 samples, with the goal of evaluating if this assay had the capacity to detect potency loss. The vertical assay layout was used with samples in triplicates. In assay plate 1, NI-301.37F1_W1 RS presented a dose-response characterized by an EC₅₀ of 95 ng/ml (95% CI 67-127), NI-301.37F1_W1 stressed in PBCA buffer an EC₅₀ of 235 ng/ml (191-292), and NI-301.37F1_W1 stressed in Tris buffer an EC₅₀ of 180 ng/ml (120-385) (FIG. 9A). In assay plate 2, NI-301.37F1_W1 RS presented a dose-response characterized by an EC₅₀ of 83 ng/ml (95% CI 52-138), NI-301.37F1_W1 stressed in formulation buffer an EC₅₀ of 117 ng/ml (96-144), and NI-301.37F1 W1 stressed in H₂O₂ buffer an EC₅₀ of 158 ng/ml (127-200) (FIG. 9B).

Considering that the stressed samples presented binding affinities similar to the NI-301.37F1_W1 RS in the ELISA and BLI assays, these results indicated that the FcγRI ADCP assay had the capacity to detect potency loss related to Fc domain alterations.

### Example 5: Cyclic Peptide as Target Antigen Provides for Higher Sensitivity of ELISA Assay for an Antigen Binding Molecule

The ability of an antigen binding molecule to bind a cyclic peptide has exemplarily been evaluated with an ELISA assay using a cyclic peptide comprising the amino acid residues 34 to 54 of wild type TTR (TTR34-54cyc in biotinylated and non-biotinylated form) as target antigen and the anti-TTR antibody NI-301.37F1 as antigen binding molecule. Furthermore, as antigen controls, the TTR peptide TTR40-49, the biotinylated TTR peptide TTR40-49 as well as misfolded wild type TTR (mis. WT-TTR) were used.

The cyclic peptide TTR34-54cyc (1.36 mg/mL) has been manufactured by Schafer-N (Copenhagen, Denmark) and stored at −20° C. In particular, the peptide comprising the amino acid sequence H-GCGGGRKAADDTWEPFASGKTSESGEGGGCG-OH (SEQ ID NO: 17) has been synthesized by solid phase peptide synthesis and cyclized via disulfide bridge between two cysteine residues within the poly-glycine stretch. The TTR peptide comprising the amino acid sequence H-TWEPFASGKT-OH (SEQ ID NO: 161) (TTR40-49, 1.25 mg/mL) has also been manufactured by Schafer-N(Copenhagen, Denmark) and stored at −20° C. The biotinylated peptides Biotin. TTR34-54cyc and Biotin. TTR40-49 each comprise an amino hexanoic acid (Ahx) spacer between their N-terminus and the biotin residue, i.e., Biotin.TTR34-54cyc (Biotin-(Ahx) GCGGGRKAADDTWEPFASGKTSESGEGGGCG-OH (SEQ ID NO: 17), 680 μg/mL) and Biotin.TTR40-49 (Biotin-(Ahx) TWEPFASGKT-OH, (SEQ ID NO: 161), 700 μg/mL). The misfolded wild type TTR has been prepared as described in Example 3, supra.

Two ELISA assays have been performed, wherein in the first ELISA assay (ELISA-1), antibody binding to the peptides TTR34-54cyc, TTR40-49, Biotin. TTR40-449, and mis-WT-TTR has been analyzed, and in the second ELISA assay (ELISA-2), antibody binding to the peptides TTR34-54cyc, Biotin. TTR34-54cyc, TTR40-49, Biotin. TTR40-449, and mis-WT-TTR has been analyzed.

In particular, 96-well microplates were coated for 1 hour at 37° C. with TTR34-54cyc, TTR40-49, Biotin. TTR40-449, and mis-WT-TTR (FLISA-1) and with TTR34-54cyc, Biotin. TTR34-54cyc, TTR40-49, Biotin. TTR40-449, and mis-WT-TTR (ELISA-2), respectively, wherein each target antigen has been diluted to a concentration of 10 μg/ml in PBS buffer pH 7.4. Non-specific binding sites were blocked for 1 hour at room temperature (RT) with a blocking buffer containing 2% bovine serum albumin (BSA) and 0.1% tween-20 in PBS buffer. NI-301.37F1 antibody (Neurimmune AG, Zurich, Switzerland; NI-301.37F1) was diluted in duplicates to the indicated concentrations (dilution series from 400 nM to 4 PM and 0) in the blocking buffer and incubated overnight at 4° C. Binding was determined using an anti-human IgG antibody conjugated with horseradish peroxidase (HRP), followed by measurement of HRP activity in a standard colorimetric assay (ThermoFisher Scientific Inc., Waltham, Massachusetts, USA). Data were analyzed with the Prism software from GraphPad. ECso values were estimated using non-linear regression of individual data points using log (agonist) versus response model with variable slope. Data fitting was performed with the least square regression method.

The ELISA results confirmed binding of NI-301.37F1 to mis. WT-TTR as also observed in Example 3, supra. Furthermore, the ELISA assay showed that NI-301.37F1 binding to the cyclic TTR34-54cyc and Biotin. TTR34-54cyc peptide is much stronger, i.e., about 10-fold stronger, than binding to mis. WT-TTR. In particular, in ELISA-1 NI-301.37F1 binding EC50 for the cyclic TTR34-54cyc peptide was 27 pM and NI-301.37F1 binding ECso for the mis. WT-TTR was 338 pM; see FIG. 10A. In ELISA-2, the measured FC50 values were higher, but the about 10-fold difference between NI-301.37F1 binding to the cyclic TTR34-54cyc peptide and binding to mis. WT-TTR was maintained. In particular, NI-301.37F 1 binding ECso for the cyclic TTR34-54cyc peptide was 0.66 nM and NI-301.37F1 binding ECso for the mis. WT-TTR was 8.3 nM; see FIG. 10B. No binding of NI-301.37F1 to TTR40-49 and Biotin. TTR40-49 was observed in both ELISA assays.

### Example 6: Improved ADCP Assay by Use of a Cyclic Peptide as Target Antigen

An ADCP assay for determining the potency of an antigen binding molecule was developed. The assay uses a reporter cell line expressing the human Fcγ receptor 1 (FcγR1) and has exemplary been evaluated for its capacity to measure the potency of antibody NI-301.37F1 to activate phagocytosis of a cyclic TTR peptide (TTR34-54cyc) in vitro.

### ADCP Reporter Assay

To measure the potency of a NI-301.37F1 reference sample (NI-301.37F1 RS, 100%) and test samples with 50% lower concentration (NI-301.37F1 50%), 30% lower concentration (NI-301.37F 1 70%), 30% higher concentration (NI-301.37F1 130%), and 50% higher concentration (NI-301.37F1 150%) to activate phagocytosis of TTR34-54cyc, the commercially available FcγRI ADCP reporter bioassay (Promega, Madison, Wisconsin, USA, Cat. #GA1341, GA1345, which is the same as the one described in Example 3 as early access, CS1781C08) has been applied as described in Example 3 with slight variations. In brief, 96-well plates were coated over night at 4° C. with TTR34-54cyc diluted to a concentration of 3 μg/ml in PBS buffer. Non-specific binding sites were blocked for 1 hour at room temperature (RT) with a blocking buffer containing 2% bovine serum albumin (BSA) and 0.1% tween-20 in PBS buffer. A dilution plate for measurement was prepared, wherein NI-301.37F1 antibody was diluted to the indicated concentrations (500 ng/mL to 0.4 ng/mL) in ADCP buffer (96% RPMI 1640 Medium, 4% Low IgG Serum). The assay was performed by adding one unit of volume of the antibody dilutions and incubation was performed for 30 min at 37° C. and 5% CO2 prior addition of one unit of volume of the FcγR 1 reporter cells at a density of about 1.65×10\hat{ }5 cells/well) in ADCP buffer. The assay was incubated for 6 hours at 37° C. and 5% CO2 before addition of the luminescent substrate (Bio-GloTM Luciferase Assay Reagent). The measurement of luminescence (integration time: 1000 ms, settle time: 0 ms) was performed after 15 min of incubation at room temperature.

NI-301.37F1 RS vs. NI-301.37F1 50%, NI-301.37F1 70%, NI-301.37F1 130%, and NI-301.37F1 150%

In a first experiment it was shown that antibody NI-301.37F1 RS presented a dose-response characterized by an ECso of 19.8 ng/ml. In particular, the plates were coated with 3 μg/mL of the cyclic peptide TTR34-54cyc and antibody dilutions ranging from 500-0.4 ng/ml have been tested. These conditions resulted in a reasonable response curve with a stable slope, lower and upper asymptote; see FIG. 11.

Further experiments have been performed in which the response of the assay was tested with 50%, 70%, 130% and 150% NI-301.37F1 concentration. As indicated in FIG. 12A-D and in Table 3 below, NI-301.37F1 50% presented a dose-response characterized by an EC₅₀ of 39 ng/ml. The EC₅₀ increase by a factor of 1.97 was nearly in perfect agreement with the 2-time lower concentration in sample NI-301.37F 50%. NI-301.37F1 70% presented a dose-response characterized by an EC₅₀ of 30.4 ng/ml. The EC₅₀ increase by a factor of 1.43 was in very good agreement with the expected difference of 1.54 times. NI-301.37F1 130% presented a dose-response characterized by an EC₅₀ of 13.6 ng/ml. The EC₅₀ increase by a factor of 0.77 was in perfect agreement with the expected difference of 0.77 times. NI-301.37F1 150% presented a dose-response characterized by an EC₅₀ of 13.5 ng/ml. The EC₅₀ increase by a factor of 0.67 was nearly in perfect agreement with the expected difference of 0.70 times.

Accordingly, there are nearly no assay variabilities and the results showed that the assay is responding to antibody concentration changes. In particular, it was shown that the FcγR1 ADCP assay had the capacity to detect up to 50% loss of antibody activity, and up to 50% increase in antibody activity with excellent accuracy.

TABLE 3
Summary of the FcγR1 ADCP assay performance.

| | Reference | | | | | Expected |
|---|---|---|---|---|---|---|
| | samples | Test samples (TS) | | | | results |
| | (RS) 100% | 50% | 70% | 130% | 150% | (ng/mL) |
| EC50 | 19.8 | 39 | | | | 39.6 |
| (ng/mL) | 19.8 | | 30.4 | | | 28.3 |
| | 17.6 | | | 13.6 | | 13.5 |
| | 19.3 | | | | 13.5 | 12.9 |

### Example 7: Evaluation of Further Cyclic Peptides as Target Antigen for an Antigen Binding Molecule

As shown in Example 5, the cyclic peptide TTR34-54cyc has been successfully used as target antigen for antibody NI-301.37F1 in an ELISA assay. Accordingly, the ability of an anti-TTR antibody to bind further cyclic peptides is analyzed. In particular, the ability of an anti-TTR antibody to bind the two cyclic peptides which comprise either the TTR epitope EHAEVVFTA (SEQ ID NO: 8) or the TTR epitope GPRRYTIAA (SEQ ID NO: 9), i.e., TTR89-97cyc and TTR101-109cyc as mentioned above, is evaluated with a further ELISA assay with said cyclic peptides as target antigens and the with the TTR peptide TTR40-49, the biotinylated TTR peptide TTR40-49 as well as misfolded wild type TTR (mis. WT-TTR) as antigen controls. The corresponding peptides and the mis. WT-TTR are prepared as described in Example 5, supra. For the ELISA assays, the 96-well microplates are coated with the two cyclic peptides TTR89-97cyc and TTR 101-109cy and the antigen controls, and the assay is performed as described in Example 5, supra.

### Example 8: ADCP Assay Using FcγRI Reporter Cell Line for Measuring the Potency of an Antigen Binding Molecule to Activate Phagocytosis of Further Cyclic Peptides

As shown in Example 6, the potency of the anti-TTR antibody NI-301.37F1 to activate phagocytosis of a cyclic TTR peptide (TTR34-54cyc) in vitro has been successfully determined with an ADCP assay. Accordingly, the potency of an anti-TTR antibody to activate phagocytosis of the two cyclic peptides TTR89-97cyc and TTR101-109cyc is evaluated in a further ADCP assay.

To measure the potency of an anti-TTR antibody reference sample (anti-TTR antibody RS, 100%) and test samples with 50% lower concentration (anti-TTR antibody 50%), 30% lower concentration (anti-TTR antibody 70%), 30% higher concentration (anti-TTR antibody 130%), and 50% higher concentration (anti-TTR antibody 150%) to activate phagocytosis of said two cyclic peptides, the commercially available FcγRI ADCP reporter bioassay (Promega, Madison, Wisconsin, USA, Cat. #GA1341, GA1345) as described in Example 6 has been applied.

It is expected that the anti-TTR antibody will presented a dose-response, wherein anti-TTR antibody 50% will show an about 2-fold increased ECso value in comparison to the anti-TTR antibody RS, the anti-TTR antibody 70% will show an about 1.5-fold increased EC₅₀ value in comparison to the anti-TTR antibody RS, anti-TTR antibody 130% will show a decreased ECso value by a factor of about 0.77 in comparison to the anti-TTR antibody RS, and the anti-TTR antibody 150% will show a decreased ECso value by a factor of about 0.70 in comparison to the anti-TTR antibody RS.

1. A method for determining the potency of a target antigen binding molecule comprising an Fc domain comprising the steps of:
(a) contacting a target antigen with the binding molecule under conditions allowing the formation of a binding molecule-antigen complex;
(b) contacting the binding molecule-antigen complex with a population of effector cells that are engineered to express an Fc receptor and harbor a reporter gene under the control of a response element that is responsive to activation by the Fc receptor under conditions allowing for binding of the Fc domain to the Fc receptor, wherein binding of the Fc domain to the Fc receptor results in intracellular signaling and mediates a quantifiable reporter gene activity; and
(c) detecting the reporter gene activity,
wherein at least one mechanism of action of the Fc domain of the binding molecule is mediated through the binding of the Fc domain to a Fc receptor and the reporter gene activity is indicative for the potency of the binding molecule.

2. A method of producing a pharmaceutical composition of a target antigen binding molecule comprising an Fc domain comprising the steps of:
(a) providing, optionally producing said binding molecule;
(b) subjecting said binding molecule to a method of claim 1 for determining the potency of the binding molecule; and
(c) using the information obtained in step (b) as part of an assessment of whether the binding molecule may be used as a pharmaceutical composition or not; and optionally
(d) formulating the binding molecule found to be useful as a pharmaceutical composition in step (c) with a pharmaceutically acceptable carrier.

3. The method according to claim 1 or 2, wherein the target antigen is selected or derived from an amyloidogenic protein involved in systemic amyloidosis or aggregate thereof.

4. The method according to any one of claims 1 to 3, wherein a mechanism of action of the Fc domain is to induce antibody-dependent cell-mediated phagocytosis (ADCP).

5. The method according to any one of claims 1 to 4, wherein the method is used as a potency assay for batch release.

6. The method according to any one of claims 1 to 5, wherein the Fc receptor is a human Fc receptor FcγRI (CD64).

7. The method according to any one of claims 1 to 6, wherein the cell does not overexpress FcγRIIa (CD32a).

8. The method according to any one of claims 1 to 7, wherein the cell does not overexpress FcγRIII (CD16).

9. The method according to any one of claims 1 to 8, wherein the effector cell is a Jurkat cell.

10. The method according to any one of claims 1 to 9, wherein the response element is an NFAT (Nuclear Factor of Activated T cells) response element.

11. The method according to any one of claims 1 to 10, wherein the reporter gene encodes a bioluminescent protein, preferably a luciferase.

12. The method according to any one of claims 1 to 11, wherein the binding molecule is selected or derived from an antibody such as a monoclonal antibody or an antigen-binding fragment thereof, preferably wherein the antibody is a human antibody, a humanized antibody or a chimeric antibody.

13. The method according to claim 12, wherein the antibody is an IgG1 antibody, such as an IgG1, λ antibody or an IgG1, κ antibody.

14. The method of any one of claims 1 to 13, wherein the target antigen comprises transthyretin (TTR) or an aggregate or derivative thereof.

15. The method of any one of claims 1 to 14, wherein the binding molecule is an anti-TTR antibody.

16. The method of any one of claims 1 to 15, wherein the target antigen comprises a protein fragment or peptide comprising an epitope of the target antigen binding molecule.

17. The method of claim 16, wherein the protein fragment or peptide forms a cyclic compound.

18. The method of claim 16 or 17, wherein the protein fragment or peptide comprises a linker that is capable of forming the cyclic compound.

19. The method of claim 18, wherein the linker is covalently coupled at or near the peptide N-terminus residue and the C-terminus residue of the peptide fragment.

20. The method of any one of claims 16 to 19, wherein the target antigen comprises a (neo) epitope of an amyloidogenic protein involved in systemic amyloidosis or aggregate thereof.

21. The method of any one of claims 17 to 19 wherein the protein fragment or peptide in the cyclic compound comprises at least 5, preferably at least 10, more preferably at least 15, most preferably at least 20, 21, 22, 23, 24, or 25 amino acid residues of an amyloidogenic protein.

22. The method of claim 21, wherein the amyloidogenic protein is selected from the group consisting of transthyretin (TTR), immunoglobulin light chain (LC), immunoglobulin heavy chain (LH), serum amyloid A (SAA), leucocyte chemotactic factor 2 (LECT2), gelsolin, apolipoprotein AI (ApoAI), apolipoprotein AII (ApoAII), apolipoprotein AIV (ApoAIV), apolipoprotein CII (ApoCII), apolipoprotein CIII (ApoCIII), Fibrinogen, β2 microglobulin, cystatin C, ABriPP, prion protein, and lysozyme.

23. The method of claim 22, wherein the amyloidogenic protein is TTR and the target antigen comprises a TTR peptide.

24. The method of claim 23, wherein the TTR peptide comprises at least 4 amino acid residues of any one of the amino acid sequences selected from: WEPFA (SEQ ID NO: 1), EEFXEGIY (SEQ ID NO: 2), ELXGLTXE (SEQ ID NO: 3), WEPFASG (SEQ ID NO: 4), TTAVVTNPKE (SEQ ID NO: 5), KCPLMVK and VFRK (SEQ ID NOs: 6 and 7), EHAEVVFTA (SEQ ID NO: 8), GPRRYTIAA (SEQ ID NO: 9), VHVFRKAADDTWEPFASGKTSESGELHGLTTEEEFVE (SEQ ID NO: 10), ALLSPYSYSTTAV (SEQ ID NO: 11), WKALGISPFHE (SEQ ID NO: 12), SYSTTAVVTN (SEQ ID NO: 13), and LLSPYSYSTTAVVTNPKE (SEQ ID NO: 14), wherein X can be any naturally occurring amino acid.

25. The method of claim 24, wherein the TTR peptide comprises the amino acid sequence WEPFA (SEQ ID NO: 1).

26. The method of any one of claims 17 to 25, wherein the linker comprises or consists of 1-8 amino acids and/or one or more functionalizable moieties.

27. The method of claim 26, wherein the linker amino acids are selected from alanine (A), glycine (G), and/or serine(S), and/or wherein the functionalizable moiety is cysteine (C), lysine (K), arginine (R), aspartic acid (D), or glutamic acid (E).

28. The method of claim 27, wherein the functionalizable moiety is cysteine (C) and the compound is cyclized via a disulfide bridge.

29. The method of any one of claims 17 to 28, wherein the linker in the cyclic compound comprises or consists of GCGGG (SEQ ID NO: 15) or GGGCG (SEQ ID NO: 16).

30. The method of any one of claims 17 to 29, wherein the cyclic compound comprises or consists of the amino acid sequence H-GCGGGRKAADDTWEPFASGKTSESGEGGGCG-OH (TTR34-54cyc; SEQ ID NO: 17).

31. The method of any one of claims 1 to 30, wherein the binding molecule is an anti-TTR antibody which is NI-301.37F1 and which comprises in its variable region or binding domain the amino acid sequences of the VH and VL chain of SEQ ID NO: 19 and SEQ ID NO: 21 or SEQ ID NO: 23 and SEQ ID NO: 21.

32. The method according to any one of claims 1 to 31, wherein the target antigen is bound on a solid support, preferably on a microtiter plate.

33. The method according to claim 32, wherein at least step (b) of claim 1 is performed in a vertical plate layout.

34. Use of a target antigen binding molecule and/or an effector cell that is engineered to express a human Fc receptor FcγRI (CD64) and harbors a reporter gene under the control of a response element that is responsive to activation by the Fc receptor in the method according to any one of claims 1 to 33.

35. A method for analyzing and selecting at least one batch of a pharmaceutical composition of a target antigen binding molecule as defined in any one of the preceding claims, the method comprising the steps of:
(a) subjecting a sample of the batch to a method according to any one of claims 1 to 33:
(b) comparing the reporter gene activity of the sample to the reporter gene activity of a control; and
(c) selecting the batch for which the sample shows greater, equal or no less than 80% reporter gene activity compared to the control, preferably wherein the control is a reference standard and/or the batch to be analyzed has been stored and/or was subjected to stress conditions and the control is the value of reporter gene activity of a sample taken from the batch or corresponding batch prior to storage and/or being subjected to said stress conditions.

36. A kit useful in a method according to any one of the preceding claims, wherein the kit comprises:
(i) a population of effector cells engineered to express a human Fc receptor FcγRI (CD64) and harbor a reporter gene under the control of a response element that is responsive to activation by the Fc receptor, preferably wherein the population of effector cells is a population of Jurkat cells and the reporter gene encodes a luminescence protein, preferably a luciferase under control of NFAT response element;
(ii) a corresponding substrate for the reporter; preferably further comprising one or more of the following:
(iii) the target antigen, preferably wherein the target antigen is an aggregated protein or protein prone to aggregation, and/or a cyclic compound comprising the epitope of a target binding molecule, or a linear precursor of the cyclic compound;
(iv) a microtiter plate, preferably a 96-well plate including a lid;
(v) a buffer, diluent, substrate and/or solution;
(vi) washing, blocking and assay/sample dilution buffer;
(vii) a monomer control of the target antigen and/or a positive control anti-target antigen antibody; and/or
(viii) instructions for use.

37. The kit of claim 36, which comprise the population of effector cells of (i), the corresponding substrate of (ii), and the target antigen of (iii), wherein the target antigen is an amyloidogenic protein involved in systemic amyloidosis, preferably wherein the kit further comprises one or more of (iv) to (viii).

38. A cyclic compound comprising a peptide comprising an epitope from an amyloidogenic protein involved in systemic amyloidosis.

39. The cyclic compound of claim 38, which comprises a linker, preferably wherein the linker is an amino acid linker or a non-amino acid linker.

40. The cyclic compound of claim 39, wherein the linker is covalently coupled at or near the peptide N-terminus residue and the C-terminus residue of the peptide.

41. The cyclic compound of any one of claims 38 to 40, wherein the peptide in the cyclic compound comprises at least 5, preferably at least 10, more preferably at least 15, most preferably at least 20, 21, 22, 23, 24, or 25 amino acid residues of the amyloidogenic protein.

42. The cyclic compound of any one of claims 38 to 41, wherein the amyloidogenic protein is selected from transthyretin (TTR), immunoglobulin light chain (LC), and serum amyloid A (SAA).

43. The cyclic compound of claim 42, wherein the amyloidogenic protein is TTR and the peptide is a TTR peptide.

44. The cyclic compound of claim 43, wherein the TTR peptide comprises at least 4 amino acid residues of any one of the amino acid sequences selected from: WEPFA (SEQ ID NO: 1), EEFXEGIY (SEQ ID NO: 2), ELXGLTXE (SEQ ID NO: 3), WEPFASG (SEQ ID NO: 4), TTAVVTNPKE (SEQ ID NO: 5), KCPLMVK and VFRK (SEQ ID NOs: 6 and 7), EHAEVVFTA (SEQ ID NO: 8), GPRRYTIAA (SEQ ID NO: 9), VHVFRKAADDTWEPFASGKTSESGELHGLTTEEEFVE (SEQ ID NO: 10), ALLSPYSYSTTAV (SEQ ID NO: 11), WKALGISPFHE (SEQ ID NO: 12), SYSTTAVVTN (SEQ ID NO: 13), and LLSPYSYSTTAVVTNPKE (SEQ ID NO: 14), wherein X can be any naturally occurring amino acid.

45. The cyclic compound of claim 43 or 44, wherein the TTR peptide comprises the amino acid sequence WEPFA (SEQ ID NO: 1).

46. The cyclic compound of any one of claims 38 to 45, wherein the linker comprises or consists of 1-8 amino acids and/or one or more functionalizable moieties.

47. The cyclic compound of claim 46, wherein the linker amino acids are selected from alanine (A), glycine (G), and/or serine(S), and/or wherein the functionalizable moiety is cysteine (C), lysine (K), arginine (R), aspartic acid (D), or glutamic acid (E).

48. The cyclic compound of claim 47, wherein the compound is cyclized via a disulfide bridge.

49. The cyclic compound of any one of claims 37 to 48, wherein the linker in the cyclic compound comprises or consists of the amino acid sequence GCGGG (SEQ ID NO: 15) or GGGCG (SEQ ID NO: 16).

50. The cyclic compound of any one of claims 37 to 49, wherein the cyclic compound comprises or consists of the amino acid sequence H-GCGGGRKAADDTWEPFASGKTSESGEGGGCG-OH (TTR34-54cyc; SEQ ID NO: 17).

51. The cyclic compound of any one of claims 37 to 50, wherein the peptide is further derivatized.

52. A precursor of the cyclic compound of any one of claims 37 to 51, wherein the compound is in linear form.

53. A composition comprising the cyclic compound of any one of claims 37 to 51, and optionally one or more excipients.

54. A kit which comprises at least the cyclic compound of any one of claims 37 to 51 or the precursor of claim 52, optionally with reagents and/or instructions for use.

55. The kit of claim 54, further comprising
(i) a population of effector cells engineered to express an Fc receptor, preferably a human Fc receptor FcγR, and harbor a reporter gene under the control of a response element that is responsive to activation by the Fc receptor, preferably wherein the population of effector cells is a population of Jurkat cells and the reporter gene encodes a luminescence protein, preferably a luciferase under control of NFAT response element;
(ii) a corresponding substrate for the reporter; preferably wherein the kit further comprises one or more of the following:
(iii) a solid support, preferably a microtiter plate, preferably a 96-well plate including a lid;
(iv) washing, blocking and assay/sample dilution buffer; and/or
(v) a monomer control of the target antigen and/or a positive control anti-target antigen antibody.

56. Use of a cyclic compound of any one of claims 36 to 51, or the composition of claim 48, or the kit of claim 54 or 55, for detecting an antigen binding molecule, preferably for determining the potency of an antigen binding molecule comprising an Fc domain.

57. The use of claim 56, wherein determining the potency of the antigen binding molecule is performed in accordance with the method of any one of claims 1 to 33.

58. A method for identifying and optionally obtaining an antibody which binds to an amyloidogenic protein involved in systemic amyloidosis, the method comprising the steps of:
(a) providing, optionally producing one or more potentially amyloidogenic protein binding antibodies or a source thereof;
(b) subjecting the one or more of potentially amyloidogenic protein binding antibodies or source thereof to a binding assay comprising the cyclic compound of any one of claims 36 to 51; and
(c) identifying and optionally obtaining an antibody (subject antibody) that has been determined to bind to the cyclic compound.

59. A method of producing a pharmaceutical composition comprising an antibody which binds to an amyloidogenic protein, the method comprising the steps of:
(a) providing, optionally producing one or more potentially amyloidogenic protein binding antibodies or a source thereof;
(b) subjecting said one or more potentially amyloidogenic protein binding antibodies or a source thereof to a binding assay comprising the cyclic compound of any one of claims 36 to 51;
(c) identifying and optionally obtaining an antibody (subject antibody) that binds to the cyclic compound; and
(d) formulating the antibody identified and optionally obtained in step (c) or a derivative thereof with a pharmaceutically acceptable carrier.

60. The method of claim 58 or 59, wherein the source of antibodies is selected form the group consisting of: immunized laboratory animal such as a rodent, preferably mouse, most preferably Ig humanized mouse; human blood or a fraction thereof, preferably comprising memory B cells; recombinant antibody libraries such as phage, yeast, and ribosome systems or mammalian cell systems such as CHO and HEK.

61. The method of any one of claims 58 to 60, wherein the binding assay comprises ELISA.

62. The method of any one of claims 58 to 61, wherein the antibody identified and optionally obtained in step (c) competes with a reference antibody for binding the amyloidogenic protein, preferably wherein the subject antibody has a lower EC₅₀ for the amyloidogenic protein than the reference antibody.


SEQUENCE LISTING (161 sequences)

SEQ ID NO: 1 - protein, 5 aa, Homo sapiens
WEPFA

SEQ ID NO: 2 - protein, 8 aa, Homo sapiens
EEFXEGIY

SEQ ID NO: 3 - protein, 8 aa, Homo sapiens
ELXGLTXE

SEQ ID NO: 4 - protein, 7 aa, Homo sapiens
WEPFASG

SEQ ID NO: 5 - protein, 10 aa, Homo sapiens
TTAVVTNPKE

SEQ ID NO: 6 - protein, 7 aa, Homo sapiens
KCPLMVK

SEQ ID NO: 7 - protein, 4 aa, Homo sapiens
VFRK

SEQ ID NO: 8 - protein, 9 aa, Homo sapiens
EHAEVVFTA

SEQ ID NO: 9 - protein, 9 aa, Homo sapiens
GPRRYTIAA

SEQ ID NO: 10 - protein, 37 aa, Homo sapiens
VHVFRKAADDTWEPFASGKTSESGELHGLTTEEEFVE

SEQ ID NO: 11 - protein, 13 aa, Homo sapiens
ALLSPYSYSTTAV

SEQ ID NO: 12 - protein, 11 aa, Homo sapiens
WKALGISPFHE

SEQ ID NO: 13 - protein, 10 aa, Homo sapiens
SYSTTAVVTN

SEQ ID NO: 14 - protein, 18 aa, Homo sapiens
LLSPYSYSTTAVVTNPKE

SEQ ID NO: 15 - protein, 5 aa, synthetic construct
GCGGG

SEQ ID NO: 16 - protein, 5 aa, synthetic construct
GGGCG

SEQ ID NO: 17 - protein, 31 aa, synthetic construct
GCGGGRKAADDTWEPFASGKTSESGEGGGCG

SEQ ID NO: 18 - DNA, 360 bp, Homo sapiens
  source 1..360: mol_type = other DNA
caggtgcagctgcaggagtcgggcccaggactggtgaagccttcggagaccctgtccctcacctgcagtgtctctggtggctccatcatcagtaggagttcctactggggctggatccgccagcccccagggaaggggctggagtggattgggggtatctatcatagtgggaacacttacgacaacccgtccctcaagagtcgactcaccatgtccgtagacacgtcgaagaaccagttctccctgaatctgaggtctgtgaccgccgcagacacggctgtgtattactgtgcgaggatagtgccggggggtgatgcttttgatatctggggccaagggacaatggtcaccgtctcttcg

SEQ ID NO: 19 - protein, 120 aa, Homo sapiens
  REGION 52..67: note = Complementarity determining region (CDR) VH-CDR2
  REGION 100..109: note = Complementarity determining region (CDR) VH-CDR3
  REGION 31..35: note = Complementarity determining region (CDR) VH-CDR1
QVQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 20 - DNA, 321 bp, Homo sapiens
  source 1..321: mol_type = other DNA
gacatccagatgacccagtctccatcctccctgtctgcatctgtaggagacagagtcacaatcgcttgccgggccagtcagagcgttggcacctatttaaattggtatcagcagaaaagagggaaagcccctaaactcctcatctttgctgcatccagtttgcaaagtggggtcccatcaaggttcagtggcagtggatctgggacagatttcactctcaccatcagcagtctgcaacctgaagactttgcaacttactactgtcaacagagttacagttctcctccaacgttcggccaagggaccaaggtggagatcaaa

SEQ ID NO: 21 - protein, 107 aa, Homo sapiens
  REGION 24..34: note = Complementarity determining region (CDR) VL-CDR1
  REGION 50..56: note = Complementarity determining region (CDR) VL-CDR2
  REGION 89..97: note = Complementarity determining region (CDR) VL-CDR3
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIK

SEQ ID NO: 22 - DNA, 360 bp, Homo sapiens
  source 1..360: mol_type = other DNA
cagctgcagctgcaggagtcgggcccaggactggtgaagccttcggagaccctgtccctcacctgcagtgtctctggtggctccatcatcagtaggagttcctactggggctggatccgccagcccccagggaaggggctggagtggattgggggtatctatcatagtgggaacacttacgacaacccgtccctcaagagtcgactcaccatgtccgtagacacgtcgaagaaccagttctccctgaatctgaggtctgtgaccgccgcagacacggctgtgtattactgtgcgaggatagtgccggggggtgatgcttttgatatctggggccaagggacaatggtcaccgtctcttcg

SEQ ID NO: 23 - protein, 120 aa, Homo sapiens
  REGION 31..35: note = Complementarity determining region (CDR) VH-CDR1
  REGION 52..67: note = Complementarity determining region (CDR) VH-CDR2
  REGION 100..109: note = Complementarity determining region (CDR) VH-CDR3
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 24 - protein, 4 aa, Homo sapiens
GPTG

SEQ ID NO: 25 - protein, 4 aa, Homo sapiens
TGES

SEQ ID NO: 26 - protein, 4 aa, Homo sapiens
KCPL

SEQ ID NO: 27 - protein, 4 aa, Homo sapiens
MVKV

SEQ ID NO: 28 - protein, 4 aa, Homo sapiens
LDAV

SEQ ID NO: 29 - protein, 4 aa, Homo sapiens
RGSP

SEQ ID NO: 30 - protein, 4 aa, Homo sapiens
AINV

SEQ ID NO: 31 - protein, 4 aa, Homo sapiens
AVHV

SEQ ID NO: 32 - protein, 4 aa, Homo sapiens
FRKA

SEQ ID NO: 33 - protein, 4 aa, Homo sapiens
ADDT

SEQ ID NO: 34 - protein, 4 aa, Homo sapiens
WEPF

SEQ ID NO: 35 - protein, 4 aa, Homo sapiens
ASGK

SEQ ID NO: 36 - protein, 4 aa, Homo sapiens
TSES

SEQ ID NO: 37 - protein, 4 aa, Homo sapiens
GELH

SEQ ID NO: 38 - protein, 4 aa, Homo sapiens
GLTT

SEQ ID NO: 39 - protein, 4 aa, Homo sapiens
EEEF

SEQ ID NO: 40 - protein, 4 aa, Homo sapiens
VEGI

SEQ ID NO: 41 - protein, 4 aa, Homo sapiens
YKVE

SEQ ID NO: 42 - protein, 4 aa, Homo sapiens
IDTK

SEQ ID NO: 43 - protein, 4 aa, Homo sapiens
SYWK

SEQ ID NO: 44 - protein, 4 aa, Homo sapiens
ALGI

SEQ ID NO: 45 - protein, 4 aa, Homo sapiens
SPFH

SEQ ID NO: 46 - protein, 4 aa, Homo sapiens
EHAE

SEQ ID NO: 47 - protein, 4 aa, Homo sapiens
VVFT

SEQ ID NO: 48 - protein, 4 aa, Homo sapiens
ANDS

SEQ ID NO: 49 - protein, 4 aa, Homo sapiens
GPRR

SEQ ID NO: 50 - protein, 4 aa, Homo sapiens
YTIA

SEQ ID NO: 51 - protein, 4 aa, Homo sapiens
ALLS

SEQ ID NO: 52 - protein, 4 aa, Homo sapiens
PYSY

SEQ ID NO: 53 - protein, 4 aa, Homo sapiens
STTA

SEQ ID NO: 54 - protein, 4 aa, Homo sapiens
VVTN

SEQ ID NO: 55 - protein, 4 aa, Homo sapiens
PTGT

SEQ ID NO: 56 - protein, 4 aa, Homo sapiens
GESK

SEQ ID NO: 57 - protein, 4 aa, Homo sapiens
CPLM

SEQ ID NO: 58 - protein, 4 aa, Homo sapiens
VKVL

SEQ ID NO: 59 - protein, 4 aa, Homo sapiens
DAVR

SEQ ID NO: 60 - protein, 4 aa, Homo sapiens
GSPA

SEQ ID NO: 61 - protein, 4 aa, Homo sapiens
INVA

SEQ ID NO: 62 - protein, 4 aa, Homo sapiens
VHVF

SEQ ID NO: 63 - protein, 4 aa, Homo sapiens
RKAA

SEQ ID NO: 64 - protein, 4 aa, Homo sapiens
DDTW

SEQ ID NO: 65 - protein, 4 aa, Homo sapiens
EPFA

SEQ ID NO: 66 - protein, 4 aa, Homo sapiens
SGKT

SEQ ID NO: 67 - protein, 4 aa, Homo sapiens
SESG

SEQ ID NO: 68 - protein, 4 aa, Homo sapiens
ELHG

SEQ ID NO: 69 - protein, 4 aa, Homo sapiens
LTTE

SEQ ID NO: 70 - protein, 4 aa, Homo sapiens
EEFV

SEQ ID NO: 71 - protein, 4 aa, Homo sapiens
EGIY

SEQ ID NO: 72 - protein, 4 aa, Homo sapiens
KVEI

SEQ ID NO: 73 - protein, 4 aa, Homo sapiens
DTKS

SEQ ID NO: 74 - protein, 4 aa, Homo sapiens
YWKA

SEQ ID NO: 75 - protein, 4 aa, Homo sapiens
LGIS

SEQ ID NO: 76 - protein, 4 aa, Homo sapiens
PFHE

SEQ ID NO: 77 - protein, 4 aa, Homo sapiens
HAVE

SEQ ID NO: 78 - protein, 4 aa, Homo sapiens
VFTA

SEQ ID NO: 79 - protein, 4 aa, Homo sapiens
NDSG

SEQ ID NO: 80 - protein, 4 aa, Homo sapiens
PRRY

SEQ ID NO: 81 - protein, 4 aa, Homo sapiens
TIAA

SEQ ID NO: 82 - protein, 4 aa, Homo sapiens
LLSP

SEQ ID NO: 83 - protein, 4 aa, Homo sapiens
YSYS

SEQ ID NO: 84 - protein, 4 aa, Homo sapiens
TTAV

SEQ ID NO: 85 - protein, 4 aa, Homo sapiens
VTNP

SEQ ID NO: 86 - protein, 4 aa, Homo sapiens
TGTG

SEQ ID NO: 87 - protein, 4 aa, Homo sapiens
ESKC

SEQ ID NO: 88 - protein, 4 aa, Homo sapiens
PLMV

SEQ ID NO: 89 - protein, 4 aa, Homo sapiens
KVLD

SEQ ID NO: 90 - protein, 4 aa, Homo sapiens
AVRG

SEQ ID NO: 91 - protein, 4 aa, Homo sapiens
SPAI

SEQ ID NO: 92 - protein, 4 aa, Homo sapiens
NVAV

SEQ ID NO: 93 - protein, 4 aa, Homo sapiens
HVFR

SEQ ID NO: 94 - protein, 4 aa, Homo sapiens
KAAD

SEQ ID NO: 95 - protein, 4 aa, Homo sapiens
DTWE

SEQ ID NO: 96 - protein, 4 aa, Homo sapiens
PFAS

SEQ ID NO: 97 - protein, 4 aa, Homo sapiens
GKTS

SEQ ID NO: 98 - protein, 4 aa, Homo sapiens
ESGE

SEQ ID NO: 99 - protein, 4 aa, Homo sapiens
LHGL

SEQ ID NO: 100 - protein, 4 aa, Homo sapiens
TTEE

SEQ ID NO: 101 - protein, 4 aa, Homo sapiens
EFVE

SEQ ID NO: 102 - protein, 4 aa, Homo sapiens
GIYK

SEQ ID NO: 103 - protein, 4 aa, Homo sapiens
VEID

SEQ ID NO: 104 - protein, 4 aa, Homo sapiens
TKSY

SEQ ID NO: 105 - protein, 4 aa, Homo sapiens
WKAL

SEQ ID NO: 106 - protein, 4 aa, Homo sapiens
GISP

SEQ ID NO: 107 - protein, 4 aa, Homo sapiens
FHEH

SEQ ID NO: 108 - protein, 4 aa, Homo sapiens
AEVV

SEQ ID NO: 109 - protein, 4 aa, Homo sapiens
FTAN

SEQ ID NO: 110 - protein, 4 aa, Homo sapiens
DSGP

SEQ ID NO: 111 - protein, 4 aa, Homo sapiens
RRYT

SEQ ID NO: 112 - protein, 4 aa, Homo sapiens
IAAL

SEQ ID NO: 113 - protein, 4 aa, Homo sapiens
LSPY

SEQ ID NO: 114 - protein, 4 aa, Homo sapiens
SYST

SEQ ID NO: 115 - protein, 4 aa, Homo sapiens
TAVV

SEQ ID NO: 116 - protein, 4 aa, Homo sapiens
TNPK

SEQ ID NO: 117 - protein, 4 aa, Homo sapiens
GTGE

SEQ ID NO: 118 - protein, 4 aa, Homo sapiens
SKCP

SEQ ID NO: 119 - protein, 4 aa, Homo sapiens
LMVK

SEQ ID NO: 120 - protein, 4 aa, Homo sapiens
VLDA

SEQ ID NO: 121 - protein, 4 aa, Homo sapiens
VRGS

SEQ ID NO: 122 - protein, 4 aa, Homo sapiens
PAIN

SEQ ID NO: 123 - protein, 4 aa, Homo sapiens
VAVH

SEQ ID NO: 124 - protein, 4 aa, Homo sapiens
VFRK

SEQ ID NO: 125 - protein, 4 aa, Homo sapiens
AADD

SEQ ID NO: 126 - protein, 4 aa, Homo sapiens
TWEP

SEQ ID NO: 127 - protein, 4 aa, Homo sapiens
FASG

SEQ ID NO: 128 - protein, 4 aa, Homo sapiens
KTSE

SEQ ID NO: 129 - protein, 4 aa, Homo sapiens
SGEL

SEQ ID NO: 130 - protein, 4 aa, Homo sapiens
HGLT

SEQ ID NO: 131 - protein, 4 aa, Homo sapiens
TEEE

SEQ ID NO: 132 - protein, 4 aa, Homo sapiens
FVEG

SEQ ID NO: 133 - protein, 4 aa, Homo sapiens
IYKV

SEQ ID NO: 134 - protein, 4 aa, Homo sapiens
EIDT

SEQ ID NO: 135 - protein, 4 aa, Homo sapiens
KSYW

SEQ ID NO: 136 - protein, 4 aa, Homo sapiens
KALG

SEQ ID NO: 137 - protein, 4 aa, Homo sapiens
ISPF

SEQ ID NO: 138 - protein, 4 aa, Homo sapiens
HEHA

SEQ ID NO: 139 - protein, 4 aa, Homo sapiens
EVVF

SEQ ID NO: 140 - protein, 4 aa, Homo sapiens
TAND

SEQ ID NO: 141 - protein, 4 aa, Homo sapiens
SGPR

SEQ ID NO: 142 - protein, 4 aa, Homo sapiens
RYTI

SEQ ID NO: 143 - protein, 4 aa, Homo sapiens
AALL

SEQ ID NO: 144 - protein, 4 aa, Homo sapiens
SPYS

SEQ ID NO: 145 - protein, 4 aa, Homo sapiens
YSTT

SEQ ID NO: 146 - protein, 4 aa, Homo sapiens
AVVT

SEQ ID NO: 147 - protein, 4 aa, Homo sapiens
NPKE

SEQ ID NO: 148 - protein, 9 aa, synthetic construct
GGGGCGGGG

SEQ ID NO: 149 - protein, 8 aa, synthetic construct
GGGCGGGG

SEQ ID NO: 150 - protein, 8 aa, synthetic construct
GGCGGGGG

SEQ ID NO: 151 - protein, 8 aa, synthetic construct
GCGGGGGG

SEQ ID NO: 152 - protein, 7 aa, synthetic construct
GGGGCGG

SEQ ID NO: 153 - protein, 7 aa, synthetic construct
GGGCGGG

SEQ ID NO: 154 - protein, 7 aa, synthetic construct
GGCGGGG

SEQ ID NO: 155 - protein, 7 aa, synthetic construct
GCGGGGG

SEQ ID NO: 156 - protein, 6 aa, synthetic construct
GGGCGG

SEQ ID NO: 157 - protein, 6 aa, synthetic construct
GGCGGG

SEQ ID NO: 158 - protein, 6 aa, synthetic construct
GCGGGG

SEQ ID NO: 159 - protein, 4 aa, synthetic construct
GCGG

SEQ ID NO: 160 - protein, 4 aa, synthetic construct
GGCG

SEQ ID NO: 161 - protein, 10 aa, Homo sapiens
TWEPFASGKT